(19) United States
(12) Patent Application Publication
(10) Pub. No.: US 2022/0243201 A1
(43) Pub. Date: Aug. 4, 2022

(54) ENGINEERED ITR SEQUENCES AND METHODS OF USE

(71) Applicant: BIOVERATIV THERAPEUTICS INC., Waltham, MA (US)

(72) Inventors: Ajay Maghodia, Waltham, MA (US); Philip Zakas, Waltham, MA (US)

(21) Appl. No.: 17/576,562

(22) Filed: Jan. 14, 2022

Related U.S. Application Data
(63) Continuation of application No. PCT/US2021/047207, filed on Aug. 23, 2021.
(60) Provisional application No. 63/069,114, filed on Aug. 23, 2020.

(51) Int. Cl. C12N 15/113; A61K 31/713; A61K 31/7105; A61P 7/04; C07K 14/755; C12N 15/86; C12N 7/00

(52) U.S. Cl. CPC C12N 15/113; A61K 31/713; A61K 31/7105; A61P 7/04; C07K 14/755; C12N 15/86; C12N 2830/50; C12N 2750/14143; C12N 2750/14171; C12N 2750/14343; C12N 2750/14371; C12N 2830/42; C12N 7/00

(57) ABSTRACT

The present disclosure provides nucleic acid molecules comprising a first inverted terminal repeat (ITR), a second ITR, and a genetic cassette encoding a target sequence. In some embodiments, the first ITR and/or the second ITR is an ITR of a non-adeno-associated virus (AAV). Also disclosed are methods of using the nucleic acid molecules in gene therapy applications.

### RELATED APPLICATIONS

This application is a continuation of International Patent Application No. PCT/US2021/047207, filed Aug. 23, 2021, which claims priority to U.S. Provisional Patent Application No. 63/069,114, filed Aug. 23, 2020, the entire disclosures of which are hereby incorporated by reference in their entirety.

### REFERENCE TO SEQUENCE LISTING SUBMITTED ELECTRONICALLY

The content of the electronically submitted sequence listing in ASCII text file (Name: 725672_SA9-478PCCON_ST25.txt; Size: 62.7 Kb; Date of Creation: Jan. 14, 2022) is incorporated herein by reference in its entirety.

### BACKGROUND OF THE DISCLOSURE

Gene therapy offers the potential for a lasting means of treating a variety of diseases. In the past, many gene therapy treatments typically relied on the use of viruses. There are numerous viral agents that could be selected for this purpose, each with distinct properties that make them more or less suitable for gene therapy. However, the undesired properties of some viral vectors have resulted in clinical safety concerns and limited their therapeutic use.

Adeno-associated virus (AAV) is a common gene therapy vector, but it is not without its drawbacks. The coding sequences of the AAV genome are flanked by inverted terminal repeats (ITRs) which are required for viral replication and packaging, as well as transgene expression. The T-shaped hairpin structures of AAV ITRs are susceptible to binding by host cell proteins which inhibit transgene expression in AAV vectors. There exists a need to provide efficient and persistent expression of target sequences while avoiding the limitations of existing AAV vector technology.

### SUMMARY OF THE DISCLOSURE

Disclosed herein are nucleic acid molecules and uses thereof comprising a modified first inverted terminal repeat (ITR) and/or a modified second ITR flanking a genetic cassette comprising a heterologous polynucleotide sequence. The modified ITRs disclosed herein provide shorter and/or alternative ITRs to wild type ITRs while retaining a functional property of the wild type ITR. The modified ITRs disclosed herein also provide shorter and/or alternative ITRs to wild type ITRs while retaining a functional property of a protein produced by the genetic cassette.

In one aspect, provided herein is a nucleic acid molecule comprising a first inverted terminal repeat (ITR) and a second ITR flanking a genetic cassette comprising a heterologous polynucleotide sequence, wherein the first ITR comprises a polynucleotide sequence at least about 75% identical to: nucleotides 1-49, 50-58, and 59-125 of SEQ ID NO:1, or nucleotides 1-27 and 50-114 of SEQ ID NO:15; and the second ITR comprises a polynucleotide sequence at least about 75% identical to nucleotides 1-67, 68-76, and 77-125 of SEQ ID NO:2, or nucleotides 1-65 and 88-114 of SEQ ID NO:16, or SEQ ID NOs 25 or 26.

In some embodiments, the first ITR comprises a polynucleotide sequence at least about 80%, at least about 85%, at least about 90%, at least about 95%, at least about 96%, at least about 97%, at least about 98%, at least about 99% identical to: nucleotides 1-49, 50-58, and 59-125 of SEQ ID NO:1, or nucleotides 1-27 and 50-114 of SEQ ID NO:15, or SEQ ID NO: 25. In some embodiments, the first ITR comprises nucleotides 1-49, 50-58, and 59-125 of SEQ ID NO:1, or nucleotides 1-27 and 50-114 of SEQ ID NO:15, or SEQ ID NO: 25.

In some embodiments, the first ITR comprises the polynucleotide sequence set forth in SEQ ID NO:1. In some embodiments, the first ITR comprises the polynucleotide sequence set forth in SEQ ID NO:3. In some embodiments, the first ITR comprises the polynucleotide sequence set forth in SEQ ID NO:5. In some embodiments, the first ITR comprises the polynucleotide sequence set forth in SEQ ID NO:9. In some embodiments, the first ITR comprises the polynucleotide sequence set forth in SEQ ID NO:13. In some embodiments, the first ITR comprises the polynucleotide sequence set forth in SEQ ID NO:15. In some embodiments, the first ITR comprises the polynucleotide sequence set forth in SEQ ID NO:17. In some embodiments, the first ITR comprises the polynucleotide sequence set forth in SEQ ID NO:19. In some embodiments, the first ITR comprises the polynucleotide sequence set forth in SEQ ID NO:21. In some embodiments, the second ITR comprises the nucleotide sequence of SEQ ID NO: 23. In some embodiments, the first ITR comprises the polynucleotide sequence set forth in SEQ ID NO:25.

In some embodiments, the second ITR comprises a polynucleotide sequence at least about 80%, at least about 85%, at least about 90%, at least about 95%, at least about 96%, at least about 97%, at least about 98%, at least about 99% identical to: nucleotides 1-67, 68-76, and 77-125 of SEQ ID NO:2, or nucleotides 1-65 and 88-114 of SEQ ID NO:16, or SEQ ID NO: 26. In some embodiments, the second ITR comprises nucleotides 1-67, 68-76, and 77-125 of SEQ ID NO:2, or nucleotides 1-65 and 88-114 of SEQ ID NO:16, or SEQ ID NO: 26. In some embodiments, the second ITR comprises the nucleotide sequence of SEQ ID NO: 24.

In some embodiments, the second ITR comprises the polynucleotide sequence set forth in SEQ ID NO:2. In some embodiments, the second ITR comprises the polynucleotide sequence set forth in SEQ ID NO:4. In some embodiments, the second ITR comprises the polynucleotide sequence set forth in SEQ ID NO:6. In some embodiments, the second ITR comprises the polynucleotide sequence set forth in SEQ ID NO:10. In some embodiments, the second ITR comprises the polynucleotide sequence set forth in SEQ ID NO:14. In some embodiments, the second ITR comprises the polynucleotide sequence set forth in SEQ ID NO:16. In some embodiments, the second ITR comprises the polynucleotide sequence set forth in SEQ ID NO:18. In some embodiments, the second ITR comprises the polynucleotide sequence set forth in SEQ ID NO:20. In some embodiments, the second ITR comprises the polynucleotide sequence set forth in SEQ ID NO:22. In some embodiments, the second ITR comprises the nucleotide sequence of SEQ ID NO: 24. In some embodiments, the second ITR comprises the polynucleotide sequence set forth in SEQ ID NO:26.

In some embodiments, the first ITR is selected from a polynucleotide sequence set forth in SEQ ID NOs: 1, 3, 5, 9, 13, 15, 17, 19, 21, 23, or 25, and the second ITR is selected from a polynucleotide sequence set forth in SEQ ID NOs: 2, 4, 6, 10, 14, 16, 18, 20, 22, 24, or 26.

In some embodiments, the nucleic acid molecule further comprises a promoter. In some embodiments, the promoter is a tissue-specific promoter. In some embodiments, the promoter drives expression of the heterologous polynucleotide sequence in an organ or tissue, wherein the organ or tissue comprises muscle, central nervous system (CNS), eye, liver, heart, kidney, pancreas, lungs, skin, bladder, urinary tract, spleen, myeloid and lymphoid cell lineages, or any combination thereof. In some embodiments, the promoter drives expression of the heterologous polynucleotide sequence in hepatocytes, epithelial cells, endothelial cells, cardiac muscle cells, skeletal muscle cells, sinusoidal cells, afferent neurons, efferent neurons, interneurons, glial cells, astrocytes, oligodendrocytes, microglia, ependymal cells, lung epithelial cells, Schwann cells, satellite cells, photoreceptor cells, retinal ganglion cells, T cells, B cells, NK cells, macrophages, dendritic cells, or any combination thereof. In some embodiments, the promoter is positioned 5′ to the heterologous polynucleotide sequence. In some embodiments, the promoter is a mouse transthyretin promoter (mTTR), a native human factor VIII promoter, a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, α1-antitrypsin (AAT), muscle creatine kinase (MCK), myosin heavy chain alpha (αMHC), myoglobin (MB), desmin (DES), SPc5-12, 2R5Sc5-12, dMCK, tMCK, or a phosphoglycerate kinase (PGK) promoter. In some embodiments, the promoter comprises the nucleic acid sequence of SEQ ID NO: 31.

In some embodiments, the heterologous polynucleotide sequence further comprises an intronic sequence. In some embodiments, the intronic sequence is positioned 5′ to the heterologous polynucleotide sequence. In some embodiments, the intronic sequence is positioned 3′ to the promoter. In some embodiments, the intronic sequence comprises a synthetic intronic sequence. In some embodiments, the intronic sequence comprises the nucleic acid sequence of SEQ ID NO: 32.

In some embodiments, the genetic cassette further comprises a post-transcriptional regulatory element. In some embodiments, the post-transcriptional regulatory element is positioned 3′ to the heterologous polynucleotide sequence. In some embodiments, the regulatory element comprises a mutated woodchuck hepatitis virus post-transcriptional regulatory element (WPRE), a microRNA binding site, a DNA nuclear targeting sequence, a TLR9 inhibitory sequence, or any combination thereof. In some embodiments, the post-transcriptional regulatory element comprises the nucleic acid sequence of SEQ ID NO: 33.

In some embodiments, the genetic cassette further comprises a 3′UTR poly(A) tail sequence. In some embodiments, the 3′UTR poly(A) tail sequence is selected from the group consisting of bGH poly(A), actin poly(A), hemoglobin poly(A), and any combination thereof.

In some embodiments, the genetic cassette further comprises an enhancer sequence. In some embodiments, the enhancer sequence is positioned between the first ITR and the second ITR. In some embodiments, the enhancer comprises the nucleic acid sequence of SEQ ID NO: 30.

In some embodiments, the nucleic acid molecule comprises from 5′ to 3′: the first ITR, the genetic cassette, and the second ITR, wherein the genetic cassette comprises a tissue-specific promoter sequence, an intronic sequence, the heterologous polynucleotide sequence, a post-transcriptional regulatory element, and a 3′UTR poly(A) tail sequence.

In some embodiments, the genetic cassette comprises from 5′ to 3′: a tissue-specific promoter sequence, an intronic sequence, the heterologous polynucleotide sequence, a post-transcriptional regulatory element, and a 3′UTR poly(A) tail sequence.

In some embodiments, the genetic cassette comprises a single stranded nucleic acid. In some embodiments, the genetic cassette comprises a double stranded nucleic acid.

In some embodiments, the heterologous polynucleotide sequence encodes a therapeutic protein.

In some embodiments, the heterologous polynucleotide sequence encodes a clotting factor, a growth factor, a hormone, a cytokine, an antibody, a fragment thereof, or any combination thereof. In some embodiments, the heterologous polynucleotide sequence encodes a growth factor. In some embodiments, the heterologous polynucleotide sequence encodes a hormone. In some embodiments, the heterologous polynucleotide sequence encodes a cytokine.

In some embodiments, the heterologous polynucleotide sequence encodes an antibody or a fragment thereof.

In some embodiments, the heterologous polynucleotide sequence encodes dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), GALT, OTC, CMD1A, LAMA2, or any combination thereof.

In some embodiments, the heterologous polynucleotide sequence encodes a microRNA (miRNA). In some embodiments, the miRNA down regulates the expression of a target gene comprising SOD1, HTT, RHO, CD38, or any combination thereof.

In some embodiments, the heterologous polynucleotide sequence encodes a clotting factor, wherein the clotting factor comprises factor I (FI), factor II (FII), factor III (FIII), factor IV (FIV), factor V (FV), factor VI (FVI), factor VII (FVII), factor VIII (FVIII), factor IX (FIX), factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), or any combination thereof.

In some embodiments, the heterologous polynucleotide sequence is codon optimized. In some embodiments, the heterologous polynucleotide sequence is codon optimized for expression in a human.

In some embodiments, the nucleic acid molecule is formulated with a delivery agent. In some embodiments, the delivery agent comprises a lipid nanoparticle. In some embodiments, the delivery agent comprises liposomes, non-lipid polymeric molecules, endosomes, or any combination thereof.

In some embodiments, the nucleic acid molecule is formulated for intravenous, transdermal, intradermal, subcutaneous, pulmonary, intraneural, intraocular, intrathecal, or oral administration, or any combination thereof. In some embodiments, the nucleic acid molecule is formulated for intravenous administration. In some embodiments, the nucleic acid molecule is formulated for administration by in situ injection. In some embodiments, the nucleic acid molecule is formulated for administration by inhalation.

In another aspect, provided herein is a vector comprising a nucleic acid molecule described herein.

In another aspect, provided herein is a host cell comprising a nucleic acid molecule described herein, or a vector described herein.

In another aspect, provided herein is a pharmaceutical composition comprising a nucleic acid molecule described herein.

In another aspect, provided herein is a pharmaceutical composition comprising a vector described herein and a pharmaceutically acceptable excipient.

In another aspect, a pharmaceutical composition is provided herein comprising a host cell described herein and a pharmaceutically acceptable excipient.

In another aspect, a kit is provided herein comprising a nucleic acid molecule described herein and instructions for administering the nucleic acid molecule to a subject in need thereof.

In another aspect, provided herein is a baculovirus system for production of a nucleic acid molecule described herein.

In some embodiments, the nucleic acid molecule is produced in insect cells.

In another aspect, provided herein is a nanoparticle delivery system comprising a nucleic acid molecule described herein.

In another aspect, provided herein is a method of expressing a heterologous polynucleotide sequence in a subject in need thereof, comprising administering to the subject a nucleic acid molecule described herein, a vector described herein, or a pharmaceutical composition described herein.

In another aspect, provided herein is a method of treating a disease or disorder in a subject in need thereof, comprising administering to the subject a nucleic acid molecule described herein, a vector described herein, or a pharmaceutical composition described herein.

In some embodiments, the nucleic acid molecule is administered intravenously, transdermally, intradermally, subcutaneously, orally, pulmonarily, intraneurally, intraocularly, intrathecally, or any combination thereof. In some embodiments, the nucleic acid molecule is administered intravenously. In some embodiments, the nucleic acid molecule is administered by in situ injection. In some embodiments, the nucleic acid molecule is administered by inhalation.

In some embodiments, the subject is a mammal. In some embodiments, the subject is a human.

### BRIEF DESCRIPTION OF THE DRAWINGS

FIG. 1A: Graphical depiction of the predicted structures of wild type B19 and truncated B19Δ135 ITRs. Shaded areas indicate the B19 Rep Binding Element (RBE). The Gibbs free energy (ΔG) is provided for each ITR sequence. Note: Graphic does not represent precise location of RBE within ITR.

FIG. 1B: Graphical depiction of the predicted structures of wild type GPV and truncated GPVΔ162 ITRs. Shaded areas indicate the GPV Rep Binding Element (RBE). The Gibbs free energy (ΔG) is provided for each ITR sequence. Note: Graphic does not represent precise location of RBE within ITR.

FIG. 2A: Graphical depiction of the predicted structures of wild type B19, B19Δ151, B19Δ223, and B19_minimal truncated ITRs. Shaded areas indicate the B19 Rep Binding Element (RBE). The Gibbs free energy (ΔG) is provided for each ITR sequence. Note: Graphic does not represent precise location of RBE within ITR.

FIG. 2B: Graphical depiction of the predicted structures of wild type GPV, GPVΔ120, GPVΔ186, and GPV minimal truncated ITRs. Shaded areas indicate the GPV Rep Binding Element (RBE). The Gibbs free energy (ΔG) is provided for each ITR sequence. Note: Graphic does not represent precise location of RBE within ITR.

FIG. 3: Measured FVIII activity in HemA mice receiving 34.7 μg of ssFVIII-DNA (circle) or dsFVIII-DNA (square) of FVIII expression construct flanked by B19_min modified ITRs. Blood samples were taken at 3 and 7 days post-injection.

FIGS. 4-9: Graphical depiction of predicted DNA structure of modified ITR according to SEQ ID NOs. 1-22. Predicted structures were generated using Geneious software (Biomatters Ltd., Auckland, New Zealand)

FIGS. 10A-10D: Schematic representation of modified FVIIIXTEN expression cassettes with modified parvoviral ITRs according to embodiments of the invention. FIG. 10A shows schematic linear map of modified FVIIIXTEN expression cassette flanked by the AAV2 WT ITRs (described in the USPTO Application No. 63/069,073). FIG. 10B shows schematic linear map of modified FVIIIXTEN expression cassette flanked by the HBoV1 WT ITRs. FIG. 10C shows schematic linear map of modified FVIIIXTEN expression cassette flanked by the B19 WT (described in the USPTO Application No. 63/069,073) or B19 Minimal (SEQ ID: 1, SEQ ID:2) ITRs. FIG. 10D shows schematic linear map of modified FVIIIXTEN expression cassette flanked by the GPVΔ186 (SEQ ID: 9, SEQ ID: 10), GPVΔ120 (SEQ ID: 13, SEQ ID: 14), or GPV Minimal (SEQ ID: 15, SEQ: 16) ITRs.

FIG. 11: Schematic representation of approach used for ssDNA generation, where a FVIIIXTEN expression cassette flanked by the parvoviral ITRs was digested with restriction enzymes that recognize the ITR related sequence and produce blunt-end DNA. The digested double-stranded DNA products (FVIII expression cassette and plasmid backbone) were heat denatured (denaturation) at 95° C. followed by cooling (renaturation) at 4° C. to allow the palindromic ITR sequences to form hairpins. The resulting ssFVIIIXTEN (ssDNA) was used for systemic administration via hydrodynamic tail-vein injections in HemA mice.

FIG. 12: Graphical representation of plasma FVIII activity levels measured by the Chromogenix Coatest® SP Factor VIII chromogenic assays. The plasma samples were collected at different intervals from hFVIIIR593C^{+/+}/HemA mice systemically injected via hydrodynamic tail-vein injection with 200, 800, or 1600 μg/kg of single-stranded V2.0 ssFVIIIXTEN (ssDNA) flanked by human Bocavirus (HBoV1), human erythrovirus (B19), Goose Parvovirus (GPV), or their variant or combinations (“hybrids”) of ITRs as indicated. Error bars represent standard deviation.

FIGS. 13A-13B: Representations of the purified ceFVIIIXTEN (ceDNA) obtained from the baculovirus system and their in vivo efficacy studies. FIG. 13A shows an agarose gel image of the purified ceFVIIIXTEN (ceDNA) flanked by the AAV2 WT or HBoV1 WT ITRs obtained from the continuous-elution electrophoresis method. The purity is shown in comparison with the starting material (SM) with arrows indicating DNA bands corresponding to the size of FVIIIXTEN ceDNA vector (ceDNA), baculoviral DNA (vDNA) and Sf9 cell genomic DNA (gDNA). FIG. 13B shows a graphical representation of plasma FVIII activity levels measured by the Chromogenix Coatest® SP Factor VIII chromogenic assays. The plasma samples were collected at different intervals from hFVIIIR593C^{+/+}/HemA mice systemically injected via hydrodynamic tail-vein injection with 80, 40, or 12 μg/kg of ceFVIIIXTEN (ceDNA) flanked by the AAV2 or HBoV1 ITRs as indicated. Error bars represent standard deviation.

### DETAILED DESCRIPTION

Disclosed herein are nucleic acid molecules and uses thereof comprising a modified first inverted terminal repeat (ITR) and/or a modified second ITR flanking a genetic cassette comprising a heterologous polynucleotide sequence. In some embodiments, the first and/or second ITR is derived from parvovirus B19 or goose parvovirus (GPV). The modified ITRs disclosed herein provide shorter and/or alternative ITRs to wild type ITRs while retaining a functional property of the wild type ITR. The modified ITRs disclosed herein also provide shorter and/or alternative ITRs to wild type ITRs while retaining a functional property of a protein produced by the genetic cassette.

Exemplary constructs of the disclosure are illustrated in the accompanying figures and sequence listing. In order to provide a clear understanding of the specification and claims, the following definitions are provided below.

### I. Definitions

It is to be noted that the term “a” or “an” entity refers to one or more of that entity: for example, “a nucleotide sequence” is understood to represent one or more nucleotide sequences. Similarly, “a therapeutic protein” and “a miRNA” is understood to represent one or more therapeutic protein and one or more miRNA, respectively. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Also as used herein, “and/or” refers to and encompasses any and all possible combinations of one or more of the associated listed items, as well as the lack of combinations when interpreted in the alternative con.

“Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

In one embodiment, the initial sequence of the ITR and/or the reverse complement comprise about 2-600 nucleotides, about 2-550 nucleotides, about 2-500 nucleotides, about 2-450 nucleotides, about 2-400 nucleotides, about 2-350 nucleotides, about 2-300 nucleotides, or about 2-250 nucleotides. In some embodiments, the initial sequence and/or the reverse complement comprise about 5-600 nucleotides, about 10-600 nucleotides, about 15-600 nucleotides, about 20-600 nucleotides, about 25-600 nucleotides, about 30-600 nucleotides, about 35-600 nucleotides, about 40-600 nucleotides, about 45-600 nucleotides, about 50-600 nucleotides, about 60-600 nucleotides, about 70-600 nucleotides, about 80-600 nucleotides, about 90-600 nucleotides, about 100-600 nucleotides, about 150-600 nucleotides, about 200-600 nucleotides, about 300-600 nucleotides, about 350-600 nucleotides, about 400-600 nucleotides, about 450-600 nucleotides, about 500-600 nucleotides, or about 550-600 nucleotides. In some embodiments, the initial sequence and/or the reverse complement comprise about 5-550 nucleotides, about 5 to 500 nucleotides, about 5-450 nucleotides, about 5 to 400 nucleotides, about 5-350 nucleotides, about 5 to 300 nucleotides, or about 5-250 nucleotides. In some embodiments, the initial sequence and/or the reverse complement comprise about 10-550 nucleotides, about 15-500 nucleotides, about 20-450 nucleotides, about 25-400 nucleotides, about 30-350 nucleotides, about 35-300 nucleotides, or about 40-250 nucleotides. In certain embodiments, the initial sequence and/or the reverse complement comprise about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, or about 600 nucleotides. In particular embodiments, the initial sequence and/or the reverse complement comprise about 400 nucleotides.

In other embodiments, the initial sequence of the ITR and/or the reverse complement comprise about 2-200 nucleotides, about 5-200 nucleotides, about 10-200 nucleotides, about 20-200 nucleotides, about 30-200 nucleotides, about 40-200 nucleotides, about 50-200 nucleotides, about 60-200 nucleotides, about 70-200 nucleotides, about 80-200 nucleotides, about 90-200 nucleotides, about 100-200 nucleotides, about 125-200 nucleotides, about 150-200 nucleotides, or about 175-200 nucleotides. In other embodiments, the initial sequence and/or the reverse complement comprise about 2-150 nucleotides, about 5-150 nucleotides, about 10-150 nucleotides, about 20-150 nucleotides, about 30-150 nucleotides, about 40-150 nucleotides, about 50-150 nucleotides, about 75-150 nucleotides, about 100-150 nucleotides, or about 125-150 nucleotides. In other embodiments, the initial sequence and/or the reverse complement comprise about 2-100 nucleotides, about 5-100 nucleotides, about 10-100 nucleotides, about 20-100 nucleotides, about 30-100 nucleotides, about 40-100 nucleotides, about 50-100 nucleotides, or about 75-100 nucleotides. In other embodiments, the initial sequence and/or the reverse complement comprise about 2-50 nucleotides, about 10-50 nucleotides, about 20-50 nucleotides, about 30-50 nucleotides, about 40-50 nucleotides, about 3-30 nucleotides, about 4-20 nucleotides, or about 5-10 nucleotides. In another embodiment, the initial sequence and/or the reverse complement consist of two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In other embodiments, an intervening nucleotide between the initial sequence and the reverse complement is (e.g., consists of) 0 nucleotide, 1 nucleotide, two nucleotides, three nucleotides, four nucleotides, five nucleotides, six nucleotides, seven nucleotides, eight nucleotides, nine nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop. In some embodiments, the ITR retains the Rep Binding Element (RBE) of the wild type ITR from which it is derived. Preservation of the RBE may be important for stability of the ITR and manufacturing purposes.

The term “parvovirus” as used herein encompasses the family Parvoviridae, including but not limited to autonomously-replicating parvoviruses and Dependoviruses. The autonomous parvoviruses include, for example, members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus. Exemplary autonomous parvoviruses include, but are not limited to, porcine parvovirus, mice minute virus, canine parvovirus, mink entertitus virus, bovine parvovirus, chicken parvovirus, feline panleukopenia virus, feline parvovirus, goose parvovirus (GPV), H1 parvovirus, muscovy duck parvovirus, snake parvovirus, and B19 virus. Other autonomous parvoviruses are known to those skilled in the art. See, e.g., FIELDS et al. VIROLOGY, volume 2, chapter 69 (4th ed., Lippincott-Raven Publishers).

The term “non-AAV” as used herein encompasses nucleic acids, proteins, and viruses from the family Parvoviridae excluding any adeno-associated viruses (AAV) of the Parvoviridae family. “Non-AAV” includes but is not limited to autonomously-replicating members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers).

The term “derived from,” as used herein, refers to a component that is isolated from or made using a specified molecule or organism, or information (e.g., amino acid or nucleic acid sequence) from the specified molecule or organism. For example, a nucleic acid sequence (e.g., ITR) that is derived from a second nucleic acid sequence (e.g., ITR) can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide.

A “capsid-free” or “capsid-less” vector or nucleic acid molecule refers to a vector construct free from a capsid.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “genetic cassette” or “expression cassette” means a DNA sequence capable of directing expression of a particular polynucleotide sequence in an appropriate host cell, comprising a promoter operably linked to a polynucleotide sequence of interest. A genetic cassette may encompass nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence. In some embodiments, the genetic cassette comprises a polynucleotide which encodes a gene product. In some embodiments, the genetic cassette comprises a polynucleotide which encodes a miRNA. In some embodiments, the genetic cassette comprises a heterologous polynucleotide sequence.

A polynucleotide which encodes a product, e.g., a miRNA or a gene product (e.g., a polypeptide such as a therapeutic protein), can include a promoter and/or other expression (e.g., transcription or translation) control sequences operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control sequences, besides a promoter, for example enhancers, operators, repressors, and transcription termination signals, can also be operably associated with a coding region to direct gene product expression.

“Expression control sequences” refer to regulatory nucleotide sequences, such as promoters, enhancers, terminators, and the like, that provide for the expression of a coding sequence in a host cell. Expression control sequences generally encompass any regulatory nucleotide sequence which facilitates the efficient transcription and translation of the coding nucleic acid to which it is operably linked. Non-limiting examples of expression control sequences include promoters, enhancers, translation leader sequences, introns, polyadenylation recognition sequences, RNA processing sites, effector binding sites, or stem-loop structures. A variety of expression control sequences are known to those skilled in the art. These include, without limitation, expression control sequences which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other expression control sequences include those derived from vertebrate genes such as actin, heat shock protein, bovine growth hormone and rabbit β-globin, as well as other sequences capable of controlling gene expression in eukaryotic cells. Additional suitable expression control sequences include tissue-specific promoters and enhancers as well as lymphokine-inducible promoters (e.g., promoters inducible by interferons or interleukins). Other expression control sequences include intronic sequences, post-transcriptional regulatory elements, and polyadenylation signals. Additional exemplary expression control sequences are discussed elsewhere in the present disclosure.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity. Additional exemplary promoters are discussed elsewhere in the present disclosure.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In some embodiments, the nucleic acid molecule comprises a tissue specific promoter. In certain embodiments, the tissue specific promoter drives expression of the therapeutic protein in the liver, in hepatocytes, and/or endothelial cells. In one particular embodiment, the promoter comprises a TTP promoter. In one particular embodiment, the promoter comprises a mTTR promoter.

The terms “restriction endonuclease” and “restriction enzyme” are used interchangeably and refer to an enzyme that binds and cuts within a specific nucleotide sequence within double stranded DNA.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construct, which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, Wis.), the GCG suite of programs (Wisconsin Package Version 9.0, Genetics Computer Group (GCG), Madison, Wis.), BLASTP, BLASTN, BLASTX (Altschul et al., J. Mol. Biol. 215:403 (1990)), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between a query sequence (e.g. a nucleic acid sequence) and a reference sequence, only nucleotides in the query sequence which match to nucleotides in the reference sequence are used to calculate percent identity. Thus, in determining percent identity between a query sequence or a designated portion thereof (e.g., nucleotides 1-522) and a reference sequence, percent identity will be calculated by dividing the number of matched nucleotides by the total number of nucleotides in the complete query sequence.

As used herein, nucleotides corresponding to nucleotides in a particular sequence of the disclosure are identified by alignment of the sequence of the disclosure to maximize the identity to a reference sequence. The number used to identify an equivalent amino acid in a reference sequence is based on the number used to identify the corresponding amino acid in the sequence of the disclosure.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition.

“Administering,” as used herein, means to give a pharmaceutically acceptable nucleic acid molecule, polypeptide expressed therefrom, or vector comprising the nucleic acid molecule of the disclosure to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The nucleic acid molecules, polypeptides, and vectors can be administered as part of a pharmaceutical composition comprising at least one excipient.

The term “pharmaceutically acceptable” as used herein refer to molecular entities and compositions that are physiologically tolerable and do not typically produce toxicity or an allergic or similar untoward reaction, such as gastric upset, dizziness and the like, when administered to a human. Optionally, as used herein, the term “pharmaceutically acceptable” means approved by a regulatory agency of the federal or a state government or listed in the U.S. Pharmacopeia or other generally recognized pharmacopeia for use in animals, and more particularly in humans.

As used herein, the phrase “subject in need thereof” includes subjects, such as mammalian subjects, that would benefit from administration of a nucleic acid molecule, polypeptide, or vector of the disclosure. In some embodiments, the subject is a human subject. In some embodiments, the subjects are individuals with hemophilia. The subject can be an adult or a minor (e.g., under 12 years old).

As used herein, the term “therapeutic protein” refers to any polypeptide known in the art that can be administered to a subject. In some embodiments, the therapeutic protein comprises a protein selected from a clotting factor, a growth factor, an antibody, a functional fragment thereof, or a combination thereof. As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. “Clotting factor” as used herein includes an activated clotting factor, its zymogen, or an activatable clotting factor. An “activatable clotting factor” is a clotting factor in an inactive form (e.g., in its zymogen form) that is capable of being converted to an active form. The term “clotting factor” includes but is not limited to factor I (FI), factor II (FII), factor III (FIII), factor IV (FIV) factor V (FV), factor FVI (FVI) factor FVII (FVII), factor FVIII (FVIII), factor FIX (FIX), factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI-2), zymogens thereof, activated forms thereof, or any combination thereof.

“Clotting activity,” as used herein, means the ability to participate in a cascade of biochemical reactions that culminates in the formation of a fibrin clot and/or reduces the severity, duration or frequency of hemorrhage or bleeding episode.

A “growth factor,” as used herein, includes any growth factor known in the art including cytokines and hormones.

In some embodiments, the therapeutic protein is encoded by a gene selected from dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), or any combination thereof.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, and two or more combinations thereof.

### II. Nucleic Acid Molecules

Certain aspects of the present disclosure aim to overcome deficiencies of AAV vectors for gene therapy. In particular, certain aspects of the present disclosure are directed to a nucleic acid molecule, comprising a first ITR, a second ITR, and a genetic cassette, e.g., encoding a therapeutic protein and/or a miRNA. In some embodiments, the first ITR and second ITR flank a genetic cassette comprising a heterologous polynucleotide sequence. In some embodiments, the nucleic acid molecule does not comprise a gene encoding a capsid protein, a replication protein, and/or an assembly protein. In some embodiments, the genetic cassette encodes a therapeutic protein. In some embodiments, the therapeutic protein comprises a clotting factor. In some embodiments, the genetic cassette encodes a miRNA. In certain embodiments, the genetic cassette is positioned between the first ITR and the second ITR. In some embodiments, the nucleic acid molecule further comprises one or more noncoding region. In certain embodiments, the one or more non-coding region comprises a promoter sequence, an intron, a post-transcriptional regulatory element, a 3′UTR poly(A) sequence, or any combination thereof.

In one embodiment, the genetic cassette is a single stranded nucleic acid. In another embodiment, the genetic cassette is a double stranded nucleic acid. In another embodiment, the genetic cassette is a closed-end double stranded nucleic acid (ceDNA).

In some embodiments, the nucleic acid molecule comprises: (a) a first ITR that is an ITR derived from a non-AAV family member of Parvoviridae (e.g., a B19 or GPV ITR); (b) a tissue specific promoter sequence, e.g., TTP or TTR promoter; (c) an intron, e.g., a synthetic intron; (d) a nucleotide encoding a miRNA or a therapeutic protein, e.g., a clotting factor; (e) a post-transcriptional regulatory element, e.g., WPRE; (f) a 3′UTR poly(A) tail sequence, e.g., bGHpA; (g) a second ITR that is an ITR derived from a non-AAV family member of Parvoviridae (e.g., a B19 or GPV ITR). In some embodiments, the nucleic acid molecule comprises: (a) a first ITR that is an ITR derived from a non-AAV family member of Parvoviridae (e.g., a B19 or GPV ITR); (b) a tissue specific promoter sequence, e.g., mTTR promoter; (c) an intron, e.g., a synthetic intron; (d) a nucleotide encoding a miRNA or a therapeutic protein, e.g., a clotting factor; (e) a post-transcriptional regulatory element, e.g., WPRE; (f) a 3′UTR poly(A) tail sequence, e.g., bGHpA; (g) a second ITR that is an ITR derived from a non-AAV family member of Parvoviridae (e.g., a B19 or GPV ITR).

In some embodiments, the nucleic acid molecule comprises a first ITR, a second ITR, and a genetic cassette encoding a target sequence, wherein the target sequence encodes a therapeutic protein, wherein the therapeutic protein comprises a factor VIII (FVIII) polypeptide.

In some embodiments, the genetic cassette comprises a nucleotide sequence encoding a codon optimized FVIII driven by a mTTR promoter. In some embodiments, the mTTR promoter comprises the nucleic acid sequence of SEQ ID NO: 31. In some embodiments, the genetic cassette further comprises an A1MB2 enhancer element. In some embodiments, the A1MB2 enhancer element comprises the nucleic acid sequence of SEQ ID NO: 30. In some embodiments, the genetic cassette further comprises a chimeric or synthetic intron. In some embodiments, the chimeric intron consists of chicken beta-actin/rabbit beta-globin intron and has been modified to eliminate five existing ATG sequences to reduce false translation starts. In some embodiments, the intronic sequence is positioned 5′ to the nucleic acid sequence encoding the FVIII polypeptide. In some embodiments, the chimeric intron is positioned 5′ to a promoter sequence, such as the mTTR promoter. In some embodiments, the chimeric intron comprises the nucleic acid sequence of SEQ ID NO: 32. In some embodiments, the genetic cassette further comprises a a Woodchuck Posttranscriptional Regulatory Element (WPRE). In some embodiments, the WPRE comprises the nucleic acid sequence of SEQ ID NO: 33. In some embodiments, the genetic cassette further comprises a Bovine Growth Hormone Polyadenylation (bGHpA) signal. In some embodiments, the bGHpA signal comprises the nucleic acid sequence of SEQ ID NO: 34. In some embodiments, the genetic cassette comprises a nucleotide sequence having at least 70%, 75%, 80%, 85%, 90%, 95%, or 100% sequence identity to SEQ ID NO: 27. In some embodiments, the genetic cassette comprises the nucleotide sequence of SEQ ID NO: 27.

In some embodiments, disclosed herein are isolated nucleic acid molecules encoding a FVIII protein. In some embodiments, disclosed herein are isolated nucleic acid molecules encoding a FVIII protein and comprise a nucleotide sequence at least about 75% identical to SEQ ID NO: 28. In some embodiments, disclosed herein are isolated nucleic acid molecules encoding a FVIII protein and comprise a nucleotide sequence as set forth in SEQ ID NO: 28.

In some embodiments, disclosed herein are isolated nucleic acid molecules encoding a FVIII protein. In some embodiments, disclosed herein are isolated nucleic acid molecules encoding a FVIII protein and comprise a nucleotide sequence at least about 75% identical to SEQ ID NO: 29. In some embodiments, disclosed herein are isolated nucleic acid molecules encoding a FVIII protein and comprise a nucleotide sequence as set forth in SEQ ID NO: 29.

### A. Inverted Terminal Repeats (ITRs)

Certain aspects of the present disclosure are directed to a nucleic acid molecule comprising a first ITR, e.g., a 5′ ITR, and second ITR, e.g., a 3′ ITR. Typically, ITRs are involved in parvovirus (e.g., AAV) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. It is hypothesized that the minimal defining elements indispensable for ITR function are a Rep-binding site and a terminal resolution site plus a variable palindromic sequence allowing for hairpin formation. Palindromic nucleotide regions normally function together in cis as origins of DNA replication and as packaging signals for the virus. Complimentary sequences in the ITRs fold into a hairpin structure during DNA replication. In some embodiments, the ITRs fold into a hairpin T-shaped structure. In other embodiments, the ITRs fold into non-T-shaped hairpin structures, e.g., into a U-shaped hairpin structure. Data suggests that the T-shaped hairpin structures of AAV ITRs may inhibit the expression of a transgene flanked by the ITRs. See, e.g., Zhou et al., Scientific Reports 7:5432 (Jul. 14, 2017). By utilizing an ITR that does not form T-shaped hairpin structures, this form of inhibition may be avoided. Therefore, in certain aspects, a polynucleotide comprising a non-AAV ITR has an improved transgene expression compared to a polynucleotide comprising an AAV ITR that forms a T-shaped hairpin.

In some embodiments, the ITR comprises a naturally occurring ITR, e.g. the ITR comprises all or a portion of an ITR derived from a member of the family Parvoviridae. In some embodiments, the ITR comprises a synthetic sequence. In one embodiment, the first ITR or the second ITR comprises a synthetic sequence. In another embodiment, each of the first ITR and the second ITR comprises a synthetic sequence. In some embodiments, the first ITR or the second ITR comprises a naturally occurring sequence. In another embodiment, each of the first ITR and the second ITR comprises a naturally occurring sequence.

In some embodiments, the ITR comprises or consists of a portion of a naturally occurring ITR, e.g., a truncated ITR. In some embodiments, the ITR comprises or consists of a fragment of a naturally occurring ITR, wherein the fragment comprises at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 125 nucleotides, at least about 150 nucleotides, at least about 175 nucleotides, at least about 200 nucleotides, at least about 225 nucleotides, at least about 250 nucleotides, at least about 275 nucleotides, at least about 300 nucleotides, at least about 325 nucleotides, at least about 350 nucleotides, at least about 375 nucleotides, at least about 400 nucleotides, at least about 425 nucleotides, at least about 450 nucleotides, at least about 475 nucleotides, at least about 500 nucleotides, at least about 525 nucleotides, at least about 550 nucleotides, at least about 575 nucleotides, or at least about 600 nucleotides; wherein the ITR retains a functional property of the naturally occurring ITR. In certain embodiments, the ITR comprises or consists of a fragment of a naturally occurring ITR, wherein the fragment comprises at least about 129 nucleotides; wherein the ITR retains a functional property of the naturally occurring ITR. In certain embodiments, the ITR comprises or consists of a fragment of a naturally occurring ITR, wherein the fragment comprises at least about 102 nucleotides; wherein the ITR retains a functional property of the naturally occurring ITR. In some embodiments, the ITR retains the Rep Binding Element (RBE) of the wild type ITR from which it is derived. In some embodiments, the ITR retains at least one of the RBEs of the wild type ITR from which it is derived. In some embodiments, the ITR retains at least one of the RBEs or a functional portion thereof of the wild type ITR from which it is derived. Preservation of the RBE may be important for stability of the ITR and manufacturing purposes.

In some embodiments, the ITR comprises or consists of a portion of a naturally occurring ITR, wherein the fragment comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the length of the naturally occurring ITR; wherein the fragment retains a functional property of the naturally occurring ITR.

In certain embodiments, the ITR comprises or consists of a sequence that has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to a homologous portion of a naturally occurring ITR, when properly aligned; wherein the ITR retains a functional property of the naturally occurring ITR. In other embodiments, the ITR comprises or consists of a sequence that has a sequence identity of at least 90% to a homologous portion of a naturally occurring ITR, when properly aligned; wherein the ITR retains a functional property of the naturally occurring ITR. In some embodiments, the ITR comprises or consists of a sequence that has a sequence identity of at least 80% to a homologous portion of a naturally occurring ITR, when properly aligned; wherein the ITR retains a functional property of the naturally occurring ITR. In some embodiments, the ITR comprises or consists of a sequence that has a sequence identity of at least 70% to a homologous portion of a naturally occurring ITR, when properly aligned; wherein the ITR retains a functional property of the naturally occurring ITR. In some embodiments, the ITR comprises or consists of a sequence that has a sequence identity of at least 60% to a homologous portion of a naturally occurring ITR, when properly aligned; wherein the ITR retains a functional property of the naturally occurring ITR. In some embodiments, the ITR comprises or consists of a sequence that has a sequence identity of at least 50% to a homologous portion of a naturally occurring ITR, when properly aligned; wherein the ITR retains a functional property of the naturally occurring ITR.

In some embodiments, the ITR comprises an ITR from an AAV genome. In some embodiments, the ITR is an ITR of an AAV genome selected from AAV1, AAV2, AAV3, AAV4, AAV5, AAV6, AAV7, AAV8, AAV9, AAV10, and any combination thereof. In some embodiments, the ITR is an ITR of any AAV genome known to those of skill in the art, including a natural isolate, e.g., a natural human isolate. In a particular embodiment, the ITR is an ITR of the AAV2 genome. In another embodiment, the ITR is a synthetic sequence genetically engineered to include at its 5′ and 3′ ends ITRs derived from one or more of AAV genomes.

In some embodiments, the ITR is not derived from an AAV genome (i.e. the ITR is derived from a virus that is not AAV). In some embodiments, the ITR is an ITR of a non-AAV. In some embodiments, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain embodiments, the ITR is derived from erythrovirus parvovirus B19 (human virus). In another embodiment, the ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY. In some embodiments, the ITR is derived from a porcine parvovirus, e.g., porcine parvovirus U44978. In some embodiments, the ITR is derived from a mice minute virus, e.g., mice minute virus U34256. In some embodiments, the ITR is derived from a canine parvovirus, e.g., canine parvovirus M19296. In some embodiments, the ITR is derived from a mink enteritis virus, e.g., mink enteritis virus D00765. In some embodiments, the ITR is derived from a Dependoparvovirus. In one embodiment, the Dependoparvovirus is a Dependovirus Goose parvovirus (GPV) strain. In a specific embodiment, the GPV strain is attenuated, e.g., GPV strain 82-0321V. In another specific embodiment, the GPV strain is pathogenic, e.g., GPV strain B.

The first ITR and the second ITR of the nucleic acid molecule can be derived from the same genome, e.g., from the genome of the same virus, or from different genomes, e.g., from the genomes of two or more different virus genomes (also known as “hybrid” ITRs). In certain embodiments, the first ITR and the second ITR are derived from the same AAV genome. In a specific embodiment, the two ITRs present in the nucleic acid molecule of the invention are the same, and can in particular be AAV2 ITRs. In other embodiments, the first ITR is derived from an AAV genome and the second ITR is not derived from an AAV genome (e.g., a non-AAV genome). In other embodiments, the first ITR is not derived from an AAV genome (e.g., a non-AAV genome) and the second ITR is derived from an AAV genome. In still other embodiments, both the first ITR and the second ITR are not derived from an AAV genome (e.g., a non-AAV genome). In one particular embodiment, the first ITR and the second ITR are identical.

In some embodiments, the first ITR is derived from a non-AAV genome and the second ITR is derived from a non-AAV genome, wherein the first ITR and the second ITR are derived from the same genome. Non-limiting examples of non-AAV viral genomes are from Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus. In some embodiments, the first ITR is derived from a non-AAV genome and the second ITR is derived from a non-AAV genome, wherein the first ITR and the second ITR are derived from different viral genomes (also known as “hybrid” ITRs). In some embodiments, first ITR is derived from B19 and the second ITR is derived from GPV. In some embodiments, first ITR is derived from GPV and the second ITR is derived from B19.

In some embodiments, the first ITR is derived from an AAV genome, and the second ITR is derived from erythrovirus parvovirus B19 (human virus). In other embodiments, the second ITR is derived from an AAV genome, and the first ITR is derived from erythrovirus parvovirus B19 (human virus).

In some embodiments, the first ITR comprises or consists of all or a portion of an ITR derived from an AAV or non-AAV genome, and the second ITR comprises or consists of all or a portion of an ITR derived from an AAV or non-AAV genome. In some embodiments, a portion of an ITR derived from an AAV or non-AAV genome is a truncated version of a naturally occurring ITR derived from an AAV or non-AAV genome. In some embodiments, a portion of an ITR derived from an AAV or non-AAV genome comprises portions of a naturally occurring ITR derived from an AAV or non-AAV genome. For example, a portion of an ITR derived from an AAV or non-AAV genome comprises portions of a naturally occurring ITR derived from an AAV or non-AAV genome, wherein at least one RBE or a functional portion thereof is preserved.

In certain embodiments, the first ITR and/or the second ITR comprises or consists of all or a portion of an ITR derived from B19. In certain embodiments, the first ITR and/or the second ITR comprises or consists of all or a portion of an ITR derived from B19. In some embodiments, the second ITR is a reverse complement of the first ITR. In some embodiments, the first ITR is a reverse complement of the second ITR. In some embodiments, the first ITR and/or the second ITR derived from B19 is capable of forming a hairpin structure. In certain embodiments, the hairpin structure does not comprise a T-shaped hairpin.

In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs: 1-8, 17, or 18, wherein the first ITR and/or the second ITR retains a functional property of the B19 ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 1-8, 17, or 18, wherein the first ITR and/or the second ITR is capable of forming a hairpin structure. In certain embodiments, the hairpin structure does not comprise a T-shaped hairpin.

In some embodiments, the first ITR and/or the second ITR comprises or consists of a nucleotide sequence selected from SEQ ID NOs: 1-8, 17, or 18. In some embodiments, the first ITR and/or the second ITR comprises or consists of the nucleotide sequence set forth in SEQ ID NO: 1. In some embodiments, the first ITR and/or the second ITR comprises or consists of the nucleotide sequence set forth in SEQ ID NO: 2. In some embodiments, the first ITR and/or the second ITR comprises or consists of the nucleotide sequence set forth in SEQ ID NO: 3. In some embodiments, the first ITR and/or the second ITR comprises or consists of the nucleotide sequence set forth in SEQ ID NO: 4. In some embodiments, the first ITR and/or the second ITR comprises or consists of the nucleotide sequence set forth in SEQ ID NO: 5. In some embodiments, the first ITR and/or the second ITR comprises or consists of the nucleotide sequence set forth in SEQ ID NO: 6. In some embodiments, the first ITR and/or the second ITR comprises or consists of the nucleotide sequence set forth in SEQ ID NO: 7. In some embodiments, the first ITR and/or the second ITR comprises or consists of the nucleotide sequence set forth in SEQ ID NO: 8. In some embodiments, the first ITR and/or the second ITR comprises or consists of the nucleotide sequence set forth in SEQ ID NO: 17. In some embodiments, the first ITR and/or the second ITR comprises or consists of the nucleotide sequence set forth in SEQ ID NO: 18.

In some embodiments, the first ITR is derived from an AAV genome, and the second ITR is derived from goose parvovirus (GPV). In other embodiments, the second ITR is derived from an AAV genome, and the first ITR is derived from goose parvovirus (GPV).

In certain embodiments, the first ITR and/or the second ITR comprises or consists of all or a portion of an ITR derived from GPV. In certain embodiments, the first ITR and/or the second ITR comprises or consists of all or a portion of an ITR derived from GPV. In some embodiments, the second ITR is a reverse complement of the first ITR. In some embodiments, the first ITR is a reverse complement of the second ITR. In some embodiments, the first ITR and/or the second ITR derived from GPV is capable of forming a hairpin structure. In certain embodiments, the hairpin structure does not comprise a T-shaped hairpin.

In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs: 9-16 or 19-22, wherein the first ITR and/or the second ITR retains a functional property of the GPV ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 9-16 or 19-22, wherein the first ITR and/or the second ITR is capable of forming a hairpin structure. In certain embodiments, the hairpin structure does not comprise a T-shaped hairpin.

In some embodiments, the first ITR and/or the second ITR comprises or consists of a nucleotide sequence selected from SEQ ID NOs: 9-16 or 19-22. In some embodiments, the first ITR and/or the second ITR comprises or consists of the nucleotide sequence set forth in SEQ ID NO: 9. In some embodiments, the first ITR and/or the second ITR comprises or consists of the nucleotide sequence set forth in SEQ ID NO: 10. In some embodiments, the first ITR and/or the second ITR comprises or consists of the nucleotide sequence set forth in SEQ ID NO: 11. In some embodiments, the first ITR and/or the second ITR comprises or consists of the nucleotide sequence set forth in SEQ ID NO: 12. In some embodiments, the first ITR and/or the second ITR comprises or consists of the nucleotide sequence set forth in SEQ ID NO: 13. In some embodiments, the first ITR and/or the second ITR comprises or consists of the nucleotide sequence set forth in SEQ ID NO: 14. In some embodiments, the first ITR and/or the second ITR comprises or consists of the nucleotide sequence set forth in SEQ ID NO: 15. In some embodiments, the first ITR and/or the second ITR comprises or consists of the nucleotide sequence set forth in SEQ ID NO: 16. In some embodiments, the first ITR and/or the second ITR comprises or consists of the nucleotide sequence set forth in SEQ ID NO: 19. In some embodiments, the first ITR and/or the second ITR comprises or consists of the nucleotide sequence set forth in SEQ ID NO: 20. In some embodiments, the first ITR and/or the second ITR comprises or consists of the nucleotide sequence set forth in SEQ ID NO: 21. In some embodiments, the first ITR and/or the second ITR comprises or consists of the nucleotide sequence set forth in SEQ ID NO: 22. In some embodiments, the first ITR and/or the second ITR comprises or consists of the nucleotide sequence set forth in SEQ ID NO: 23. In some embodiments, the first ITR and/or the second ITR comprises or consists of the nucleotide sequence set forth in SEQ ID NO: 24. In some embodiments, the first ITR and/or the second ITR comprises or consists of the nucleotide sequence set forth in SEQ ID NO: 25. In some embodiments, the first ITR and/or the second ITR comprises or consists of the nucleotide sequence set forth in SEQ ID NO: 26.

In some embodiments, the first ITR and/or the second ITR comprises a polynucleotide sequence at least about 75%, at least about 80%, at least about 85%, at least about 90%, at least about 95%, at least about 96%, at least about 97%, at least about 98%, at least about 99%, or 100% identical to nucleotides 1-49, 50-58, and 59-125 of SEQ ID NO:1, or nucleotides 1-27 and 50-114 of SEQ ID NO:15, SEQ ID NO: 23, or SEQ ID NO: 25. In some embodiments, the first ITR and/or the second ITR comprises a polynucleotide sequence at least about 75%, at least about 80%, at least about 85%, at least about 90%, at least about 95%, at least about 96%, at least about 97%, at least about 98%, at least about 99%, or 100% identical to nucleotides 1-67, 68-76, and 77-125 of SEQ ID NO: 2, or nucleotides 1-65 and 88-114 of SEQ ID NO:16, SEQ ID NO: 23, or SEQ ID NO: 26.

It will be appreciated to those of skill in the art that any of the first ITR sequences described herein can be matched with any of the second ITR sequences described herein. In some embodiments, the first ITR sequence described herein is a 5′ ITR sequence. In some embodiments, the second ITR sequence described herein is a 3′ ITR sequence. In some embodiments, the second ITR sequence described herein is a 5′ ITR sequence. In some embodiments, the first ITR sequence described herein is a 3′ ITR sequence. Those of skill in the art will be able to determine the suitable orientation of the first and the second ITR described herein with respect to the architecture of a genetic cassette.

In another particular embodiment, the ITR is a synthetic sequence genetically engineered to include at its 5′ and 3′ ends ITRs not derived from an AAV genome. In another particular embodiment, the ITR is a synthetic sequence genetically engineered to include at its 5′ and 3′ ends ITRs derived from one or more of non-AAV genomes. The two ITRs present in the nucleic acid molecule of the invention can be the same or different non-AAV genomes. In particular, the ITRs can be derived from the same non-AAV genome. In a specific embodiment, the two ITRs present in the nucleic acid molecule of the invention are the same, and can in particular be AAV2 ITRs.

In some embodiments, the ITR sequence comprises one or more palindromic sequence. A palindromic sequence of an ITR disclosed herein includes, but is not limited to, native palindromic sequences (i.e., sequences found in nature), synthetic sequences (i.e., sequences not found in nature), such as pseudo palindromic sequences, and combinations or modified forms thereof. A “pseudo palindromic sequence” is a palindromic DNA sequence, including an imperfect palindromic sequence, which shares less than 80% including less than 70%, 60%, 50%, 40%, 30%, 20%, 10%, or 5%, or no, nucleic acid sequence identity to sequences in native AAV or non-AAV palindromic sequence which form a secondary structure. The native palindromic sequences can be obtained or derived from any genome disclosed herein. The synthetic palindromic sequence can be based on any genome disclosed herein.

The palindromic sequence can be continuous or interrupted. In some embodiments, the palindromic sequence is interrupted, wherein the palindromic sequence comprises an insertion of a second sequence. In some embodiments, the second sequence comprises a promoter, an enhancer, an integration site for an integrase (e.g., sites for Cre or Flp recombinase), an open reading frame for a gene product, or a combination thereof.

In some embodiments, the ITRs form hairpin loop structures. In one embodiment, the first ITR forms a hairpin structure. In another embodiment, the second ITR forms a hairpin structure. Still in another embodiment, both the first ITR and the second ITR form hairpin structures. In some embodiments, the first ITR and/or the second ITR does not form a T-shaped hairpin structure. In certain embodiments, the first ITR and/or the second ITR forms a non-T-shaped hairpin structure. In some embodiments, the non-T-shaped hairpin structure comprises a U-shaped hairpin structure.

In some embodiments, an ITR in a nucleic acid molecule described herein may be a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some embodiments, the transcriptionally active element is a constitutive transcriptionally active element. Constitutive transcriptionally active elements provide an ongoing level of gene transcription, and are preferred when it is desired that the transgene be expressed on an ongoing basis. In other embodiments, the transcriptionally active element is an inducible transcriptionally active element. Inducible transcriptionally active elements generally exhibit low activity in the absence of an inducer (or inducing condition), and are up-regulated in the presence of the inducer (or switch to an inducing condition). Inducible transcriptionally active elements may be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements, can be incorporated into an ITR in a variety of ways. In some embodiments, a transcriptionally active element is incorporated 5′ to any portion of an ITR or 3′ to any portion of an ITR. In other embodiments, a transcriptionally active element of a transcriptionally-activated ITR lies between two ITR sequences. If the transcriptionally active element comprises two or more elements which must be spaced apart, those elements may alternate with portions of the ITR. In some embodiments, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these may be adjacent or spaced apart. In addition, protein binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Sp1 site, Inr region, CRE (cAMP regulatory element) site, ATF-1/CRE site, APBβ box, APBα box, CArG box, CCAC box, or any other element involved in transcription as known in the art.

Aspects of the present disclosure provide a method of cloning a nucleic acid molecule described herein, comprising inserting a nucleic acid molecule capable of complex secondary structures into a suitable vector, and introducing the resulting vector into a suitable bacterial host strain. As known in the art, complex secondary structures (e.g., long palindromic regions) of nucleic acids may be unstable and difficult to clone in bacterial host strains. For example, nucleic acid molecules comprising a first ITR and a second ITR (e.g., non-AAV parvoviral ITRs, e.g., B19 or GPV ITRs) of the present disclosure may be difficult to clone using conventional methodologies. Long DNA plindromes inhibit DNA replication and are unstable in the genomes of E. coli, Bacillus, Steptococcus, Streptomyces, S. cerevisiae, mice, and humans. These effects result from the formation of hairpin or cruciform structures by intrastrand base pairing. In E. coli the inhibition of DNA replication can be significantly overcome in SbcC or SbcD mutants. SbcD is the nuclease subunit, and SbcC is the ATPase subunit of the SbcCD complex. The E. coli SbcCD complex is an exonuclease complex responsible for preventing the replication of long palindromes. The SbcCD complex is a nuclear with ATP-dependent double-stranded DNA exonuclease activity and ATP-independent single-stranded DNA endonuclease activity. SbcCD may recognize DNA plaindromes and collapse replication forks by attacking hairpin structures that arise.

In certain embodiments, a suitable bacterial host strain is incapable of resolving cruciform DNA structures. In certain embodiments, a suitable bacterial host strain comprises a disruption in the SbcCD complex. In some embodiments, the disruption in the SbcCD complex comprises a genetic disruption in the SbcC gene and/or SbcD gene. In certain embodiments, the disruption in the SbcCD complex comprises a genetic disruption in the SbcC gene. Various bacterial host strains that comprise a genetic disruption in the SbcC gene are known in the art. For example, without limitation, the bacterial host strain PMC103 comprises the genotype sbcC, recD, mcrA, ΔmcrBCF; the bacterial host strain PMC107 comprises the genotype recBC, recJ, sbcBC, mcrA, ΔmcrBCF; and the bacterial host strain SURE comprises the genotype recB, recJ, sbcC, mcrA, ΔmcrBCF, umuC, uvrC. Accordingly, in some embodiments a method of cloning a nucleic acid molecule described herein comprises inserting a nucleic acid molecule capable of complex secondary structures into a suitable vector, and introducing the resulting vector into host strain PMC103, PMC107, or SURE. In certain embodiments, the method of cloning a nucleic acid molecule described herein comprises inserting a nucleic acid molecule capable of complex secondary structures into a suitable vector, and introducing the resulting vector into host strain PMC103.

Suitable vectors are known in the art and described elsewhere herein. In certain embodiments, a suitable vector for use in a cloning methodology of the present disclosure is a low copy vector. In certain embodiments, a suitable vector for use in a cloning methodology of the present disclosure is pBR322.

Accordingly, the present disclosure provides a method of cloning a nucleic acid molecule, comprising inserting a nucleic acid molecule capable of complex secondary structures into a suitable vector, and introducing the resulting vector into a bacterial host strain comprising a disruption in the SbcCD complex, wherein the nucleic acid molecule comprises a first inverted terminal repeat (ITR) and a second ITR, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs. 1 to 22 or a functional derivative thereof.

### B. Therapeutic Proteins

Certain aspects of the present disclosure are directed to a nucleic acid molecule comprising a first ITR, a second ITR, and a genetic cassette encoding a target sequence, wherein the target sequence encodes a therapeutic protein. In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

Certain embodiments of the present disclosure are directed to a nucleic acid molecule comprising a first ITR, a second ITR, and a genetic cassette encoding a therapeutic protein, wherein the therapeutic protein comprises a clotting factor. In some embodiments, the clotting factor is selected from the group consisting of FI, FII, FIII, FIV, FV, FVI, FVII, FVIII, FIX, FX, FXI, FXII, FXIII, VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In one embodiment, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

In some embodiments, the nucleic acid molecule comprises a first ITR, a second ITR, and a genetic cassette encoding a target sequence, wherein the target sequence encodes a therapeutic protein, wherein the therapeutic protein comprises a factor VIII polypeptide. “Factor VIII,” abbreviated throughout the instant application as “FVIII,” as used herein, means functional FVIII polypeptide in its normal role in coagulation, unless otherwise specified. Thus, the term FVIII includes variant polypeptides that are functional. “A FVIII protein” is used interchangeably with FVIII polypeptide (or protein) or FVIII. Examples of the FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa.

The FVIII portion in the therapeutic protein used herein has FVIII activity. FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is used in conjunction with prothrombin time (PT), which measures the extrinsic pathway.

ROTEM analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The chromogenic assay mechanism is based on the principles of the blood coagulation cascade, where activated FVIII accelerates the conversion of Factor X into Factor Xa in the presence of activated Factor IX, phospholipids and calcium ions. The Factor Xa activity is assessed by hydrolysis of a p-nitroanilide (pNA) substrate specific to Factor Xa. The initial rate of release of p-nitroaniline measured at 405 nM is directly proportional to the Factor Xa activity and thus to the FVIII activity in the sample. The chromogenic assay is recommended by the FVIII and Factor IX Subcommittee of the Scientific and Standardization Committee (SSC) of the International Society on Thrombosis and Hemostatsis (ISTH). Since 1994, the chromogenic assay has also been the reference method of the European Pharmacopoeia for the assignment of FVIII concentrate potency.

In some embodiments, the genetic cassette comprises a nucleotide sequence encoding a FVIII polypeptide, wherein the nucleotide sequence is codon optimized. In some embodiments, the genetic cassette comprises a nucleotide sequence encoding a codon optimized FVIII driven by a mTTR promoter and synthetic intron. In some embodiments, the genetic cassette comprises a nucleotide sequence which is disclosed in International Application No. PCT/US2017/015879, which is incorporated by reference in its entirety. In some embodiments, the genetic cassette is a “hFVIIIco6XTEN” genetic cassette as described in PCT/US2017/015879.

In some embodiments, the nucleic acid molecule comprises a first ITR, a second ITR, and a genetic cassette encoding a target sequence, wherein the target sequence encodes a therapeutic protein, and wherein the therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7.

In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin B1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is foetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP)). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

### C. Expression Control Sequences

In some embodiments, the nucleic acid molecule of the disclosure further comprises at least one expression control sequence. An expression control sequence, as used herein, is any regulatory nucleotide sequence, such as a promoter sequence or promoter-enhancer combination, which facilitates the efficient transcription and translation of the coding nucleic acid to which it is operably linked. For example, the nucleic acid molecule of the disclosure can be operably linked to at least one transcription control sequence. The gene expression control sequence can, for example, be a mammalian or viral promoter, such as a constitutive or inducible promoter.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. In certain embodiments, the promoter or other expression control sequence selective enhances expression of the transgene in hepatocytes, sinusoidal cells, and/or endothelial cells. In one particular embodiment, the promoter or other expression control sequence selective enhances expression of the transgene in endothelial cells. In certain embodiments, the promoter or other expression control sequence selective enhances expression of the transgene in muscle cells, the central nervous system, the eye, the liver, the heart, or any combination thereof. Examples of liver specific promoters include, but are not limited to, a mouse thyretin promoter (mTTR), an endogenous human factor VIII promoter, human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The FVIII promoter is described in Figueiredo and Brownlee, 1995, J. Biol. Chem. 270:11828-11838. In some embodiments, the promoter is selected from a liver specific promoter (e.g., al-antitrypsin (AAT)), a muscle specific promoter (e.g., muscle creatine kinase (MCK), myosin heavy chain alpha (aMHC), myoglobin (MB), and desmin (DES)), a synthetic promoter (e.g., SPc5-12, 2R5Sc5-12, dMCK, and tMCK), and any combination thereof.

In one embodiment, the promoter is selected from the group consisting of a mouse transthyretin promoter (mTTR), a native human factor VIII promoter, human alpha-1-antitrypsin promoter (hAAT), human albumin minimal promoter, mouse albumin promoter, TTPp, a CASI promoter, a CAG promoter, a cytomegalovirus (CMV) promoter, al-antitrypsin (AAT), muscle creatine kinase (MCK), myosin heavy chain alpha (aMHC), myoglobin (MB), desmin (DES), SPc5-12, 2R5Sc5-12, dMCK, and tMCK, a phosphoglycerate kinase (PGK) promoter and any combination thereof. In some embodiments, the promoter is a TTP promoter. In some embodiments, the promoter is a mouse transthyretin promoter (mTTR) promoter.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In one embodiment, the nucleic acid molecules of the present disclosure further comprises an intronic sequence. In some embodiments, the intronic sequence is positioned 5′ to the nucleic acid sequence encoding the FVIII polypeptide. In some embodiments, the intronic sequence is a naturally occurring intronic sequence. In some embodiments, the intronic sequence is a synthetic sequence. In some embodiments, the intronic sequence is derived from a naturally occurring intronic sequence. In certain embodiments, the intronic sequence comprises the SV40 small T intron.

In some embodiments, the nucleic acid molecule further comprises a post-transcriptional regulatory element. In certain embodiments, the regulatory element comprises a woodchuck hepatitis virus regulatory element (WPRE). In some embodiments, the WPRE is mutated.

In some embodiments, the nucleic acid molecule comprises a microRNA (miRNA) binding site. In one embodiment, the miRNA binding site is a miRNA binding site for miR-142-3p. In other embodiments, the miRNA binding site is selected from a miRNA binding site disclosed by Rennie et al., RNA Biol. 13(6):554-560 (2016), and STarMirDB, available at http://sfold.wadsworth.org/starmirDB.php, which are incorporated by reference herein in their entirety.

In some embodiments, the nucleic acid molecule comprises one or more DNA nuclear targeting sequences (DTSs). A DTS promotes translocation of DNA molecules containing such sequences into the nucleus. In certain embodiments, the DTS comprises an SV40 enhancer sequence. In certain embodiments, the DTS comprises a c-Myc enhancer sequence. In some embodiments, DTSs are between the first ITR and the second ITR. In some embodiments, the DTS is 3′ to the first ITR and 5′ to the therapeutic protein. In other embodiments, the DTS is 3′ to the therapeutic protein and 5′ to the second ITR.

In some embodiments, the nucleic acid molecule further comprises a 3′UTR poly(A) tail sequence. In one embodiment, the 3′UTR poly(A) tail sequence comprises bGH poly(A). In one embodiment, the 3′UTR poly(A) tail comprises an actin poly(A) site. In one embodiment, the 3′UTR poly(A) tail comprises a hemoglobin poly(A) site.

In some embodiments, the transgene expression is targeted to the liver. In certain embodiments, the transgene expression is targeted to hepatocytes. In other embodiment, the transgene expression is targeted to endothelial cells. In one particular embodiment, the transgene expression is targeted to any tissue that naturally expressed endogenous FVIII.

In some embodiments, the transgene expression is targeted to the central nervous system. In certain embodiments, the transgene expression is targeted to neurons. In some embodiments, the transgene expression is targeted to afferent neurons. In some embodiments, the transgene expression is targeted to efferent neurons. In some embodiments, the transgene expression is targeted to interneurons. In some embodiments, the transgene expression is targeted to glial cells. In some embodiments, the transgene expression is targeted to astrocytes. In some embodiments, the transgene expression is targeted to oligodendrocytes. In some embodiments, the transgene expression is targeted to microglia. In some embodiments, the transgene expression is targeted to ependymal cells. In some embodiments, the transgene expression is targeted to Schwann cells. In some embodiments, the transgene expression is targeted to satellite cells.

In some embodiments, the transgene expression is targeted to muscle tissue. In some embodiments, the transgene expression is targeted to smooth muscle. In some embodiments, the transgene expression is targeted to cardiac muscle. In some embodiments, the transgene expression is targeted to skeletal muscle.

In some embodiments, the transgene expression is targeted to the eye. In some embodiments, the transgene expression is targeted to a photoreceptor cell. In some embodiments, the transgene expression is targeted to retinal ganglion cell.

### III. Host Cells

The disclosure also provides a host cell comprising a nucleic acid molecule or vector of the disclosure. As used herein, the term “transformation” shall be used in a broad sense to refer to the introduction of DNA into a recipient host cell that changes the genotype and consequently results in a change in the recipient cell.

“Host cells” refers to cells that have been transformed with vectors constructed using recombinant DNA techniques and encoding at least one heterologous gene. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/0 (mouse myeloma), P3x63-Ag8.653 (mouse myeloma), BFA-1c1BPT (bovine endothelial cells), RAJI (human lymphocyte), PER.C6®, NS0, CAP, BHK21, and HEK 293 (human kidney). In one particular embodiment, the host cell is selected from the group consisting of: a CHO cell, a HEK293 cell, a BHK21 cell, a PER.C6® cell, a NS0 cell, a CAP cell and any combination thereof. In some embodiments, the host cells of the present disclosure are of insect origin. In one particular embodiment, the host cells are SF9 cells. Host cell lines are typically available from commercial services, the American Tissue Culture Collection, or from published literature.

Introduction of the nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

Host cells comprising the isolated nucleic acid molecules or vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, Calif.). In another embodiment, the medium is CD17 (Invitrogen, Carlsbad, Calif.). Selection of a medium appropriate for the particular cell line used is within the level of those ordinary skilled in the art.

### IV. Pharmaceutical Compositions

Compositions containing a nucleic acid molecule, a polypeptide encoded by the nucleic acid molecule, a vector, or a host cell of the present disclosure can contain a suitable pharmaceutically acceptable carrier. For example, they can contain excipients and/or auxiliaries that facilitate processing of the active compounds into preparations designed for delivery to the site of action.

In one embodiment, the present disclosure is directed to a pharmaceutical composition comprising (a) a nucleic acid molecule, a vector, a polypeptide, or a host cell disclosed herein; and (b) a pharmaceutically acceptable excipient.

In some embodiments, the pharmaceutical composition further comprises a delivery agent. In certain embodiments, the delivery agent comprises a lipid nanoparticle (LNP). In other embodiments, the pharmaceutical composition further comprises liposomes, other polymeric molecules, and exosomes.

As used herein a “lipid nanoparticle” refers to a nanoparticle that comprises a plurality of lipid molecules physically associated with each other by intermolecular forces. The lipid nanoparticles may be, e.g., microspheres (including unilamellar and multilamellar vesicles, e.g. liposomes), a dispersed phase in an emulsion, micelles or an internal phase in a suspension.

In some embodiments, the present disclosure provides an encapsulated nucleic acid molecule composition which may include a lipid nanoparticle host encapsulating a nucleic acid molecule of the invention. The lipid nanoparticle may comprise one or more lipids (e.g., cationic lipids, non-cationic lipids, and PEG-modified lipids). In certain embodiments, lipid nanoparticles of the present disclosure are formulated to deliver one or more nucleic acid molecules of the invention to one or more target cells. Examples of suitable lipids include, without limitation, phosphatidyl compounds (e.g., phosphatidylethanolamine, sphingolipids, phosphatidylcholine, phosphatidylserine, phosphatidylglycerol, gangliosides, and cerebrosides). A “cationic lipid” refers to any lipid species that carry a net positive charge at a certain pH (e.g., physiological pH).

In certain embodiments, the lipid nanoparticles of the present disclosure have a certain N/P ratio. As used herein “N/P ratio” or “NP ratio” refers to the ratio of positively-chargeable polymer amine groups to negatively-charged nucleic acid phosphate groups. The N/P character of a lipid nanoparticle/nucleic acid molecule complex can influence properties such as net surface charge, stability, and size. The NP ratio of the lipid nanoparticles as described herein may be about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, and any ratio in between. For example, the NP ratio of the lipid nanoparticles as described herein may be about 18, about 36, or about 72.

Accordingly, in certain embodiments, a pharmaceutical composition comprises a nucleic acid molecule of the present disclosure encapsulated in a lipid nanoparticle, and a pharmaceutically acceptable excipient.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the nucleic acid molecule of the disclosure is formulated with a clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In some embodiments, the composition is administered by a route selected from the group consisting of topical administration, intraocular administration, parenteral administration, intrathecal administration, subdural administration and oral administration. The parenteral administration can be intravenous or subcutaneous administration.

### V. Methods of Treatment

In some aspects, the present disclosure is directed to methods of treating a disease or condition in a subject in need thereof, comprising administering a nucleic acid molecule, a vector, a polypeptide, or a pharmaceutical composition disclosed herein.

In some embodiments, the disclosure is directed to methods of treating a bleeding disorder. In some embodiments, the disclosure is directed to methods of treating hemophilia A.

The isolated nucleic acid molecule, vector, or polypeptide can be administered intravenously, subcutaneously, intramuscularly, or via any mucosal surface, e.g., orally, sublingually, buccally, sublingually, nasally, rectally, vaginally or via pulmonary route. The clotting factor protein can be implanted within or linked to a biopolymer solid support that allows for the slow release of the chimeric protein to the desired site.

For oral administration, the pharmaceutical composition can take the form of tablets or capsules prepared by conventional means. The composition can also be prepared as a liquid for example a syrup or a suspension. The liquid can include suspending agents (e.g. sorbitol syrup, cellulose derivatives or hydrogenated edible fats), emulsifying agents (lecithin or acacia), non-aqueous vehicles (e.g. almond oil, oily esters, ethyl alcohol, or fractionated vegetable oils), and preservatives (e.g. methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration, the composition can take the form of tablets, lozenges or fast dissolving films according to conventional protocols.

For administration by inhalation, the polypeptide having clotting factor activity for use according to the present disclosure are conveniently delivered in the form of an aerosol spray from a pressurized pack or nebulizer (e.g., in PBS), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In one embodiment, the route of administration of the isolated nucleic acid molecule, vector, or polypeptide is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the isolated nucleic acid molecule, vector, or polypeptide can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringers dextrose, dextrose and sodium chloride, lactated Ringers, or fixed oils. Intravenous vehicles include fluid and nutrient replenishers, electrolyte replenishers, such as those based on Ringers dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The nucleic acid molecule, vector, or polypeptides of the disclosure can optionally be administered in combination with other agents that are effective in treating the disorder or condition in need of treatment (e.g., prophylactic or therapeutic).

As used herein, the administration of isolated nucleic acid molecules, vectors, or polypeptides of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the isolated nucleic acid molecule, vector, or polypeptide of the instant disclosure can be used in conjunction or combination with an agent or agents (e.g., to provide a combined therapeutic regimen). Exemplary agents with which a polypeptide or polynucleotide of the disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polynucleotides or polypeptides of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9th ed. 1996). In another embodiment, an amount of such an agent consistent with the standard of care is administered.

In one embodiment, also disclosed herein is a kit, comprising the nucleic acid molecule disclosed herein and instructions for administering the nucleic acid molecule to a subject in need thereof. In another embodiment, disclosed herein is a baculovirus system for production of the nucleic acid molecule provided herein. The nucleic acid molecule is produced in insect cells. In another embodiment, a nanoparticle delivery system for expression constructs is provided. The expression construct comprises the nucleic acid molecule disclosed herein.

### VI. Gene Therapy

Certain aspects of the present disclosure provide a method of expressing a genetic expression construct in a subject, comprising administering the isolated nucleic acid molecule of the disclosure to a subject in need thereof. In some aspects, the disclosure provides a method of increasing expression of a polypeptide in a subject comprising administering the isolated nucleic acid molecule of the disclosure to a subject in need thereof.

Somatic gene therapy has been explored as a possible treatment for a variety of conditions, including, but not limited to, hemophilia A. Gene therapy is a particularly appealing treatment for hemophilia because of its potential to cure the disease through continuous endogenous production of a clotting factor, e.g., FVIII, following a single administration of vector. Hemophilia A is well suited for a gene replacement approach because its clinical manifestations are entirely attributable to the lack of a single gene product (e.g., FVIII) that circulates in minute amounts (200 ng/ml) in the plasma.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

### EXAMPLES

Having provided the foregoing disclosure, a further understanding can be obtained by reference to the examples provided herein. These examples are for purposes of illustration only and are not intended to be limiting.

### Example 1: Design and Construction of Modified GPV and B19 ITRs

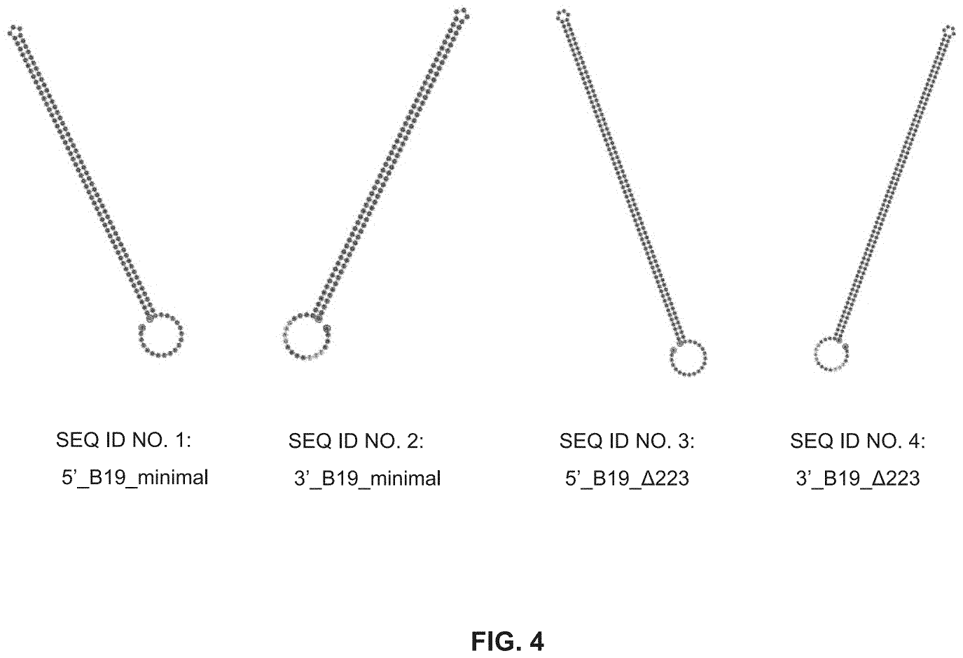

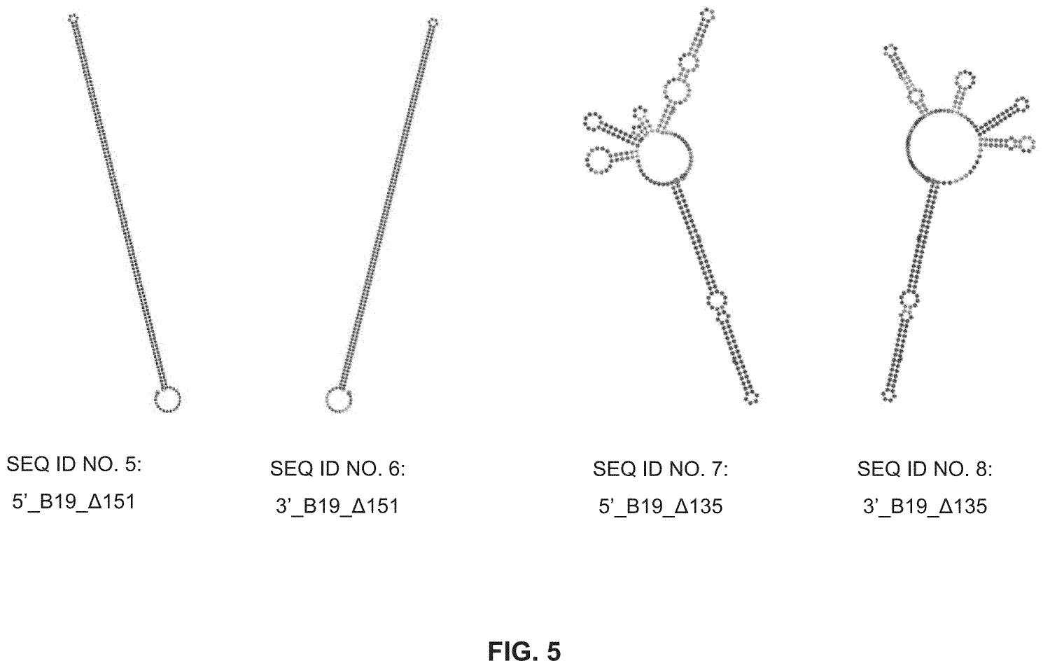

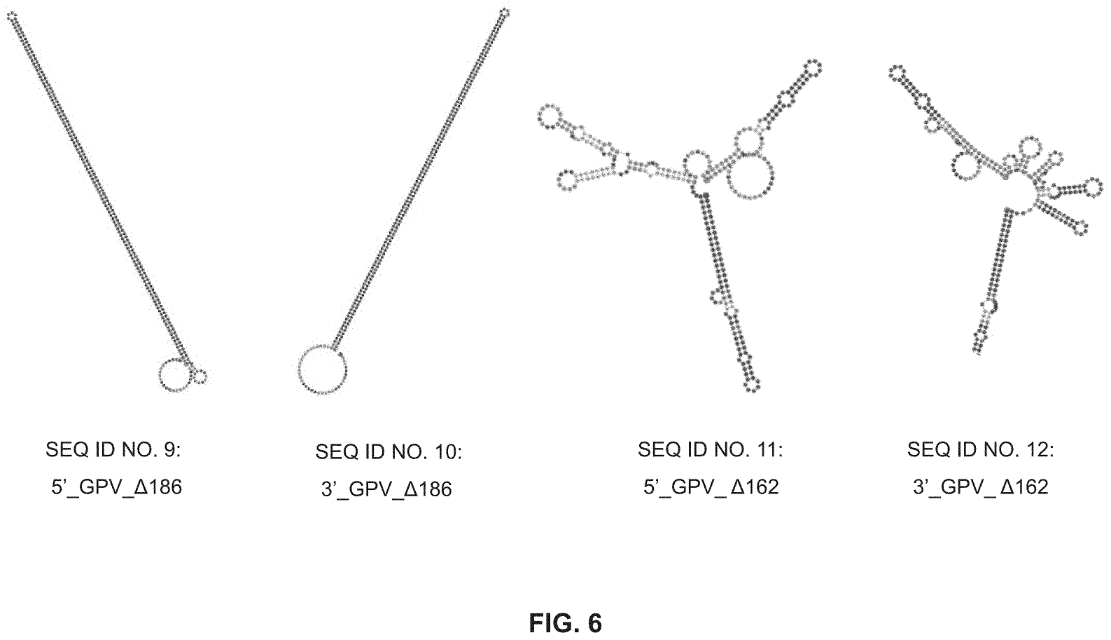

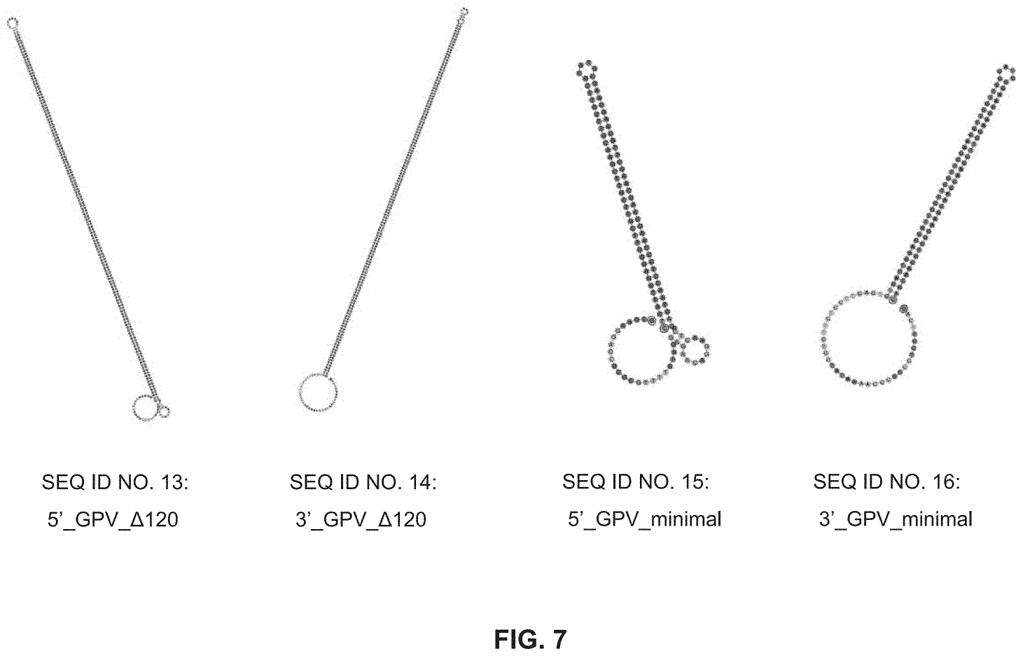

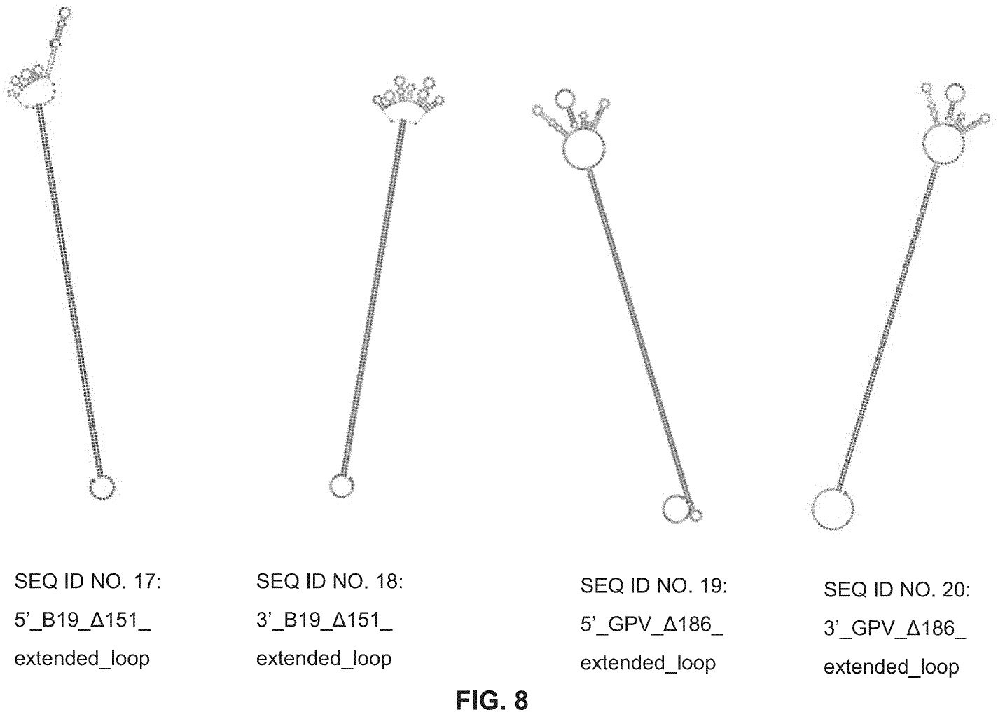

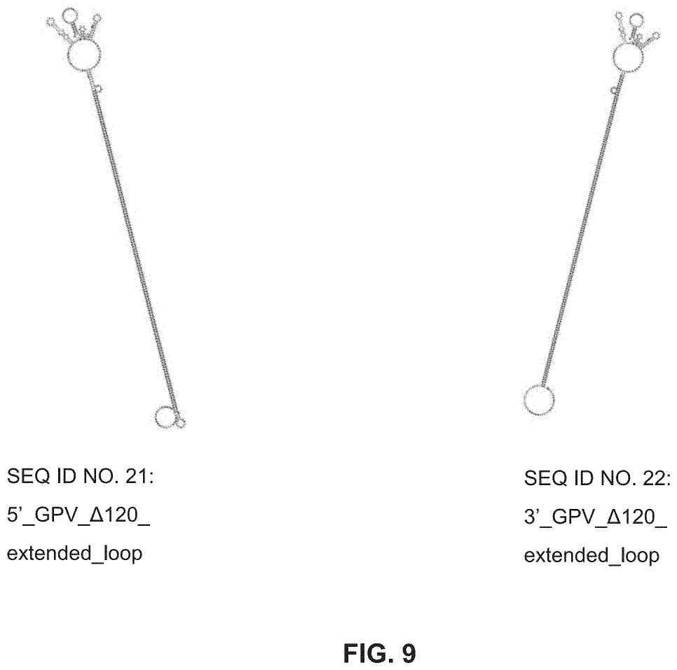

The ITR sequences of AAV2 (Gene Bank accession number NC_001401.2), dependovirus GPV (Gene Bank accession number U25749.1), and erythrovirus B19 (Gene Bank accession number KY940273.1) were analyzed. Based on this analysis, modified derivatives of wild type GPV and B19 ITRs were designed to investigate which nucleic acid sequences of the GPV and B19 ITRs are required for persistent transduction of eukaryotic cells with genetic constructs bearing the modified ITRs. The nucleic acid sequences of exemplary modified ITRs are provided in Table 2. Predicted DNA structures of each of the modified ITRs are shown in FIGS. 4-9.

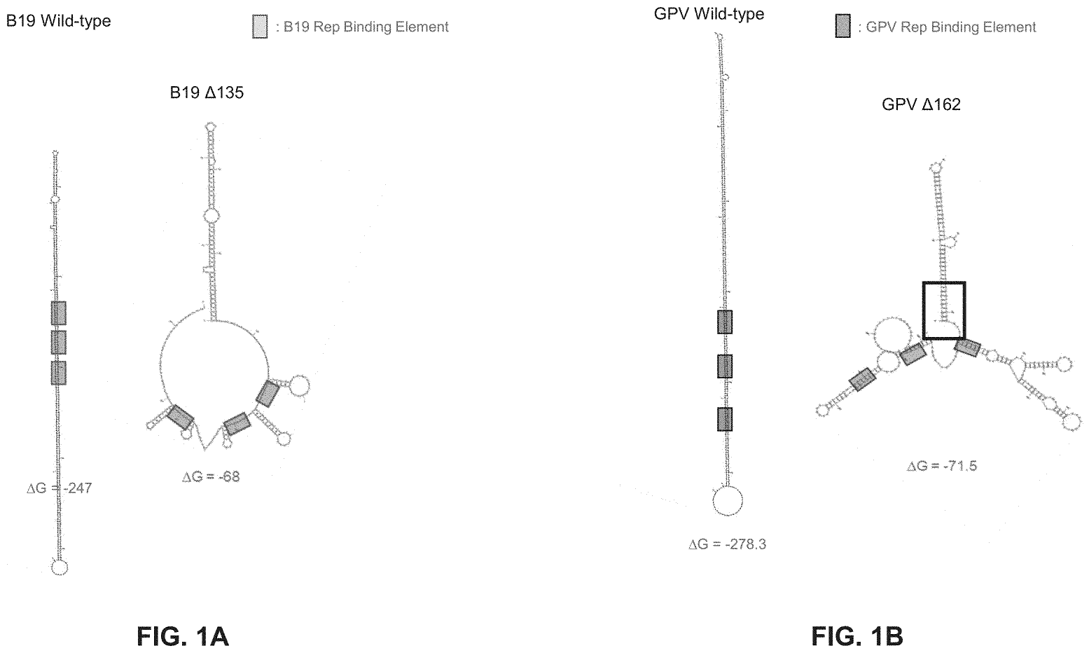

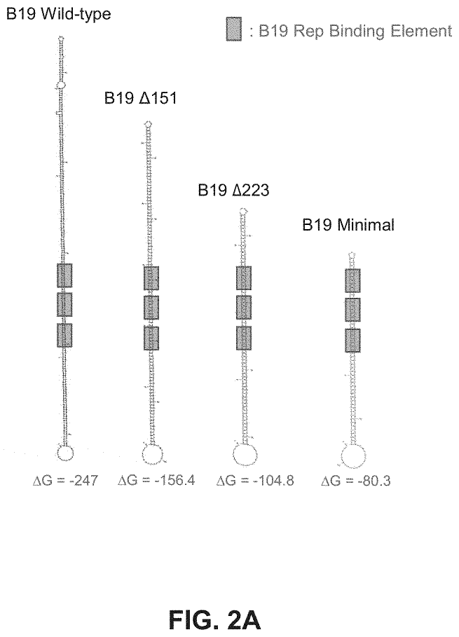

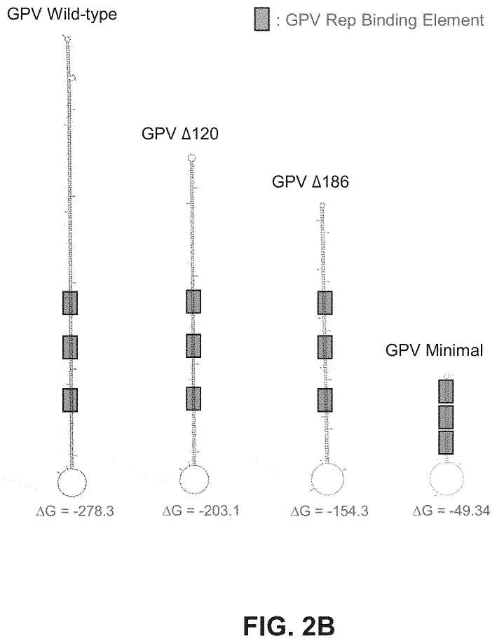

The modified ITR sequences of SEQ ID NOs. 1 to 8 are truncated derivatives of wild type B19 ITR. Graphical depictions of the predicted structures of truncated B19 ITR derivatives are shown in FIGS. 1A and 2A. The modified ITR sequences of SEQ ID NOs. 9 to 16 are truncated derivatives of wild type GPV ITR. Graphical depictions of the predicted structures of truncated GBV ITR derivatives are shown in FIGS. 1B and 2B. These truncated ITRs were designed to maintain the hairpin structure of the wildtype ITR and to preserve one or more of the Rep Binding Elements (RBEs) within the sequence. The bases removed in these sequences are nucleotides located between the RBEs and the dyad along with their corresponding binding partner on the other side of the hairpin. The modified ITR sequences of SEQ ID NOs. 1 to 16 have a range of hairpin length and thermodynamic stability which are predicted to contribute to in vivo stability and efficacy as well as improved manufacturability and product stability.

The modified ITR sequences of SEQ ID NOs. 1 and 2 (B19_min) contain further truncations of the B19 ITR sequence. A graphical depiction of the predicted structure of the derivative B19_min ITR is shown in FIG. 2A.

The modified ITR sequences of SEQ ID NOs. 15 and 16 (GPV_min) contain further truncations of the GPV ITR sequence which removes nucleotides between, but not contributing to, the RBEs. A graphical depiction of the predicted structure of the derivative GPV_min ITR is shown in FIG. 2B.

The modified ITR sequences of SEQ ID NOs. 17 to 22 contain truncated ITR sequences with extra nucleotides inserted at the dyad in order to improve de novo synthesis and subsequent cloning efforts.

It was hypothesized that, due to the retained presence of the RBE within the ITR, genetic expression constructs bearing these modified derivative ITRs will remain functional and effectively transduce eukaryotic cells.

### Example 2. Generation of FVIII Expression Constructs with Modified ITRs

Genetic constructs were created using a plasmid encoding a FVIII genetic cassette, which comprises a codon optimized FVIII with mTTR promoter and synthetic intron.

New constructs will be generated which comprise the codon optimized FVIII genetic cassette flanked by a modified ITR set forth in SEQ ID NOs. 1 to 22 at either or both of the 5′ or 3′ ITR locations. Exemplary constructs and modified ITR pairs are shown in Table 1.

These new constructs were grown and maintained in E. coli strain PMC103 containing deleted sbcC gene, which encodes an exonuclease that recognizes and eliminates cruciform DNA structures. The PMC103 E. coli was able to support the growth of the codon optimized FVIII constructs with modified ITRs after optimizing the temperature and the growth conditions of bacterial cultures. All new constructs were selected on ampicillin resistant plates and screened by restriction enzyme mapping to confirm the correct genetic structures. Modified ITRs were also Sanger sequenced following restriction digest and gel extraction.

TABLE 1
Exemplary FVIII constructs and ITR pairs.

| | | 5′ ITR | 3′ ITR |
|---|---|---|---|
| FVIII | | (SEQ ID | (SEQ ID |
| Construct | Description of ITRs | NO.) | NO.) |
| Construct-1 | B19_min | 1 | 2 |
| Construct-2 | B19_Δ223 | 3 | 4 |
| Construct-3 | B19_Δ151 | 5 | 6 |
| Construct-4 | B19_Δ135 | 7 | 8 |
| Construct-4 | GPV_Δ186 | 9 | 10 |
| Construct-5 | GPV_Δ162 | 11 | 12 |
| Construct-6 | GPV_Δ120 | 13 | 14 |
| Construct-7 | GPV_min | 15 | 16 |
| Construct-8 | B19_Δ151_extended_loop | 17 | 18 |
| Construct-9 | GPV_Δ186_extended_loop | 19 | 20 |
| Construct-10 | GPV_Δ120_extended_loop | 21 | 22 |

### Example 3. Preparation of Single-Stranded DNA Fragments Containing FVIII Expression Cassettes Flanked with Modified ITRs

It was hypothesized that formation of hairpin structures within the ITR regions flanking the codon optimized FVIII expression cassette would drive persistent transduction of target cells. For each plasmid construct, single-stranded (ss) DNA fragments with formed hairpin ITR structures were generated by denaturing the double-stranded DNA fragment products (FVIII expression cassette and plasmid backbone) of PvuII or LguI digestion at 95° C. and then cooling down at 4° C. to allow the palindromic ITR sequences to fold.

### Example 4. In Vivo Evaluation of FVIII Expression from ssDNA and dsDNA Constructs of B19 Minimal ITRs

To validate the ability of expression constructs bearing modified ITR regions to mediate persistent transgene expression in vivo, 34.7 ug DNA of the pFVIII.B19_min construct, which contains a codon optimized FVIII transgene flanked by B19 minimal ITRs (5′ ITR: SEQ ID NO. 1 and 3′ ITR: SEQ ID NO: 2), was delivered systemically via hydrodynamic injection (HDI) to 8-12 week old male hemophilia (HemA) mice. DNA was administered in either double-stranded (dsDNA) format without pre-formed hairpins or as single-stranded DNA (ssDNA) in which the ITR hairpins are pre-formed, as prepared in Example 3. Plasma samples were collected from experimental animals at 3 days and 7 days post-injection. FVIII plasma activity in blood was analyzed by the chromogenic FVIII activity assay.

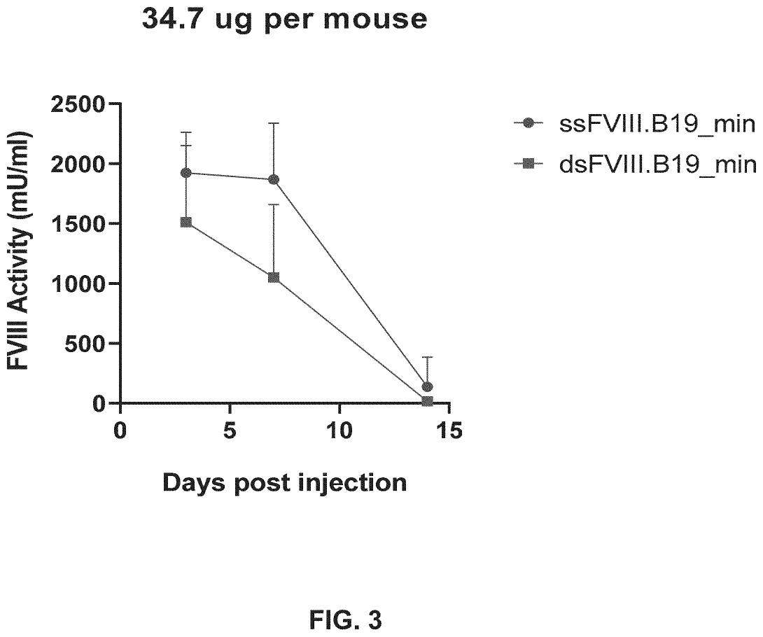

As shown in FIG. 3, analysis of FVIII levels in plasma at 3 days and 7 days post-injection revealed that ssDNA produced higher levels of FVIII than dsDNA. At day 7, FVIII levels from ssDNA remained stable, but FVIII levels from dsDNA had decreased by about 30%. At day 14, FVIII levels were near zero for both ssDNA and dsDNA. ELISA of the samples at day 14 revealed the presence of anti-FVIII antibodies (inhibitors) in all mice. Without being bound to theory, near zero FVIII levels at day 14 may be due to the prescence of the anti-FVIII antibodies, likely formed due to supraphysiological levels of FVIII expression. This example demonstrates that ssDNA and dsDNA constructs containing modified ITRs, such as the B19 ITRs, can express FVIII.

### Example 5: Evaluation of In Vivo Gene Expression from ssDNA Constructs

To validate the functionality of ssDNA constructs comprising the codon optimized FVIII expression cassette flanked by modified ITRs, each ssDNA construct will be tested in a similar manner as the ssFVIII.B19_min construct in hFVIIIR593C^{+/+}/HemA mice (see Example 4). Exemplary ITR sequences are set forth as SEQ ID NOs. 1 to 22 in Table 2. Combinations of GPV and B19 wildtype ITRs and modified ITRs will be generated and tested.

Single stranded DNA from codon optimized FVIII expression constructs with flanking modified ITRs will be generated as in Example 3 and tested in hFVIIIR593C^{+/+}/HemA mice (5-12 weeks of age) for liver directed FVIII expression driven by the mTTR promoter in the codon optimized FVIII expression cassette. HumanFVIIIR593C^{+/+}/HemA mice will be injected via hydrodynamic injection with 10, 20, 37, 50 μg, or other predetermined quantity of ssDNA containing the aforementioned expression cassettes and FVIII will be measured from murine plasma collected at 1, 3, 7, 14, 21, and 28 days post-injection, or other predetermined time intervals. FVIII expression and longevity in mice administered these expression cassettes with modified ITRs will be directly compared with FVIII expression and longevity in mice administered ssDNA constructs with B19Δ135 ITRs, GPVΔ162 ITRs, and/or the corresponding wildtype ITR expression cassettes. FVIII activity in blood will be analyzed by the chromogenic FVIII activity assay.

### Example 6. Use of a Baculovirus Expression System to Generate ceDNA Expression Constructs Bearing Modified ITRs

Systemic delivery of closed-end DNA (ceDNA) expression cassettes has been demonstrated to establish persistent transduction of hepatocytes and drive stable long-term transgene expression in the liver. To test transduction efficiency and gene expression, the baculovirus expression system described in Example 1D of WO2020/033863 can be used for production of FVIII expression for genetic constructs bearing modified ITRs in a form of closed-end DNA (ceDNA) molecules in insect cells. This baculovirus expression system is based on the system described in Li et al., PLoS ONE 8(8): e69879 (2013).

### Example 7. Evaluation of In Vivo Gene Expression from ceDNA Constructs Bearing Modified ITRs in HemA Mice

The ceDNA constructs containing the optimized FVIII expression cassette flanked by a modified 5′ ITR and a modified 3′ ITR will be tested in a similar manner as the pFVIII.B19_min construct in hFVIIIR593C^{+/+}/HemA mice (see Example 4). Exemplary ITR sequences are set forth as SEQ ID NOs. 1 to 22 in Table 2. ceDNA constructs are generated as described in Example 6.

The ceDNA from codon optimized FVIII expression constructs with flanking modified ITRs will be tested in hFVIIIR593C^{+/+}/HemA mice (5-12 weeks of age) for liver directed FVIII expression. HumanFVIIIR593C^{+/+}/HemA mice will be injected via hydrodynamic injection with 10, 20, 37, 50 μg, or other predetermined quantity of ceDNA containing the aforementioned expression cassettes and FVIII will be measured from murine plasma collected at 1, 3, 7, 14, 21, and 28 days post-injection, or other predetermined time intervals. FVIII expression and longevity in mice administered these expression cassettes with modified ITRs will be directly compared with FVIII expression and longevity in mice administered ceDNA constructs with B19Δ135 ITRs, GPVΔ162 ITRs, and/or the corresponding wildtype ITR expression cassettes. FVIII activity in blood will be analyzed by the chromogenic FVIII activity assay.

### Example 8. Generation of Reporter Genetic Constructs Bearing ITRs of B19 or GPV Origin

In order to demonstrate the utility of modified ITR-based genetic expression systems as a platform for general use in gene therapy applications, reporter constructs comprising an expression cassette will be generated with green fluorescent protein (GFP) or luciferase (luc) flanked with a modified 5′ ITR and a modified 3′ ITR. The same procedure as Example 2 is used. However, the open reading frame (ORF) of FVIII in the codon optimized FVIII expression cassette is replaced with either ORF of GFP or luc by conventional molecular cloning techniques.

Expression cassettes flanked by modified ITRs will also be generated which contain the murine phenylalanine hydroxylase (PAH) transgene, which is used to evaluate PAH expression and reduction of blood phenylalanine concentrations in a relevant mouse model of phenylketonuria. Using this model, PKU mice are administered 200 μg of ssDNA flanked by modified ITRs via hydrodynamic injection for liver expression. Blood samples will be collected at days 3, 7, 14, 28, 42, 56, 70, and 81 and plasma will be isolated for phenylalanine concentration determination. To confirm the presence of murine PAH protein in the liver, a Western blot will be performed on liver lysates taken from treated mice at day 81 post injection.

### Example 9. Preparation of ssDNA Reporter Genetic Constructs Bearing Modified ITRs

ssDNA reporter or PAH constructs will be prepared as described in Example 3. Briefly, plasmids will be digested with LguI, MscI, and Eco53kl restriction enzymes. ssDNA fragments with formed hairpin ITR structures will be generated by denaturing the double-stranded DNA fragment products (reporter expression cassette and plasmid backbone) of the restriction enzyme digestion at 95° C. and then cooling down at 4° C. to allow the palindromic ITR sequences to fold. The resulting ssDNA constructs can be tested in mice for the ability to establish persistent transduction of liver, muscle tissue, photoreceptors in the eye, central nervous system (CNS), or other tissues by detection of the reporter gene or PAH.

### Example 10. In Vivo Evaluation of ssDNA-Mediated Reporter Expression

To validate the ability of the ssDNA reporter constructs described in Example 13 to mediate persistent transgene expression in vivo, 5-12-week old mice (at least 4 animals/group) will be injected with 5, 10, or 20 μg/mouse of reporter ssDNA systemically and/or locally to the target tissue. Blood samples are collected at predetermined time points and levels of reporter transgene can be detected and/or measured using conventional techniques.

### Example 11: Modified V2.0 FVIIIXTEN Expression Cassette with Engineered Parvoviral ITRs

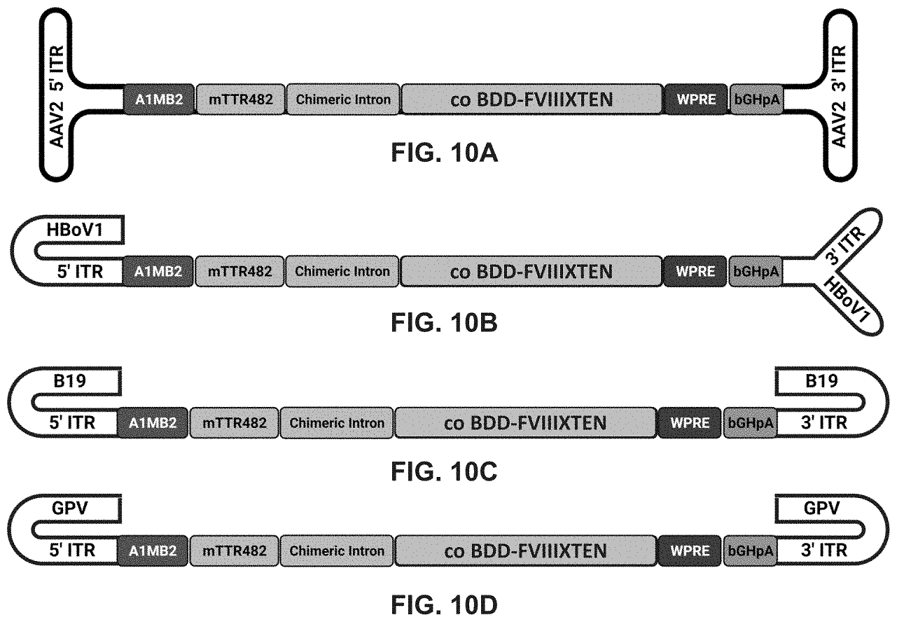

It was hypothesized that the transgene expression level can be increased by codon-optimizing cDNA for the targeted hosts. The physiological levels of FVIII expression from V1.0 FVIIIco6XTEN expression cassette have been demonstrated in previous studies as described in U.S. Publication No. 20190185543. However, to further improve the transgene expression level and reduce the immunogenicity, the FVIIIXTEN cDNA was codon-optimized with CpG repeats depleted to escape innate immune response raised against the DNA vector encoding FVIIIXTEN expression cassette with parvoviral ITRs. The modified V2.0 FVIIIXTEN expression cassette was generated which comprises a B-domain deleted (BDD) codon-optimized human Factor VIII (BDDcoFVIII) fused with XTEN 144 peptide (FVIIIXTEN) under the regulation of liver-specific modified mouse transthyretin (mTTR) promoter (mTTR482) with enhancer element (A1MB2), hybrid synthetic intron (Chimeric Intron), the Woodchuck Posttranscriptional Regulatory Element (WPRE), and the Bovine Growth Hormone Polyadenylation (bGHpA) signal. The V2.0 FVIIIXTEN expression cassette comprises the nucleotide sequence of SEQ ID NO: 27. Graphical depictions of exemplary V2.0 FVIIIXTEN expression cassettes are shown in FIG. 10.

Initial in vivo efficacy studies showed significant improvement in FVIII activity in comparison with the V1.0 FVIIIXTEN expression cassette (data not shown). Therefore, engineered parvoviral ITRs including AAV2 WT (FIG. 10A), HBoV1 WT (SEQ ID NO: 25, SEQ ID NO: 26) (FIG. 10B), B19 WT (SEQ ID NO: 23, SEQ ID NO: 24), B19 Minimal (SEQ ID: 1, SEQ ID: 2) (FIG. 10C), and GPVΔ186 (SEQ ID: 9, SEQ ID: 10), GPVΔ120 (SEQ ID NO: 13, SEQ ID NO: 14), or GPV Minimal (SEQ ID NO: 15, SEQ ID NO: 16) ITRs (FIG. 10D) were cloned such that the ITRs were flanking the V2.0 FVIIIXTEN expression cassette. The in vivo functionality of the modified V2.0 FVIIIXTEN expression cassette was also demonstrated with different parvoviral ITRs in a form of single-stranded (ss) or closed-end (ce) DNA by systemic administration via hydrodynamic tail-vein injections in hFVIIIR593C^{+/+}/HemA mice, as shown below.

### Example 12: In Vivo Evaluation of Single-Stranded FVIIIXTEN (ssFVIIIXTEN) DNA

It was hypothesized that the hairpin formed within the ITR region drives the long-term persistent expression of transgene at higher level. To validate the functionality of modified FVIIIXTEN expression cassette in vivo, single-stranded DNA (ssDNA) comprising the V2.0 FVIIIXTEN genetic cassette with engineered parvoviral ITRs was tested in hFVIIIR593C^{+/+}/HemA mice. These mice contain a human FVIII-R593C transgene, designed with the murine albumin (Alb) promoter driving expression of an altered human coagulation factor VIII (FVIII) cDNA harboring a mutation that is frequently observed in patients with mild hemophilia A. These mice also carry a knock-out of the FVIII gene and are deficient for endogenous FVIII protein. These double mutant mice are tolerant of human FVIII injection and have no FVIII activity. They produce very little inhibitory antibodies and lack FVIII responsive T cells or B cells after treatment with human FVIII. The hFVIIIR593C^{+/+}/HemA mouse is further described in Bril, et al. (2006) Thromb. Haemost. 95(2): 341-7.

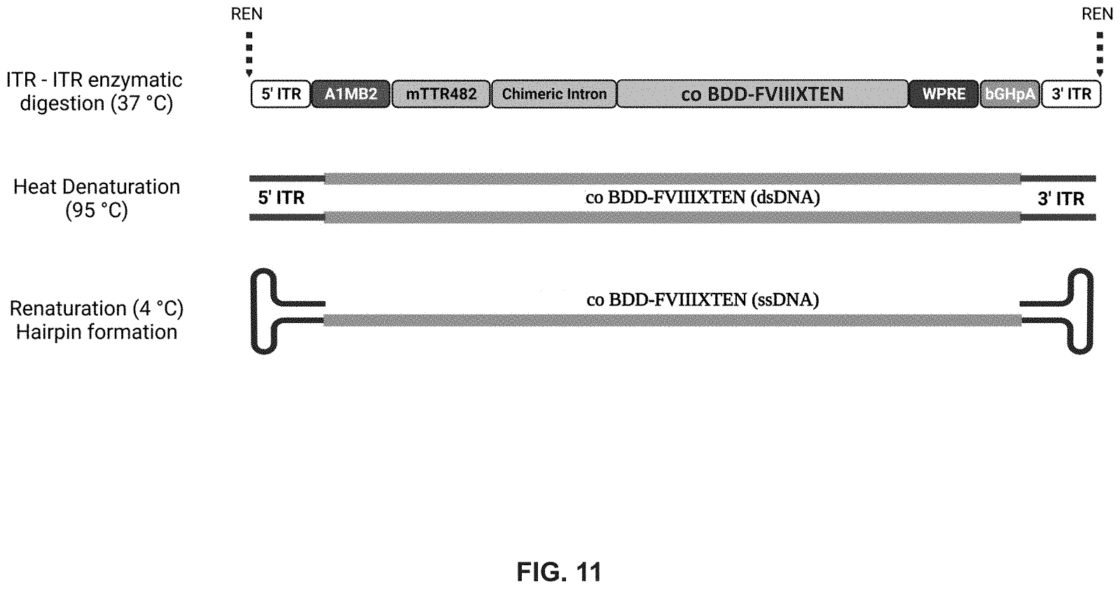

The ssFVIIIXTEN with different parvoviral ITRs was generated by digesting the plasmid DNA constructs with restriction enzymes that recognize the ITR related sequence and produce blunt-end DNA. The digested double-stranded DNA products (FVIII expression cassette and plasmid backbone) were heat denatured (denaturation) at 95° C. followed by cooling (renaturation) at 4° C. to allow the palindromic ITR sequences to form hairpins (FIG. 11). The resulting ssFVIIIXTEN (ssDNA) was then systemically administered via hydrodynamic tail-vein injections at 200, 800, or 1600 μg/kg in hFVIIIR593C^{+/+}/HemA mice. Plasma samples were collected from injected mice at indicated intervals for 5.5 months and the FVIII activity was measured by the Chromogenix Coatest® SP Factor VIII chromogenic assay, according to the manufacturer's instructions.

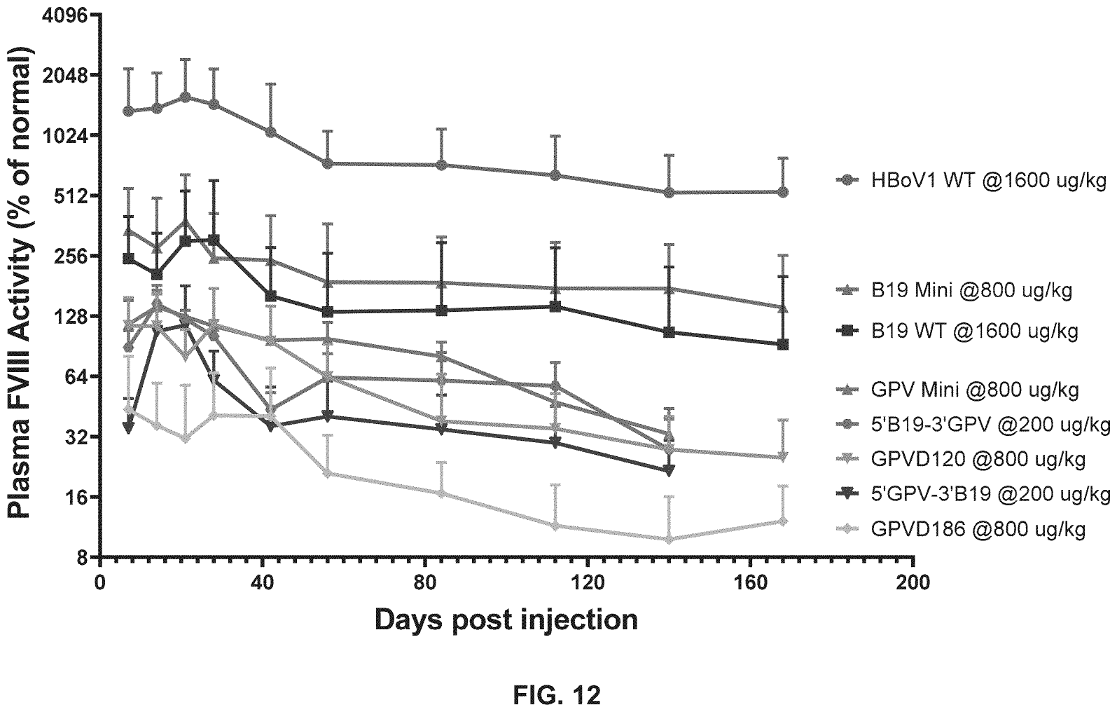

The plasma FVIII activity normalized to the percent of normal for ssFVIIIXTEN injected animals is shown in FIG. 12. The results showed long-term persistent FVIIIXTEN expression in all the parvoviral ITRs tested albeit with varying levels. All variants of GPV or hybrid GPV ITRs tested showed continuous decline in the levels of FVIIIXTEN expression in comparison with other parvoviral ITRs. In contrary, HBoV1 and B19 ITRs showed initial decline in FVIIIXTEN up to day 56 and then stabilized through day 168, suggesting the ITR-dependent persistency of the FVIIIXTEN transgene in vivo. Unlike GPV ITRs, both B19 and HBoV1 ITRs showed significantly higher levels of FVIII expression irrespective of the variant tested. Among different parvoviral ITRs tested, HBoV1 ITRs showed significantly higher levels (>1000%) of normal FVIII activity in hFVIIIR593C^{+/+}/HemA mice. (FIG. 10B). These results validate the functionality of the modified FVIIIXTEN expression cassette with different parvoviral ITRs and demonstrate the ITR-dependent stability as well as persistency of transgene expression in vivo.

### Example 13: In Vivo Evaluation of Closed-End FVIIIXTEN (ceFVIIIXTEN) DNA

Though ssFVIIIXTEN (ssDNA) was effective in expressing a modified FVIIIXTEN expression cassette in vivo, there are several limitations associated with ssDNA to be used as a non-viral gene therapy vector. One of them is the level of endotoxin contamination due to the prokaryotic host (E. coli) used for generating plasmid DNA, which also contains the extraneous sequences, such as antibiotic resistance gene and prokaryotic origin of replication, needed for selection and amplification in E. coli. To address these challenges and limitations, a eukaryotic cell-based system was developed to generate DNA therapeutic drug substance in a form of closed-end DNA (ceDNA) comprising of the FVIIIXTEN expression cassette with parvoviral ITRs. The genetic organization of ceDNA, resembles recombinant AAV vector DNA, but differs in conformation.

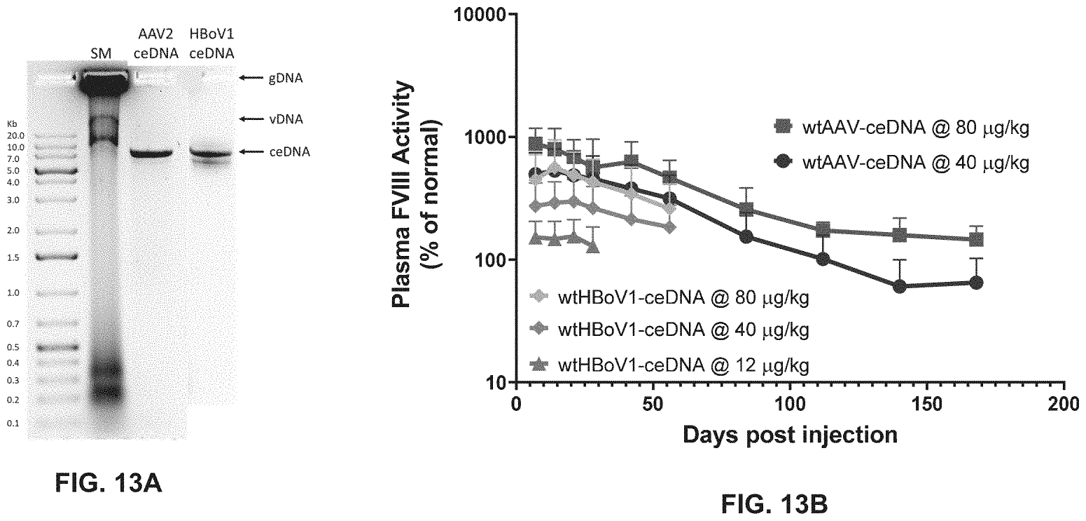

To generate this DNA vector, the baculovirus insect cell system was leveraged, which is widely used for the biologics manufacturing and the only platform approved by the FDA for influenza vaccine manufacturing. Three different approaches of ceDNA production were employed in the baculovirus system, as described in U.S. Patent Application No. 63/069,073. An exemplary agarose gel image of the purified ceDNA encoding modified V2.0 FVIIIXTEN flanked by the AAV2 WT or HBoV1 WT ITRs in comparison with the starting material (SM) is shown in FIG. 13A.

To validate the functionality of modified FVIIIXTEN as expressed from ceDNA, purified ceFVIIIXTEN was injected systemically via hydrodynamic tail-vein injections in hFVIIIR593C^{+/+}/HemA mice at 0.3 μg, 1.0 μg, or 2.0 μg/mouse, which is equivalent to 12 μg, 40 μg, and 80 μg/kg, respectively. Plasma samples from injected mice were collected at indicated intervals and FVIII activity was measured by the chromogenic assay, as described above.

The plasma FVIII activity normalized to the percent of normal for ceFVIIIXTEN injected animals is shown in FIG. 13B. The results showed dose-dependent response in HemA mice with supraphysiological levels (>500% of normal) of FVIII expression observed in the highest dose of AAV2 or HBoV1 ceDNA tested. However, a gradual decline in FVIII expression levels was observed upto day 140 post injections, after which the levels were stabilized in ceFVIIIXTEN AAV2 ITRs injected cohorts. Interestingly, the FVIII expression levels observed for ceFVIIIXTEN HBoV1 ITRs showed similar trend as observed in the cohorts injected with ceFVIIIXTEN AAV2 ITRs.

These in vivo efficacy studies validate the functionality of ceFVIIIXTEN DNA and proves the concept that parvoviral ITRs can be used to produce functional non-viral gene therapy vectors encoding transgenes of interest in the baculovirus insect cell system.

### Sequences

TABLE 2
Additional sequences

| SEQ ID NO and | |
|---|---|
| description | Nucleotide or Amino Acid Sequence |
| SEQ ID NO. 1: | CCAAATCAGATGCCGCCGGTCGCCGCCGGTAGGCGGGACTTCCGGTACAGCGCGCCGCTGTACCGGAAGTCCCG |
| 5′_B19_min | CCTACCGGCGGCGACCGGCGGCATCTGATTTGGTGTCTTCTTTTAAATTTT |
| SEQ ID NO. 2: | AAAATTTAAAAGAAGACACCAAATCAGATGCCGCCGGTCGCCGCCGGTAGGCGGGACTTCCGGTACAGCGGCGC |
| 3′_B19_min | GCTGTACCGGAAGTCCCGCCTACCGGCGGCGACCGGCGGCATCTGATTTGG |
| SEQ ID NO. 3: | CCAAATCAGATGCCGCCGGTCGCCGCCGGTAGGCGGGACTTCCGGTACAAGATGGCGGACAATTAGCGGCGCGC |
| 5′_B19_Δ223 | CGCTAATTGTCCGCCATCTTGTACCGGAAGTCCCGCCTACCGGCGGCGACCGGCGGCATCTGATTTGGTGTCTT |
| | CTTTTAAATTTT |
| SEQ ID NO. 4: | AAAATTTAAAAGAAGACACCAAATCAGATGCCGCCGGTCGCCGCCGGTAGGCGGGACTTCCGGTACAAGATGGC |
| 3′_B19_Δ223 | GGACAATTAGCGGCGCGCCGCTAATTGTCCGCCATCTTGTACCGGAAGTCCCGCCTACCGGCGGCGACCGGCGG |
| | CATCTGATTTGG |
| SEQ ID NO. 5: | CCAAATCAGATGCCGCCGGTCGCCGCCGGTAGGCGGGACTTCCGGTACAAGATGGCGGACAATTACGTCATTTC |
| 5′_B19_Δ151 | CTGTGACGTCATTTCCTGTGACGTCACGCGGCGCGCCGCGTGACGTCACAGGAAATGACGTCACAGGAAATGAC |
| | GTAATTGTCCGCCATCTTGTACCGGAAGTCCCGCCTACCGGCGGCGACCGGCGGCATCTGATTTGGTGTCTTCT |
| | TTTAAATTTT |
| SEQ ID NO. 6: | AAAATTTAAAAGAAGACACCAAATCAGATGCCGCCGGTCGCCGCCGGTAGGCGGGACTTCCGGTACAAGATGGC |
| 3′_B19_Δ151 | GGACAATTACGTCATTTCCTGTGACGTCATTTCCTGTGACGTCACGCGGCGCGCCGCGTGACGTCACAGGAAAT |
| | GACGTCACAGGAAATGACGTAATTGTCCGCCATCTTGTACCGGAAGTCCCGCCTACCGGCGGCGACCGGCGGCA |
| | TCTGATTTGG |
| SEQ ID NO. 7: | CTCTGGGCCAGCTTGCTTGGGGTTGCCTTGACACTAAGACAAGCGGCGCGCCGCTTGATCTTAGTGGCACGTCA |
| 5′_B19_Δ135 | ACCCCAAGCGCTGGCCCAGAGCCAACCCTAATTCCGGAAGTCCCGCCCACCGGAAGTGACGTCACAGGAAATGA |
| | CGTCACAGGAAATGACGTAATTGTCCGCCATCTTGTACCGGAAGTCCCGCCTACCGGCGGCGACCGGCGGCATC |
| | TGATTTGGTGTCTTCTTTTAAATTTT |
| SEQ ID NO. 8: | AAAATTTAAAAGAAGACACCAAATCAGATGCCGCCGGTCGCCGCCGGTAGGCGGGACTTCCGGTACAAGATGGC |
| 3′_B19_Δ135 | GGACAATTACGTCATTTCCTGTGACGTCATTTCCTGTGACGTCACTTCCGGTGGGCGGGACTTCCGGAATTAGG |
| | GTTGGCTCTGGGCCAGCGCTTGGGGTTGACGTGCCACTAAGATCAAGCGGCGCGCCGCTTGTCTTAGTGTCAAG |
| | GCAACCCCAAGCAAGCTGGCCCAGAG |
| SEQ ID NO. 9: | CTCATTGGAGGGTTCGTTCGTTCGAACCAGCCAATCAGGGGAGGGGGAAGTGACGCAAGTTCCGGTCACATGCT |
| 5′_GPV_Δ186 | TCCGGTGACGCACATCCGGTGACGTAGTTCGCATGCGAACTACGTCACCGGATGTGCGTCACCGGAAGCATGTG |
| | ACCGGAACTTGCGTCACTTCCCCCTCCCCTGATTGGCTGGTTCGAACGAACGAACCCTCCAATGAGACTCAAGG |
| | ACAAGAGGATATTTTGCGCGCCAGGAAGTG |
| SEQ ID NO. 10: | CACTTCCTGGCGCGCAAAATATCCTCTTGTCCTTGAGTCTCATTGGAGGGTTCGTTCGTTCGAACCAGCCAATC |
| 3′_GPV_Δ186 | AGGGGAGGGGGAAGTGACGCAAGTTCCGGTCACATGCTTCCGGTGACGCACATCCGGTGACGTAGTTCGCATGC |
| | GAACTACGTCACCGGATGTGCGTCACCGGAAGCATGTGACCGGAACTTGCGTCACTTCCCCCTCCCCTGATTGG |
| | CTGGTTCGAACGAACGAACCCTCCAATGAG |
| SEQ ID NO. 11: | CGGTGACGTGTTTCCGGCTGTTAGGTTGACCACGCGCATGCCGCGCGGTCAGCCCAATAGTTAAGCCGGAAACA |
| 5′_GPV_Δ162 | CGTCACCGGAAGTCACATGACCGGAAGTCACGTGACCGGAAACACGTGACAGGAAGCACGTGACCGGAACTACG |
| | TCACCGGATGTGCGTCACCGGAAGCATGTGACCGGAACTTGCGTCACTTCCCCCTCCCCTGATTGGCTGGTTCG |
| | AACGAACGAACCCTCCAATGAGACTCAAGGACAAGAGGATATTTTGCGCGCCAGGAAGTG |
| SEQ ID NO. 12: | CACTTCCTGGCGCGCAAAATATCCTCTTGTCCTTGAGTCTCATTGGAGGGTTCGTTCGTTCGAACCAGCCAATC |
| 3′_GPV_Δ162 | AGGGGAGGGGGAAGTGACGCAAGTTCCGGTCACATGCTTCCGGTGACGCACATCCGGTGACGTAGTTCCGGTCA |
| | CGTGCTTCCTGTCACGTGTTTCCGGTCACGTGACTTCCGGTCATGTGACTTCCGGTGACGTGTTTCCGGCTTAA |
| | CTATTGGGCTGACCGCGCGGCATGCGCGTGGTCAACCTAACAGCCGGAAACACGTCACCG |
| SEQ ID NO. 13: | CTCATTGGAGGGTTCGTTCGTTCGAACCAGCCAATCAGGGGAGGGGGAAGTGACGCAAGTTCCGGTCACATGCT |
| 5′_GPV_Δ120 | TCCGGTGACGCACATCCGGTGACGTAGTTCCGGTCACGTGCTTCCTGTCACGTGTTTCCGGTCGCATGCTCACG |
| | TGACCGGAAACACGTGACAGGAAGCACGTGACCGGAACTACGTCACCGGATGTGCGTCACCGGAAGCATGTGAC |
| | CGGAACTTGCGTCACTTCCCCCTCCCCTGATTGGCTGGTTCGAACGAACGAACCCTCCAATGAGACTCAAGGAC |
| | AAGAGGATATTTTGCGCGCCAGGAAGTG |
| SEQ ID NO. 14: | CACTTCCTGGCGCGCAAAATATCCTCTTGTCCTTGAGTCTCATTGGAGGGTTCGTTCGTTCGAACCAGCCAATC |
| 3′_GPV_Δ120 | AGGGGAGGGGGAAGTGACGCAAGTTCCGGTCACATGCTTCCGGTGACGCACATCCGGTGACGTAGTTCCGGTCA |
| | CGTGCTTCCTGTCACGTGTTTCCGGTCACGTGAGCATGCGACCGGAAACACGTGACAGGAAGCACGTGACCGGA |
| | ACTACGTCACCGGATGTGCGTCACCGGAAGCATGTGACCGGAACTTGCGTCACTTCCCCCTCCCCTGATTGGCT |
| | GGTTCGAACGAACGAACCCTCCAATGAG |
| SEQ ID NO. 15: | CTCATTGGAGGGTTCGTTCGTTCGAACGTTCGTTCGCATGCGAACGAACGTTCGAACGAACGAACCCTCCAATG |
| 5′_GPV_minimal | AGACTCAAGGACAAGAGGATATTTTGCGCGCCAGGAAGTG |
| SEQ ID NO. 16: | CACTTCCTGGCGCGCAAAATATCCTCTTGTCCTTGAGTCTCATTGGAGGGTTCGTTCGTTCGAACGTTCGTTCG |
| 3′_GPV_minimal | CATGCGAACGAACGTTCGAACGAACGAACCCTCCAATGAG |
| SEQ ID NO. 17: | CCAAATCAGATGCCGCCGGTCGCCGCCGGTAGGCGGGACTTCCGGTACAAGATGGCGGACAATTACGTCATTTC |
| 5′_B19_Δ151_ | CTGTGACGTCATTTCCTGTGACGTCACGCGGCGCGCCTATTGCTTGGCAGCGCCTATCGCCAGGTATTACTCCA |
| extended_loop | ATCCCGAATATCCGAGATCGGGATCACCCGAGAGAAGTTCAACCTACATCCTCAATCCCGATCTATCCGAGGCG |
| | CGCCGCGTGACGTCACAGGAAATGACGTCACAGGAAATGACGTAATTGTCCGCCATCTTGTACCGGAAGTCCCG |
| | CCTACCGGCGGCGACCGGCGGCATCTGATTTGGTGTCTTCTTTTAAATTTT |
| SEQ ID NO. 18: | AAAATTTAAAAGAAGACACCAAATCAGATGCCGCCGGTCGCCGCCGGTAGGCGGGACTTCCGGTACAAGATGGC |
| 3′_B19_Δ151_ | GGACAATTACGTCATTTCCTGTGACGTCATTTCCTGTGACGTCACGCGGCGCGCCAGCGCCTATCGCCAGGTAT |
| extended_loop | TACTCCAATCCCGAATATCCGAGATCGGGATCACCCGAGAGAAGTTCAACCTACATCCTCAATCCCGATCTATC |
| | CGAGATCCGGCGCGCCGCGTGACGTCACAGGAAATGACGTCACAGGAAATGACGTAATTGTCCGCCATCTTGTA |
| | CCGGAAGTCCCGCCTACCGGCGGCGACCGGCGGCATCTGATTTGG |
| SEQ ID NO. 19: | CTCATTGGAGGGTTCGTTCGTTCGAACCAGCCAATCAGGGGAGGGGGAAGTGACGCAAGTTCCGGTCACATGCT |
| 5′_GPV_Δ186_ | TCCGGTGACGCACATCCGGTGACGTAGTTCGCATGCCTGTCTATCGCCTACCCATCCCTGTCTGAGATCAAGGG |
| extended_loop | CGTGATCGTGCACAGACTGGAGAGCGTGTCCTATAATATCGGCTCTCAGGAGTGGAGCACCACAGTGCCCAGAT |
| | ACGTGGCCACCCAGGGCTATCTGATCTCCAACTTCGACGCATGCGAACTACGTCACCGGATGTGCGTCACCGGA |
| | AGCATGTGACCGGAACTTGCGTCACTTCCCCCTCCCCTGATTGGCTGGTTCGAACGAACGAACCCTCCAATGAG |
| | ACTCAAGGACAAGAGGATATTTTGCGCGCCAGGAAGTG |
| SEQ ID NO. 20: | CACTTCCTGGCGCGCAAAATATCCTCTTGTCCTTGAGTCTCATTGGAGGGTTCGTTCGTTCGAACCAGCCAATC |
| 3′_GPV_Δ186_ | AGGGGAGGGGGAAGTGACGCAAGTTCCGGTCACATGCTTCCGGTGACGCACATCCGGTGACGTAGTTCGCATGC |
| extended_loop | CTGTCTATCGCCTACCCATCCCTGTCTGAGATCAAGGGCGTGATCGTGCACAGACTGGAGAGCGTGTCCTATAA |
| | TATCGGCTCTCAGGAGTGGAGCACCACAGTGCCCAGATACGTGGCCACCCAGGGCTATCTGATCTCCAACTTCG |
| | ACGCATGCGAACTACGTCACCGGATGTGCGTCACCGGAAGCATGTGACCGGAACTTGCGTCACTTCCCCCTCCC |
| | CTGATTGGCTGGTTCGAACGAACGAACCCTCCAATGAG |
| SEQ ID NO. 21: | CTCATTGGAGGGTTCGTTCGTTCGAACCAGCCAATCAGGGGAGGGGGAAGTGACGCAAGTTCCGGTCACATGCT |
| 5′_GPV_Δ120_ | TCCGGTGACGCACATCCGGTGACGTAGTTCCGGTCACGTGCTTCCTGTCACGTGTTTCCGGTCGCATGCCTGTC |
| extended_loop | TATCGCCTACCCATCCCTGTCTGAGATCAAGGGCGTGATCGTGCACAGACTGGAGAGCGTGTCCTATAATATCG |
| | GCTCTCAGGAGTGGAGCACCACAGTGCCCAGATACGTGGCCACCCAGGGCTATCTGATCTCCAACTTCGACGCA |
| | TGCTCACGTGACCGGAAACACGTGACAGGAAGCACGTGACCGGAACTACGTCACCGGATGTGCGTCACCGGAAG |
| | CATGTGACCGGAACTTGCGTCACTTCCCCCTCCCCTGATTGGCTGGTTCGAACGAACGAACCCTCCAATGAGAC |
| | TCAAGGACAAGAGGATATTTTGCGCGCCAGGAAGTG |
| SEQ ID NO. 22: | CACTTCCTGGCGCGCAAAATATCCTCTTGTCCTTGAGTCTCATTGGAGGGTTCGTTCGTTCGAACCAGCCAATC |
| 3′_GPV_Δ120_ | AGGGGAGGGGGAAGTGACGCAAGTTCCGGTCACATGCTTCCGGTGACGCACATCCGGTGACGTAGTTCCGGTCA |
| extended_loop | CGTGCTTCCTGTCACGTGTTTCCGGTCACGTGAGCATGCCTGTCTATCGCCTACCCATCCCTGTCTGAGATCAA |
| | GGGCGTGATCGTGCACAGACTGGAGAGCGTGTCCTATAATATCGGCTCTCAGGAGTGGAGCACCACAGTGCCCA |
| | GATACGTGGCCACCCAGGGCTATCTGATCTCCAACTTCGACGGCATGCGACCGGAAACACGTGACAGGAAGCAC |
| | GTGACCGGAACTACGTCACCGGATGTGCGTCACCGGAAGCATGTGACCGGAACTTGCGTCACTTCCCCCTCCCC |
| | TGATTGGCTGGTICGAACGAACGAACCCICCAATGAG |
| SEQ ID NO. 23: | CCAAATCAGATGCCGCCGGTCGCCGCCGGTAGGCGGGACTTCCGGTACAAGATGGCGGACAATTACGTCATTTC |
| B19 WT 5′ | CTGTGACGTCATTTCCTGTGACGTCACTTCCGGTGGGCGGGACTTCCGGAATTAGGGTTGGCTCTGGGCCAGCT |
| | TGCTTGGGGTTGCCTTGACACTAAGACAAGCGGCGCGCCGCTTGATCTTAGTGGCACGTCAACCCCAAGCGCTG |
| | GCCCAGAGCCAACCCTAATTCCGGAAGTCCCGCCCACCGGAAGTGACGTCACAGGAAATGACGTCACAGGAAAT |
| | GACGTAATTGTCCGCCATCTTGTACCGGAAGTCCCGCCTACCGGCGGCGACCGGCGGCATCTGATTTGGTGTCT |
| | TCTTTTAAATTTT |
| SEQ ID NO. 24: | AAAATTTAAAAGAAGACACCAAATCAGATGCCGCCGGTCGCCGCCGGTAGGCGGGACTTCCGGTACAAGATGGC |
| B19 WT 3′ | GGACAATTACGTCATTTCCTGTGACGTCATTTCCTGTGACGTCACTTCCGGTGGGCGGGACTTCCGGAATTAGG |
| | GTTGGCTCTGGGCCAGCGCTTGGGGTTGACGTGCCACTAAGATCAAGCGGCGCGCCGCTTGTCTTAGTGTCAAG |
| | GCAACCCCAAGCAAGCTGGCCCAGAGCCAACCCTAATTCCGGAAGTCCCGCCCACCGGAAGTGACGTCACAGGA |
| | AATGACGTCACAGGAAATGACGTAATTGTCCGCCATCTTGTACCGGAAGTCCCGCCTACCGGCGGCGACCGGCG |
| | GCATCTGATTTGG |
| SEQ ID NO: 25 | TTGTTGTTGTACATGCGCCATCTTAGTTTTATATCAGCTGGCGCCTTAGTTATATAACATGCATGTTATATAACTAAG |
| HBoV1 WT ITR 5′ | GCGCCAGCTGATATAAAACTAAGATGGCGCATGTACAACAACAACACATTAAAAGATATAGAGTTTCGCGATTGC |
| SEQ ID NO: 26 | TATATGTGACGTGGTTGTACAGACGCCATCTTGGAATCCAATATGTCTGCCGGCGATTAGATCATGCGCGCGCGCAGC |
| HBoV1 WT ITR 3′ | GCGCTGCGCGCAGCGCAGGCATGACTGAGCCGGCAGACATATTGGATTCCAAGATGGCGTCTGTACAACCAC |
| SEQ ID NO: 27 | GGCCCCAGGTTAATTTTTAAAAAGCAGTCAAAGGTCAAAGTGGCCCTTGGCAGCATTTACTCTCTCTATTGACTTTGG |
| V2.0 | TTAATAATCTCAGGAGCACAAACATTCCTGGAGGCAGGAGAAGAAATCAACATCCTGGACTTATCCTCTGGGCCTCTC |
| Expression | CCCACCTTCGATGGCCCCAGGTTAATTTTTAAAAAGCAGTCAAAGGTCAAAGTGGCCCTTGGCAGCATTTACTCTCTC |
| cassette | TATTGACTTTGGTTAATAATCTCAGGAGCACAAACATTCCTGGAGGCAGGAGAAGAAATCAACATCCTGGACTTATCC |
| mTTR482-Intron- | TCTGGGCCTCTCCCCACCGATATCTACCTGCTGATCGCCCGGCCCCTGTTCAAACATGTCCTAATACTCTGTCGGGGC |
| coBDDFVIIIXTEN | AAAGGTCGGCAGTAGTTTTCCATCTTACTCAACATCCTCCCAGTGTACGTAGGATCCTGTCTGTCTGCACATTTCGTA |
| (V2.0)-WPRE- | GAGCGAGTGTTCCGATACTCTAATCTCCCGGGGCAAAGGTCGTATTGACTTAGGTTACTTATTCTCCTTTTGTTGACT |
| bGHPolyA | AAGTCAATAATCAGAATCAGCAGGTTTGGAGTCAGCTTGGCAGGGATCAGCAGCCTGGGTTGGAAGGAGGGGGTATAA |
| | AAGCCCCTTCACCAGGAGAAGCCGTCACACAGATCCACAAGCTCCTGCTAGGAATTCTCAGGAGCACAAACATTCCTG |
| | GAGGCAGGAGAAGAAATCAACATCCTGGACTTATCCTCTGGGCCTCTCCCCACCGATATCTACCTGCTGATCGCCCGG |
| | CCCCTGTTCAAACATGTCCTAATACTCTGTCGGGGCAAAGGTCGGCAGTAGTTTTCCATCTTACTCAACATCCTCCCA |
| | GTGTACGTAGGATCCTGTCTGTCTGCACATTTCGTAGAGCGAGTGTTCCGATACTCTAATCTCCCGGGGCAAAGGTCG |
| | TATTGACTTAGGTTACTTATTCTCCTTTTGTTGACTAAGTCAATAATCAGAATCAGCAGGTTTGGAGTCAGCTTGGCA |
| | GGGATCAGCAGCCTGGGTTGGAAGGAGGGGGTATAAAAGCCCCTTCACCAGGAGAAGCCGTCACACAGATCCACAAGC |
| | TCCTGCTAGAGTCGCTGCGCGCTGCCTTCGCCCCGTGCCCCGCTCCGCCGCCGCCTCGCGCCGCCCGCCCCGGCTCTG |
| | ACTGACCGCGTTACTCCCACAGGTGAGCGGGCGGGACGGCCCTTCTCCTCCGGGCTGTAATTAGCGCTTGGTTTATTG |
| | ACGGCTTGTTTCTTTTCTGTGGCTGCGTGAAAGCCTTGAGGGGCTCCGGGAAGGCCCTTTGTGCGGGGGGAGCGGCTC |
| | GGGGGGTGCGTGCGTGTGTGTGTGCGTGGGGAGCGCCGCGTGCGGCTCCGCGCTGCCCGGCGGCTGTGAGCGCTGCGG |
| | GCGCGGCGCGGGGCTTTGTGCGCTCCGCAGTGTGCGCGAGGGGAGCGCGGCCGGGGGCGGTGCCCCGCGGTGCGGGGG |
| | GGGCTGCGAGGGGAACAAAGGCTGCGTGCGGGGTGTGTGCGTGGGGGGGTGAGCAGGGGGTGTGGGCGCGTCGGTCGG |
| | GCTGCAACCCCCCCTGCACCCCCCTCCCCGAGTTGCTGAGCACGGCCCGGCTTCGGGTGCGGGGCTCCGTACGGGGCG |
| | TGGCGCGGGGCTCGCCGTGCCGGGCGGGGGGTGGCGGCAGGTGGGGGTGCCGGGCGGGGCGGGGCCGCCTCGGGCCGG |
| | GGAGGGCTCGGGGGAGGGGCGCGGCGGCCCCCGGAGCGCCGGCGGCTGTCGAGGCGCGGCGAGCCGCAGCCATTGCCT |
| | TTTATGGTAATCGTGCGAGAGGGCGCAGGGACTTCCTTTGTCCCAAATCTGTGCGGAGCCGAAATCTGGGAGGCGCCG |
| | CCGCACCCCCTCTAGCGGGCGCGGGGCGAAGCGGTGCGGCGCCGGCAGGAAGGAAATGGGCGGGGAGGGCCTTCGTGC |
| | GTCGCCGCGCCGCCGTCCCCTTCTCCCTCTCCAGCCTCGGGGCTGTCCGCGGGGGGACGGCTGCCTTCGGGGGGGACG |
| | GGGCAGGGCGGGGTTCGGCTTCTGGCGTGTGACCGGCGGCTCTAGAGCCTCTGCTAACCTTGTTCTTGCCTTCTTCTT |
| | TTTCCTACAGCTCCTGGGCAACGTGCTGGTTATTGTGCTGTCTCATCATTTTGGCAAAGAATTACTCGAGGCCACCAT |
| | GCAGATTGAACTGTCCACTTGCTTCTTCCTGTGCCTCCTGCGGTTTTGCTTCTCGGCCACCCGCCGGTATTACTTAGG |
| | TGCTGTGGAACTGAGCTGGGACTACATGCAGTCCGACCTGGGAGAACTGCCGGTGGACGCGAGATTCCCACCTAGAGT |
| | CCCGAAGTCCTTCCCATTCAACACCTCCGTGGTCTACAAAAAGACCCTGTTCGTGGAGTTCACTGACCACCTTTTCAA |
| | TATTGCCAAGCCGCGCCCCCCCTGGATGGGCCTGCTTGGTCCTACGATCCAAGCAGAGGTCTACGACACCGTGGTCAT |
| | CACACTGAAGAACATGGCCTCACACCCCGTGTCGCTGCATGCTGTGGGAGTGTCCTACTGGAAGGCCTCAGAGGGTGC |
| | CGAATATGATGACCAGACCAGCCAGAGGGAAAAGGAGGATGACAAAGTGTTCCCGGGTGGCAGCCACACTTACGTGTG |
| | GCAAGTGCTGAAGGAAAACGGGCCTATGGCGTCGGACCCCCTATGCCTGACCTACTCCTACCTGTCCCATGTGGACCT |
| | TGTGAAGGATCTCAACTCGGGACTGATCGGCGCCCTCTTGGTGTGCAGAGAAGGCAGCCTGGCGAAGGAAAAGACTCA |
| | GACCCTGCACAAGTTCATTCTGTTGTTTGCTGTGTTCGATGAAGGAAAGTCCTGGCACTCAGAAACCAAGAACTCGCT |
| | GATGCAGGATAGAGATGCGGCCTCGGCCAGAGCCTGGCCTAAAATGCACACCGTCAACGGATATGTGAACAGGTCGCT |
| | CCCTGGCCTCATCGGCTGCCACAGAAAGTCCGTGTATTGGCATGTGATCGGCATGGGTACTACTCCGGAAGTGCATAG |
| | TATCTTTCTGGAGGGCCATACCTTCTTGGTGCGCAACCACAGACAGGCCTCGCTGGAAATCTCGCCTATCACTTTCTT |
| | GACTGCGCAGACCCTCCTTATGGACCTTGGACAGTTCCTGCTGTTCTGTCACATCAGCTCCCATCAGCATGATGGGAT |
| | GGAGGCCTATGTCAAAGTGGACTCCTGCCCTGAGGAGCCACAGCTCCGGATGAAGAACAATGAGGAAGCGGAGGATTA |
| | CGACGACGACCTGACTGACAGCGAAATGGACGTCGTGCGATTCGATGACGACAACAGCCCGTCCTTCATCCAAATTAG |
| | ATCAGTGGCGAAGAAGCACCCCAAGACCTGGGTGCACTACATTGCCGCCGAGGAAGAGGACTGGGACTACGCGCCGCT |
| | GGTGCTGGCGCCAGACGACAGGAGCTACAAGTCCCAGTACCTCAACAACGGGCCGCAGCGCATTGGCAGGAAGTACAA |
| | GAAAGTCCGCTTCATGGCCTACACTGATGAAACCTTCAAGACGAGGGAAGCCATCCAGCACGAGTCAGGCATCCTGGG |
| | ACCGCTCCTTTACGGCGAAGTCGGGGATACCCTGCTCATCATTTTCAAGAACCAGGCATCGCGGCCCTACAACATCTA |
| | CCCTCACGGGATCACAGACGTGCGCCCGCTCTACTCCCGCCGGCTGCCCAAGGGAGTGAAGCACCTGAAGGATTTTCC |
| | CATCCTGCCGGGAGAAATCTTCAAGTACAAGTGGACCGTGACTGTGGAAGATGGCCCTACCAAGTCGGACCCTCGCTG |
| | TCTGACCCGGTACTATTCCTCGTTTGTGAACATGGAGCGCGACCTGGCCTCGGGGCTGATTGGTCCGCTGCTGATCTG |
| | CTACAAGGAGTCCGTGGACCAGCGCGGGAACCAGATCATGTCCGACAAGCGCAACGTGATCCTGTTCTCTGTCTTTGA |
| | TGAAAACAGATCGTGGTACTTGACTGAGAATATCCAGCGGTTCCTGCCCAACCCAGCGGGAGTGCAACTGGAGGACCC |
| | GGAGTTCCAGGCCTCAAACATTATGCACTCTATCAACGGCTATGTGTTCGACTCGCTCCAACTGAGCGTGTGCCTGCA |
| | TGAAGTGGCATACTGGTACATTCTGTCCATCGGAGCCCAGACCGACTTCCTGTCCGTGTTCTTCTCCGGATACACCTT |
| | CAAGCATAAGATGGTGTACGAGGACACTCTGACCCTCTTCCCATTTTCCGGAGAAACTGTGTTCATGTCAATGGAAAA |
| | CCCGGGCTTGTGGATTCTGGGTTGCCATAACTCGGACTTCCGGAATAGAGGGATGACCGCCCTGCTGAAAGTGTCCAG |
| | CTGTGACAAGAATACCGGCGATTACTACGAGGACAGCTATGAGGACATCTCCGCTTATCTGCTGTCCAAGAACAACGC |
| | CATTGAACCCAGGTCCTTCTCCCAAAACGGTGCACCGACCTCCGAAAGCGCCACCCCAGAGTCAGGACCTGGCTCGGA |
| | ACCGGCTACCTCGGGCTCAGAGACACCGGGGACTTCCGAGTCCGCAACCCCCGAGAGTGGACCCGGATCCGAACCAGC |
| | AACCTCAGGATCAGAAACCCCGGGAACTTCGGAATCCGCCACTCCCGAGTCGGGACCAGGCACCTCCACTGAGCCTTC |
| | CGAGGGAAGCGCCCCCGGATCCCCTGCTGGATCCCCTACCAGCACTGAAGAAGGCACCTCAGAATCCGCGACCCCTGA |
| | GTCCGGCCCTGGAAGCGAACCCGCCACCTCCGGTTCCGAAACCCCTGGGACTAGCGAGAGCGCCACTCCGGAATCGGG |
| | CCCAGGAAGCCCTGCCGGATCCCCGACCAGCACCGAGGAGGGAAGCCCCGCCGGGTCACCGACTTCCACTGAGGAGGG |
| | AGCCTCATCCCCCCCCGTGCTGAAGCGGCATCAAAGAGAGATCACCAGGACCACTCTCCAGTCCGATCAGGAAGAAAT |
| | TGACTACGACGATACTATCAGCGTGGAGATGAAGAAGGAGGACTTCGACATCTACGATGAGGATGAGAACCAGTCCCC |
| | TCGGAGCTTTCAGAAGAAAACCCGCCACTACTTCATCGCTGCCGTGGAGCGGCTGTGGGATTACGGGATGTCCAGCTC |
| | ACCGCATGTGCTGCGGAATAGAGCGCAGTCAGGATCGGTGCCCCAGTTCAAGAAGGTCGTGTTCCAAGAGTTCACCGA |
| | CGGGTCCTTCACTCAACCCCTGTACCGGGGCGAACTCAACGAACACCTGGGACTGCTTGGGCCGTATATCAGGGCAGA |
| | AGTGGAAGATAACATCATGGTCACCTTCCGCAACCAGGCCTCCCGGCCGTACAGCTTCTACTCTTCACTGATCTCCTA |
| | CGAGGAAGATCAGCGGCAGGGAGCCGAGCCCCGGAAGAACTTCGTCAAGCCTAACGAAACTAAGACCTACTTTTGGAA |
| | GGTCCAGCATCACATGGCCCCGACCAAAGACGAGTTCGACTGTAAAGCCTGGGCCTACTTCTCCGATGTGGACCTGGA |
| | GAAGGACGTGCACTCGGGACTCATTGGCCCGCTCCTTGTGTGCCATACTAATACCCTGAACCCTGCTCACGGTCGCCA |
| | AGTCACAGTGCAGGAGTTCGCCCTCTTCTTCACCATCTTCGATGAAACAAAGTCCTGGTACTTTACTGAGAACATGGA |
| | ACGCAATTGCAGGGCACCCTGCAACATCCAGATGGAAGATCCCACCTTCAAGGAAAACTACCGGTTTCATGCCATTAA |
| | CGGCTACATAATGGACACGTTGCCAGGACTGGTCATGGCCCAGGACCAGAGAATCCGGTGGTATCTGCTCTCCATGGG |
| | CTCCAACGAAAACATTCACAGCATTCATTTTTCCGGCCATGTGTTCACCGTCCGGAAGAAGGAAGAGTACAAGATGGC |
| | TCTGTACAACCTCTACCCTGGAGTGTTCGAGACTGTGGAAATGCTGCCTAGCAAGGCCGGCATTTGGAGAGTGGAATG |
| | CCTGATCGGAGAGCATTTGCACGCCGGAATGTCCACCCTGTTTCTTGTGTACTCCAACAAGTGCCAGACCCCGCTGGG |
| | AATGGCCTCAGGTCATATTAGGGATTTCCAGATCACTGCTTCGGGGCAGTACGGGCAGTGGGCACCTAAGTTGGCCCG |
| | GCTGCACTACTCTGGCTCCATCAATGCCTGGTCCACCAAGGAACCCTTCTCCTGGATTAAGGTGGACCTCCTGGCCCC |
| | AATGATTATTCACGGTATTAAGACCCAGGGTGCCCGACAGAAGTTCTCCTCACTCTACATCTCGCAATTCATCATAAT |
| | GTACAGCCTGGATGGGAAGAAGTGGCAGACCTACCGGGGAAACTCCACTGGAACGCTCATGGTGTTTTTCGGCAACGT |
| | GGACTCCTCCGGCATTAAGCACAACATCTTCAACCCTCCGATCATTGCTCGGTACATCCGGCTGCACCCAACTCACTA |
| | CAGCATCCGGTCCACCCTGCGGATGGAACTGATGGGTTGTGACCTGAACTCCTGCTCCATGCCCCTTGGGATGGAATC |
| | CAAGGCCATTAGCGATGCACAGATCACCGCCTCTTCATACTTCACCAACATGTTCGCGACCTGGTCCCCGTCGAAGGC |
| | CCGCCTGCACCTCCAAGGTCGCTCCAATGCGTGGCGGCCTCAAGTGAACAACCCCAAGGAGTGGCTCCAGGTCGACTT |
| | CCAAAAGACCATGAAGGTCACCGGAGTGACCACCCAGGGCGTGAAGTCCCTGCTGACCTCTATGTACGTTAAGGAGTT |
| | CCTCATCTCCTCAAGCCAAGACGGACATCAGTGGACCCTGTTCTTCCAAAACGGAAAAGTCAAAGTATTCCAGGGCAA |
| | CCAGGACTCCTTCACCCCTGTGGTCAACAGCCTGGACCCCCCATTGCTGACCCGCTACCTCCGCATCCACCCCCAAAG |
| | CTGGGTCCACCAGATCGCACTGCGCATGGAGGTCCTTGGATGCGAAGCCCAAGATCTGTACTAAGCGGCCGCTCATAA |
| | TCAACCTCTGGATTACAAAATTTGTGAAAGATTGACTGGTATTCTTAACTATGTTGCTCCTTTTACGCTATGTGGATA |
| | CGCTGCTTTAATGCCTTTGTATCATGCTATTGCTTCCCGTATGGCTTTCATTTTCTCCTCCTTGTATAAATCCTGGTT |
| | GCTGTCTCTTTATGAGGAGTTGTGGCCCGTTGTCAGGCAACGTGGCGTGGTGTGCACTGTGTTTGCTGACGCAACCCC |
| | CACTGGTTGGGGCATTGCCACCACCTGTCAGCTCCTTTCCGGGACTTTCGCTTTCCCCCTCCCTATTGCCACGGCGGA |
| | ACTCATCGCCGCCTGCCTTGCCCGCTGCTGGACAGGGGCTCGGCTGTTGGGCACTGACAATTCCGTGGTGTTGTCGGG |
| | GAAATCATCGTCCTTTCCTTGGCTGCTCGCCTGTGTTGCCACCTGGATTCTGCGCGGGACGTCCTTCTGCTACGTCCC |
| | TTCGGCCCTCAATCCAGCGGACCTTCCTTCCCGCGGCCTGCTGCCGGCTCTGCGGCCTCTTCCGCGTCTTCGCCTTCG |
| | CCCTCAGACGAGTCGGATCTCCCTTTGGGCCGCCTCCCCGCTGCCTAGGCGACTGTGCCTTCTAGTTGCCAGCCATCT |
| | GTTGTTTGCCCCTCCCCCGTGCCTTCCTTGACCCTGGAAGGTGCCACTCCCACTGTCCTTTCCTAATAAAATGAGGAA |
| | ATTGCATCGCATTGTCTGAGTAGGTGTCATTCTATTCTGGGGGGTGGGGTGGGGCAGGACAGCAAGGGGGAGGATTGG |
| | GAAGACAATAGCAGGCATGCTGGGGAAGACCATGGGCGCGCCAGGCCTGTCGACGCCCGGGCGGTACCGCGATCGCTC |
| | GCGACGCATAAAG |
| SEQ ID NO: 28 | ATGCAGATTGAACTGTCCACTTGCTTCTTCCTGTGCCTCCTGCGGTTTTGCTTCTCGGCCACCCGCCGGTATTACTTA |
| Nucleotide | GGTGCTGTGGAACTGAGCTGGGACTACATGCAGTCCGACCTGGGAGAACTGCCGGTGGACGCGAGATTCCCACCTAGA |
| sequence encoding | GTCCCGAAGTCCTTCCCATTCAACACCTCCGTGGTCTACAAAAAGACCCTGTTCGTGGAGTTCACTGACCACCTTTTC |
| coBDDFVIIIXTEN | AATATTGCCAAGCCGCGCCCCCCCTGGATGGGCCTGCTTGGTCCTACGATCCAAGCAGAGGTCTACGACACCGTGGTC |
| (V2.0) | ATCACACTGAAGAACATGGCCTCACACCCCGTGTCGCTGCATGCTGTGGGAGTGTCCTACTGGAAGGCCTCAGAGGGT |
| | GCCGAATATGATGACCAGACCAGCCAGAGGGAAAAGGAGGATGACAAAGTGTTCCCGGGTGGCAGCCACACTTACGTG |
| | TGGCAAGTGCTGAAGGAAAACGGGCCTATGGCGTCGGACCCCCTATGCCTGACCTACTCCTACCTGTCCCATGTGGAC |
| | CTTGTGAAGGATCTCAACTCGGGACTGATCGGCGCCCTCTTGGTGTGCAGAGAAGGCAGCCTGGCGAAGGAAAAGACT |
| | CAGACCCTGCACAAGTTCATTCTGTTGTTTGCTGTGTTCGATGAAGGAAAGTCCTGGCACTCAGAAACCAAGAACTCG |
| | CTGATGCAGGATAGAGATGCGGCCTCGGCCAGAGCCTGGCCTAAAATGCACACCGTCAACGGATATGTGAACAGGTCG |
| | CTCCCTGGCCTCATCGGCTGCCACAGAAAGTCCGTGTATTGGCATGTGATCGGCATGGGTACTACTCCGGAAGTGCAT |
| | AGTATCTTTCTGGAGGGCCATACCTTCTTGGTGCGCAACCACAGACAGGCCTCGCTGGAAATCTCGCCTATCACTTTC |
| | TTGACTGCGCAGACCCTCCTTATGGACCTTGGACAGTTCCTGCTGTTCTGTCACATCAGCTCCCATCAGCATGATGGG |
| | ATGGAGGCCTATGTCAAAGTGGACTCCTGCCCTGAGGAGCCACAGCTCCGGATGAAGAACAATGAGGAAGCGGAGGAT |
| | TACGACGACGACCTGACTGACAGCGAAATGGACGTCGTGCGATTCGATGACGACAACAGCCCGTCCTTCATCCAAATT |
| | AGATCAGTGGCGAAGAAGCACCCCAAGACCTGGGTGCACTACATTGCCGCCGAGGAAGAGGACTGGGACTACGCGCCG |
| | CTGGTGCTGGCGCCAGACGACAGGAGCTACAAGTCCCAGTACCTCAACAACGGGCCGCAGCGCATTGGCAGGAAGTAC |
| | AAGAAAGTCCGCTTCATGGCCTACACTGATGAAACCTTCAAGACGAGGGAAGCCATCCAGCACGAGTCAGGCATCCTG |
| | GGACCGCTCCTTTACGGCGAAGTCGGGGATACCCTGCTCATCATTTTCAAGAACCAGGCATCGCGGCCCTACAACATC |
| | TACCCTCACGGGATCACAGACGTGCGCCCGCTCTACTCCCGCCGGCTGCCCAAGGGAGTGAAGCACCTGAAGGATTTT |
| | CCCATCCTGCCGGGAGAAATCTTCAAGTACAAGTGGACCGTGACTGTGGAAGATGGCCCTACCAAGTCGGACCCTCGC |
| | TGTCTGACCCGGTACTATTCCTCGTTTGTGAACATGGAGCGCGACCTGGCCTCGGGGCTGATTGGTCCGCTGCTGATC |
| | TGCTACAAGGAGTCCGTGGACCAGCGCGGGAACCAGATCATGTCCGACAAGCGCAACGTGATCCTGTTCTCTGTCTTT |
| | GATGAAAACAGATCGTGGTACTTGACTGAGAATATCCAGCGGTTCCTGCCCAACCCAGCGGGAGTGCAACTGGAGGAC |
| | CCGGAGTTCCAGGCCTCAAACATTATGCACTCTATCAACGGCTATGTGTTCGACTCGCTCCAACTGAGCGTGTGCCTG |
| | CATGAAGTGGCATACTGGTACATTCTGTCCATCGGAGCCCAGACCGACTTCCTGTCCGTGTTCTTCTCCGGATACACC |
| | TTCAAGCATAAGATGGTGTACGAGGACACTCTGACCCTCTTCCCATTTTCCGGAGAAACTGTGTTCATGTCAATGGAA |
| | AACCCGGGCTTGTGGATTCTGGGTTGCCATAACTCGGACTTCCGGAATAGAGGGATGACCGCCCTGCTGAAAGTGTCC |
| | AGCTGTGACAAGAATACCGGCGATTACTACGAGGACAGCTATGAGGACATCTCCGCTTATCTGCTGTCCAAGAACAAC |
| | GCCATTGAACCCAGGTCCTTCTCCCAAAACGGTGCACCGACCTCCGAAAGCGCCACCCCAGAGTCAGGACCTGGCTCG |
| | GAACCGGCTACCTCGGGCTCAGAGACACCGGGGACTTCCGAGTCCGCAACCCCCGAGAGTGGACCCGGATCCGAACCA |
| | GCAACCTCAGGATCAGAAACCCCGGGAACTTCGGAATCCGCCACTCCCGAGTCGGGACCAGGCACCTCCACTGAGCCT |
| | TCCGAGGGAAGCGCCCCCGGATCCCCTGCTGGATCCCCTACCAGCACTGAAGAAGGCACCTCAGAATCCGCGACCCCT |
| | GAGTCCGGCCCTGGAAGCGAACCCGCCACCTCCGGTTCCGAAACCCCTGGGACTAGCGAGAGCGCCACTCCGGAATCG |
| | GGCCCAGGAAGCCCTGCCGGATCCCCGACCAGCACCGAGGAGGGAAGCCCCGCCGGGTCACCGACTTCCACTGAGGAG |
| | GGAGCCTCATCCCCCCCCGTGCTGAAGCGGCATCAAAGAGAGATCACCAGGACCACTCTCCAGTCCGATCAGGAAGAA |
| | ATTGACTACGACGATACTATCAGCGTGGAGATGAAGAAGGAGGACTTCGACATCTACGATGAGGATGAGAACCAGTCC |
| | CCTCGGAGCTTTCAGAAGAAAACCCGCCACTACTTCATCGCTGCCGTGGAGCGGCTGTGGGATTACGGGATGTCCAGC |
| | TCACCGCATGTGCTGCGGAATAGAGCGCAGTCAGGATCGGTGCCCCAGTTCAAGAAGGTCGTGTTCCAAGAGTTCACC |
| | GACGGGTCCTTCACTCAACCCCTGTACCGGGGCGAACTCAACGAACACCTGGGACTGCTTGGGCCGTATATCAGGGCA |
| | GAAGTGGAAGATAACATCATGGTCACCTTCCGCAACCAGGCCTCCCGGCCGTACAGCTTCTACTCTTCACTGATCTCC |
| | TACGAGGAAGATCAGCGGCAGGGAGCCGAGCCCCGGAAGAACTTCGTCAAGCCTAACGAAACTAAGACCTACTTTTGG |
| | AAGGTCCAGCATCACATGGCCCCGACCAAAGACGAGTTCGACTGTAAAGCCTGGGCCTACTTCTCCGATGTGGACCTG |
| | GAGAAGGACGTGCACTCGGGACTCATTGGCCCGCTCCTTGTGTGCCATACTAATACCCTGAACCCTGCTCACGGTCGC |
| | CAAGTCACAGTGCAGGAGTTCGCCCTCTTCTTCACCATCTTCGATGAAACAAAGTCCTGGTACTTTACTGAGAACATG |
| | GAACGCAATTGCAGGGCACCCTGCAACATCCAGATGGAAGATCCCACCTTCAAGGAAAACTACCGGTTTCATGCCATT |
| | AACGGCTACATAATGGACACGTTGCCAGGACTGGTCATGGCCCAGGACCAGAGAATCCGGTGGTATCTGCTCTCCATG |
| | GGCTCCAACGAAAACATTCACAGCATTCATTTTTCCGGCCATGTGTTCACCGTCCGGAAGAAGGAAGAGTACAAGATG |
| | GCTCTGTACAACCTCTACCCTGGAGTGTTCGAGACTGTGGAAATGCTGCCTAGCAAGGCCGGCATTTGGAGAGTGGAA |
| | TGCCTGATCGGAGAGCATTTGCACGCCGGAATGTCCACCCTGTTTCTTGTGTACTCCAACAAGTGCCAGACCCCGCTG |
| | GGAATGGCCTCAGGTCATATTAGGGATTTCCAGATCACTGCTTCGGGGCAGTACGGGCAGTGGGCACCTAAGTTGGCC |
| | CGGCTGCACTACTCTGGCTCCATCAATGCCTGGTCCACCAAGGAACCCTTCTCCTGGATTAAGGTGGACCTCCTGGCC |
| | CCAATGATTATTCACGGTATTAAGACCCAGGGTGCCCGACAGAAGTTCTCCTCACTCTACATCTCGCAATTCATCATA |
| | ATGTACAGCCTGGATGGGAAGAAGTGGCAGACCTACCGGGGAAACTCCACTGGAACGCTCATGGTGTTTTTCGGCAAC |
| | GTGGACTCCTCCGGCATTAAGCACAACATCTTCAACCCTCCGATCATTGCTCGGTACATCCGGCTGCACCCAACTCAC |
| | TACAGCATCCGGTCCACCCTGCGGATGGAACTGATGGGTTGTGACCTGAACTCCTGCTCCATGCCCCTTGGGATGGAA |
| | TCCAAGGCCATTAGCGATGCACAGATCACCGCCTCTTCATACTTCACCAACATGTTCGCGACCTGGTCCCCGTCGAAG |
| | GCCCGCCTGCACCTCCAAGGTCGCTCCAATGCGTGGCGGCCTCAAGTGAACAACCCCAAGGAGTGGCTCCAGGTCGAC |
| | TTCCAAAAGACCATGAAGGTCACCGGAGTGACCACCCAGGGCGTGAAGTCCCTGCTGACCTCTATGTACGTTAAGGAG |
| | TTCCTCATCTCCTCAAGCCAAGACGGACATCAGTGGACCCTGTTCTTCCAAAACGGAAAAGTCAAAGTATTCCAGGGC |
| | AACCAGGACTCCTTCACCCCTGTGGTCAACAGCCTGGACCCCCCATTGCTGACCCGCTACCTCCGCATCCACCCCCAA |
| | AGCTGGGTCCACCAGATCGCACTGCGCATGGAGGTCCTTGGATGCGAAGCCCAAGATCTGTACTAA |
| SEQ ID NO. 29: | GTATACCTGCAGGCTAGCCACGTGTTGTTGTTGTACATGCGCCATCTTAGTTTTATATCAGCTGGCGCCTTAGT |
| HBoV1-5′ITR- | TATATAACATGCATGTTATATAACTAAGGCGCCAGCTGATATAAAACTAAGATGGCGCATGTACAACAACAACA |
| mTTR482-Intron- | CATTAAAAGATATAGAGTTTCGCGATTGCAAGCTTGGCCCCAGGTTAATTTTTAAAAAGCAGTCAAAGGTCAAA |
| coBDDFVIIIXTEN | GTGGCCCTTGGCAGCATTTACTCTCTCTATTGACTTTGGTTAATAATCTCAGGAGCACAAACATTCCTGGAGGC |
| (V2.0)-WPRE- | AGGAGAAGAAATCAACATCCTGGACTTATCCTCTGGGCCTCTCCCCACCTTCGATGGCCCCAGGTTAATTTTTA |
| bGHPolyA- | AAAAGCAGTCAAAGGTCAAAGTGGCCCTTGGCAGCATTTACTCTCTCTATTGACTTTGGTTAATAATCTCAGGA |
| HBoV1-3′ITR | GCACAAACATTCCTGGAGGCAGGAGAAGAAATCAACATCCTGGACTTATCCTCTGGGCCTCTCCCCACCGATAT |
| | CTACCTGCTGATCGCCCGGCCCCTGTTCAAACATGTCCTAATACTCTGTCGGGGCAAAGGTCGGCAGTAGTTTT |
| | CCATCTTACTCAACATCCTCCCAGTGTACGTAGGATCCTGTCTGTCTGCACATTTCGTAGAGCGAGTGTTCCGA |
| | TACTCTAATCTCCCGGGGCAAAGGTCGTATTGACTTAGGTTACTTATTCTCCTTTTGTTGACTAAGTCAATAAT |
| | CAGAATCAGCAGGTTTGGAGTCAGCTTGGCAGGGATCAGCAGCCTGGGTTGGAAGGAGGGGGTATAAAAGCCCC |
| | TTCACCAGGAGAAGCCGTCACACAGATCCACAAGCTCCTGCTAGGAATTCTCAGGAGCACAAACATTCCTGGAG |
| | GCAGGAGAAGAAATCAACATCCTGGACTTATCCTCTGGGCCTCTCCCCACCGATATCTACCTGCTGATCGCCCG |
| | GCCCCTGTTCAAACATGTCCTAATACTCTGTCGGGGCAAAGGTCGGCAGTAGTTTTCCATCTTACTCAACATCC |
| | TCCCAGTGTACGTAGGATCCTGTCTGTCTGCACATTTCGTAGAGCGAGTGTTCCGATACTCTAATCTCCCGGGG |
| | CAAAGGTCGTATTGACTTAGGTTACTTATTCTCCTTTTGTTGACTAAGTCAATAATCAGAATCAGCAGGTTTGG |
| | AGTCAGCTTGGCAGGGATCAGCAGCCTGGGTTGGAAGGAGGGGGTATAAAAGCCCCTTCACCAGGAGAAGCCGT |
| | CACACAGATCCACAAGCTCCTGCTAGAGTCGCTGCGCGCTGCCTTCGCCCCGTGCCCCGCTCCGCCGCCGCCTC |
| | GCGCCGCCCGCCCCGGCTCTGACTGACCGCGTTACTCCCACAGGTGAGCGGGCGGGACGGCCCTTCTCCTCCGG |
| | GCTGTAATTAGCGCTTGGTTTATTGACGGCTTGTTTCTTTTCTGTGGCTGCGTGAAAGCCTTGAGGGGCTCCGG |
| | GAAGGCCCTTTGTGCGGGGGGAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGCGCTCCGCAGTGTGCGCGAGG |
| | GGAGCGCGGCCGGGGGCGGTGCCCCGCGGTGCGGGGGGGGCTGCGAGGGGAACAAAGGCTGCGTGCGGGGTGTG |
| | TGCGTGGGGGGGTGAGCAGGGGGTGTGGGCGCGTCGGTCGGGCTGCAACCCCCCCTGCACCCCCCTCCCCGAGT |
| | TGCTGAGCACGGCCCGGCTTCGGGTGCGGGGCTCCGTACGGGGCGTGGCGCGGGGCTCGCCGTGCCGGGCGGGG |
| | GGTGGCGGCAGGTGGGGGTGCCGGGCGGGGCGGGGCCGCCTCGGGCCGGGGAGGGCTCGGGGGAGGGGCGCGGC |
| | GGCCCCCGGAGCGCCGGCGGCTGTCGAGGCGCGGCGAGCCGCAGCCATTGCCTTTTATGGTAATCGTGCGAGAG |
| | GGCGCAGGGACTTCCTTTGTCCCAAATCTGTGCGGAGCCGAAATCTGGGAGGCGCCGCCGCACCCCCTCTAGCG |
| | GGCGCGGGGCGAAGCGGTGCGGCGCCGGCAGGAAGGAAATGGGCGGGGAGGGCCTTCGTGCGTCGCCGCGCCGC |
| | CGTCCCCTTCTCCCTCTCCAGCCTCGGGGCTGTCCGCGGGGGGACGGCTGCCTTCGGGGGGGACGGGGCAGGGC |
| | GGGGTTCGGCTTCTGGCGTGTGACCGGCGGCTCTAGAGCCTCTGCTAACCTTGTTCTTGCCTTCTTCTTTTTCC |
| | TACAGCTCCTGGGCAACGTGCTGGTTATTGTGCTGTCTCATCATTTTGGCAAAGAATTACTCGAGGCCACCATG |
| | CAGATTGAACTGTCCACTTGCTTCTTCCTGTGCCTCCTGCGGTTTTGCTTCTCGGCCACCCGCCGGTATTACTT |
| | AGGTGCTGTGGAACTGAGCTGGGACTACATGCAGTCCGACCTGGGAGAACTGCCGGTGGACGCGAGATTCCCAC |
| | CTAGAGTCCCGAAGTCCTTCCCATTCAACACCTCCGTGGTCTACAAAAAGACCCTGTTCGTGGAGTTCACTGAC |
| | CACCTTTTCAATATTGCCAAGCCGCGCCCCCCCTGGATGGGCCTGCTTGGTCCTACGATCCAAGCAGAGGTCTA |
| | CGACACCGTGGTCATCACACTGAAGAACATGGCCTCACACCCCGTGTCGCTGCATGCTGTGGGAGTGTCCTACT |
| | GGAAGGCCTCAGAGGGTGCCGAATATGATGACCAGACCAGCCAGAGGGAAAAGGAGGATGACAAAGTGTTCCCG |
| | GGTGGCAGCCACACTTACGTGTGGCAAGTGCTGAAGGAAAACGGGCCTATGGCGTCGGACCCCCTATGCCTGAC |
| | CTACTCCTACCTGTCCCATGTGGACCTTGTGAAGGATCTCAACTCGGGACTGATCGGCGCCCTCTTGGTGTGCA |
| | GAGAAGGCAGCCTGGCGAAGGAAAAGACTCAGACCCTGCACAAGTTCATTCTGTTGTTTGCTGTGTTCGATGAA |
| | GGAAAGTCCTGGCACTCAGAAACCAAGAACTCGCTGATGCAGGATAGAGATGCGGCCTCGGCCAGAGCCTGGCC |
| | TAAAATGCACACCGTCAACGGATATGTGAACAGGTCGCTCCCTGGCCTCATCGGCTGCCACAGAAAGTCCGTGT |
| | ATTGGCATGTGATCGGCATGGGTACTACTCCGGAAGTGCATAGTATCTTTCTGGAGGGCCATACCTTCTTGGTG |
| | CGCAACCACAGACAGGCCTCGCTGGAAATCTCGCCTATCACTTTCTTGACTGCGCAGACCCTCCTTATGGACCT |
| | TGGACAGTTCCTGCTGTTCTGTCACATCAGCTCCCATCAGCATGATGGGATGGAGGCCTATGTCAAAGTGGACT |
| | CCTGCCCTGAGGAGCCACAGCTCCGGATGAAGAACAATGAGGAAGCGGAGGATTACGACGACGACCTGACTGAC |
| | AGCGAAATGGACGTCGTGCGATTCGATGACGACAACAGCCCGTCCTTCATCCAAATTAGATCAGTGGCGAAGAA |
| | GCACCCCAAGACCTGGGTGCACTACATTGCCGCCGAGGAAGAGGACTGGGACTACGCGCCGCTGGTGCTGGCGC |
| | CAGACGACAGGAGCTACAAGTCCCAGTACCTCAACAACGGGCCGCAGCGCATTGGCAGGAAGTACAAGAAAGTC |
| | CGCTTCATGGCCTACACTGATGAAACCTTCAAGACGAGGGAAGCCATCCAGCACGAGTCAGGCATCCTGGGACC |
| | GCTCCTTTACGGCGAAGTCGGGGATACCCTGCTCATCATTTTCAAGAACCAGGCATCGCGGCCCTACAACATCT |
| | ACCCTCACGGGATCACAGACGTGCGCCCGCTCTACTCCCGCCGGCTGCCCAAGGGAGTGAAGCACCTGAAGGAT |
| | TTTCCCATCCTGCCGGGAGAAATCTTCAAGTACAAGTGGACCGTGACTGTGGAAGATGGCCCTACCAAGTCGGA |
| | CCCTCGCTGTCTGACCCGGTACTATTCCTCGTTTGTGAACATGGAGCGCGACCTGGCCTCGGGGCTGATTGGTC |
| | CGCTGCTGATCTGCTACAAGGAGTCCGTGGACCAGCGCGGGAACCAGATCATGTCCGACAAGCGCAACGTGATC |
| | CTGTTCTCTGTCTTTGATGAAAACAGATCGTGGTACTTGACTGAGAATATCCAGCGGTTCCTGCCCAACCCAGC |
| | GGGAGTGCAACTGGAGGACCCGGAGTTCCAGGCCTCAAACATTATGCACTCTATCAACGGCTATGTGTTCGACT |
| | CGCTCCAACTGAGCGTGTGCCTGCATGAAGTGGCATACTGGTACATTCTGTCCATCGGAGCCCAGACCGACTTC |
| | CTGTCCGTGTTCTTCTCCGGATACACCTTCAAGCATAAGATGGTGTACGAGGACACTCTGACCCTCTTCCCATT |
| | TTCCGGAGAAACTGTGTTCATGTCAATGGAAAACCCGGGCTTGTGGATTCTGGGTTGCCATAACTCGGACTTCC |
| | GGAATAGAGGGATGACCGCCCTGCTGAAAGTGTCCAGCTGTGACAAGAATACCGGCGATTACTACGAGGACAGC |
| | TATGAGGACATCTCCGCTTATCTGCTGTCCAAGAACAACGCCATTGAACCCAGGTCCTTCTCCCAAAACGGTGC |
| | ACCGACCTCCGAAAGCGCCACCCCAGAGTCAGGACCTGGCTCGGAACCGGCTACCTCGGGCTCAGAGACACCGG |
| | GGACTTCCGAGTCCGCAACCCCCGAGAGTGGACCCGGATCCGAACCAGCAACCTCAGGATCAGAAACCCCGGGA |
| | ACTTCGGAATCCGCCACTCCCGAGTCGGGACCAGGCACCTCCACTGAGCCTTCCGAGGGAAGCGCCCCCGGATC |
| | CCCTGCTGGATCCCCTACCAGCACTGAAGAAGGCACCTCAGAATCCGCGACCCCTGAGTCCGGCCCTGGAAGCG |
| | AACCCGCCACCTCCGGTTCCGAAACCCCTGGGACTAGCGAGAGCGCCACTCCGGAATCGGGCCCAGGAAGCCCT |
| | GCCGGATCCCCGACCAGCACCGAGGAGGGAAGCCCCGCCGGGTCACCGACTTCCACTGAGGAGGGAGCCTCATC |
| | CCCCCCCGTGCTGAAGCGGCATCAAAGAGAGATCACCAGGACCACTCTCCAGTCCGATCAGGAAGAAATTGACT |
| | ACGACGATACTATCAGCGTGGAGATGAAGAAGGAGGACTTCGACATCTACGATGAGGATGAGAACCAGTCCCCT |
| | CGGAGCTTTCAGAAGAAAACCCGCCACTACTTCATCGCTGCCGTGGAGCGGCTGTGGGATTACGGGATGTCCAG |
| | CTCACCGCATGTGCTGCGGAATAGAGCGCAGTCAGGATCGGTGCCCCAGTTCAAGAAGGTCGTGTTCCAAGAGT |
| | TCACCGACGGGTCCTTCACTCAACCCCTGTACCGGGGCGAACTCAACGAACACCTGGGACTGCTTGGGCCGTAT |
| | ATCAGGGCAGAAGTGGAAGATAACATCATGGTCACCTTCCGCAACCAGGCCTCCCGGCCGTACAGCTTCTACTC |
| | TTCACTGATCTCCTACGAGGAAGATCAGCGGCAGGGAGCCGAGCCCCGGAAGAACTTCGTCAAGCCTAACGAAA |
| | CTAAGACCTACTTTTGGAAGGTCCAGCATCACATGGCCCCGACCAAAGACGAGTTCGACTGTAAAGCCTGGGCC |
| | TACTTCTCCGATGTGGACCTGGAGAAGGACGTGCACTCGGGACTCATTGGCCCGCTCCTTGTGTGCCATACTAA |
| | TACCCTGAACCCTGCTCACGGTCGCCAAGTCACAGTGCAGGAGTTCGCCCTCTTCTTCACCATCTTCGATGAAA |
| | CAAAGTCCTGGTACTTTACTGAGAACATGGAACGCAATTGCAGGGCACCCTGCAACATCCAGATGGAAGATCCC |
| | ACCTTCAAGGAAAACTACCGGTTTCATGCCATTAACGGCTACATAATGGACACGTTGCCAGGACTGGTCATGGC |
| | CCAGGACCAGAGAATCCGGTGGTATCTGCTCTCCATGGGCTCCAACGAAAACATTCACAGCATTCATTTTTCCG |
| | GCCATGTGTTCACCGTCCGGAAGAAGGAAGAGTACAAGATGGCTCTGTACAACCTCTACCCTGGAGTGTTCGAG |
| | ACTGTGGAAATGCTGCCTAGCAAGGCCGGCATTTGGAGAGTGGAATGCCTGATCGGAGAGCATTTGCACGCCGG |
| | AATGTCCACCCTGTTTCTTGTGTACTCCAACAAGTGCCAGACCCCGCTGGGAATGGCCTCAGGTCATATTAGGG |
| | ATTTCCAGATCACTGCTTCGGGGCAGTACGGGCAGTGGGCACCTAAGTTGGCCCGGCTGCACTACTCTGGCTCC |
| | ATCAATGCCTGGTCCACCAAGGAACCCTTCTCCTGGATTAAGGTGGACCTCCTGGCCCCAATGATTATTCACGG |
| | TATTAAGACCCAGGGTGCCCGACAGAAGTTCTCCTCACTCTACATCTCGCAATTCATCATAATGTACAGCCTGG |
| | ATGGGAAGAAGTGGCAGACCTACCGGGGAAACTCCACTGGAACGCTCATGGTGTTTTTCGGCAACGTGGACTCC |
| | TCCGGCATTAAGCACAACATCTTCAACCCTCCGATCATTGCTCGGTACATCCGGCTGCACCCAACTCACTACAG |
| | CATCCGGTCCACCCTGCGGATGGAACTGATGGGTTGTGACCTGAACTCCTGCTCCATGCCCCTTGGGATGGAAT |
| | CCAAGGCCATTAGCGATGCACAGATCACCGCCTCTTCATACTTCACCAACATGTTCGCGACCTGGTCCCCGTCG |
| | AAGGCCCGCCTGCACCTCCAAGGTCGCTCCAATGCGTGGCGGCCTCAAGTGAACAACCCCAAGGAGTGGCTCCA |
| | GGTCGACTTCCAAAAGACCATGAAGGTCACCGGAGTGACCACCCAGGGCGTGAAGTCCCTGCTGACCTCTATGT |
| | ACGTTAAGGAGTTCCTCATCTCCTCAAGCCAAGACGGACATCAGTGGACCCTGTTCTTCCAAAACGGAAAAGTC |
| | AAAGTATTCCAGGGCAACCAGGACTCCTTCACCCCTGTGGTCAACAGCCTGGACCCCCCATTGCTGACCCGCTA |
| | CCTCCGCATCCACCCCCAAAGCTGGGTCCACCAGATCGCACTGCGCATGGAGGTCCTTGGATGCGAAGCCCAAG |
| | ATCTGTACTAAGCGGCCGCTCATAATCAACCTCTGGATTACAAAATTTGTGAAAGATTGACTGGTATTCTTAAC |
| | TATGTTGCTCCTTTTACGCTATGTGGATACGCTGCTTTAATGCCTTTGTATCATGCTATTGCTTCCCGTATGGC |
| | TTTCATTTTCTCCTCCTTGTATAAATCCTGGTTGCTGTCTCTTTATGAGGAGTTGTGGCCCGTTGTCAGGCAAC |
| | GTGGCGTGGTGTGCACTGTGTTTGCTGACGCAACCCCCACTGGTTGGGGCATTGCCACCACCTGTCAGCTCCTT |
| | TCCGGGACTTTCGCTTTCCCCCTCCCTATTGCCACGGCGGAACTCATCGCCGCCTGCCTTGCCCGCTGCTGGAC |
| | AGGGGCTCGGCTGTTGGGCACTGACAATTCCGTGGTGTTGTCGGGGAAATCATCGTCCTTTCCTTGGCTGCTCG |
| | CCTGTGTTGCCACCTGGATTCTGCGCGGGACGTCCTTCTGCTACGTCCCTTCGGCCCTCAATCCAGCGGACCTT |
| | CCTTCCCGCGGCCTGCTGCCGGCTCTGCGGCCTCTTCCGCGTCTTCGCCTTCGCCCTCAGACGAGTCGGATCTC |
| | CCTTTGGGCCGCCTCCCCGCTGCCTAGGCGACTGTGCCTTCTAGTTGCCAGCCATCTGTTGTTTGCCCCTCCCC |
| | CGTGCCTTCCTTGACCCTGGAAGGTGCCACTCCCACTGTCCTTTCCTAATAAAATGAGGAAATTGCATCGCATT |
| | GTCTGAGTAGGTGTCATTCTATTCTGGGGGGTGGGGTGGGGCAGGACAGCAAGGGGGAGGATTGGGAAGACAAT |
| | AGCAGGCATGCTGGGGAAGACCATGGGCGCGCCAGGCCTGTCGACGCCCGGGCGGTACCGCGATCGCTCGCGAC |
| | GCATAAAGTATATGTGACGTGGTTGTACAGACGCCATCTTGGAATCCAATATGTCTGCCGGCGATTAGATCATG |
| | CGCGCGCGCAGCGCGCTGCGCGCAGCGCAGGCATGACTGAGCCGGCAGACATATTGGATTCCAAGATGGCGTCT |
| | GTACAACCACGTGCTTAAGCTGCAGACTAGTGAGCTCGTTAAC |
| SEQ ID NO: 30 | GGCCCCAGGTTAATTTTTAAAAAGCAGTCAAAGGTCAAAGTGGCCCTTGGCAGCATTTACTCTCTCTATTGACT |
| A1MB2 enhancer | TTGGTTAATAATCTCAGGAGCACAAACATTCCTGGAGGCAGGAGAAGAAATCAACATCCTGGACTTATCCTCTG |
| | GGCCTCTCCCCACCTTCGATGGCCCCAGGTTAATTTTTAAAAAGCAGTCAAAGGTCAAAGTGGCCCTTGGCAGC |
| | ATTTACTCTCTCTATTGACTTTGGTTAATAATCTCAGGAGCACAAACATTCCTGGAGGCAGGAGAAGAAATCAA |
| | CATCCTGGACTTATCCTCTGGGCCTCTCCCCACC |
| SEQ ID NO: 31 | GATATCTACCTGCTGATCGCCCGGCCCCTGTTCAAACATGTCCTAATACTCTGTCGGGGCAAAGGTCGGCAGTA |
| mTTR promoter | GTTTTCCATCTTACTCAACATCCTCCCAGTGTACGTAGGATCCTGTCTGTCTGCACATTTCGTAGAGCGAGTGT |
| | TCCGATACTCTAATCTCCCGGGGCAAAGGTCGTATTGACTTAGGTTACTTATTCTCCTTTTGTTGACTAAGTCA |
| | ATAATCAGAATCAGCAGGTTTGGAGTCAGCTTGGCAGGGATCAGCAGCCTGGGTTGGAAGGAGGGGGTATAAAA |
| | GCCCCTTCACCAGGAGAAGCCGTCACACAGATCCACAAGCTCCTGCTAG |
| SEQ ID NO: 32 | TCAGGAGCACAAACATTCCTGGAGGCAGGAGAAGAAATCAACATCCTGGACTTATCCTCTGGGCCTCTCCCCAC |
| Chimeric Intron | CGATATCTACCTGCTGATCGCCCGGCCCCTGTTCAAACATGTCCTAATACTCTGTCGGGGCAAAGGTCGGCAGT |
| | AGTTTTCCATCTTACTCAACATCCTCCCAGTGTACGTAGGATCCTGTCTGTCTGCACATTTCGTAGAGCGAGTG |
| | TTCCGATACTCTAATCTCCCGGGGCAAAGGTCGTATTGACTTAGGTTACTTATTCTCCTTTTGTTGACTAAGTC |
| | AATAATCAGAATCAGCAGGTTTGGAGTCAGCTTGGCAGGGATCAGCAGCCTGGGTTGGAAGGAGGGGGTATAAA |
| | AGCCCCTTCACCAGGAGAAGCCGTCACACAGATCCACAAGCTCCTGCTAGAGTCGCTGCGCGCTGCCTTCGCCC |
| | CGTGCCCCGCTCCGCCGCCGCCTCGCGCCGCCCGCCCCGGCTCTGACTGACCGCGTTACTCCCACAGGTGAGCG |
| | GGCGGGACGGCCCTTCTCCTCCGGGCTGTAATTAGCGCTTGGTTTATTGACGGCTTGTTTCTTTTCTGTGGCTG |
| | CGTGAAAGCCTTGAGGGGCTCCGGGAAGGCCCTTTGTGCGGGGGGAGCGGCTCGGGGGGTGCGTGCGTGTGTGT |
| | GTGCGTGGGGAGCGCCGCGTGCGGCTCCGCGCTGCCCGGCGGCTGTGAGCGCTGCGGGCGCGGCGCGGGGCTTT |
| | GTGCGCTCCGCAGTGTGCGCGAGGGGAGCGCGGCCGGGGGCGGTGCCCCGCGGTGCGGGGGGGGCTGCGAGGGG |
| | AACAAAGGCTGCGTGCGGGGTGTGTGCGTGGGGGGGTGAGCAGGGGGTGTGGGCGCGTCGGTCGGGCTGCAACC |
| | CCCCCTGCACCCCCCTCCCCGAGTTGCTGAGCACGGCCCGGCTTCGGGTGCGGGGCTCCGTACGGGGCGTGGCG |
| | CGGGGCTCGCCGTGCCGGGCGGGGGGTGGCGGCAGGTGGGGGTGCCGGGCGGGGCGGGGCCGCCTCGGGCCGGG |
| | GAGGGCTCGGGGGAGGGGCGCGGCGGCCCCCGGAGCGCCGGCGGCTGTCGAGGCGCGGCGAGCCGCAGCCATTG |
| | CCTTTTATGGTAATCGTGCGAGAGGGCGCAGGGACTTCCTTTGTCCCAAATCTGTGCGGAGCCGAAATCTGGGA |
| | GGCGCCGCCGCACCCCCTCTAGCGGGCGCGGGGCGAAGCGGTGCGGCGCCGGCAGGAAGGAAATGGGCGGGGAG |
| | GGCCTTCGTGCGTCGCCGCGCCGCCGTCCCCTTCTCCCTCTCCAGCCTCGGGGCTGTCCGCGGGGGGACGGCTG |
| | CCTTCGGGGGGGACGGGGCAGGGCGGGGTTCGGCTTCTGGCGTGTGACCGGCGGCTCTAGAGCCTCTGCTAACC |
| | TTGTTCTTGCCTTCTTCTTTTTCCTACAGCTCCTGGGCAACGTGCTGGTTATTGTGCTGTCTCATCATTTTGGC |
| | AAAGAATTA |
| SEQ ID NO: 33 | TCATAATCAACCTCTGGATTACAAAATTTGTGAAAGATTGACTGGTATTCTTAACTATGTTGCTCCTTTTACGC |
| WPRE | TATGTGGATACGCTGCTTTAATGCCTTTGTATCATGCTATTGCTTCCCGTATGGCTTTCATTTTCTCCTCCTTG |
| | TATAAATCCTGGTTGCTGTCTCTTTATGAGGAGTTGTGGCCCGTTGTCAGGCAACGTGGCGTGGTGTGCACTGT |
| | GTTTGCTGACGCAACCCCCACTGGTTGGGGCATTGCCACCACCTGTCAGCTCCTTTCCGGGACTTTCGCTTTCC |
| | CCCTCCCTATTGCCACGGCGGAACTCATCGCCGCCTGCCTTGCCCGCTGCTGGACAGGGGCTCGGCTGTTGGGC |
| | ACTGACAATTCCGTGGTGTTGTCGGGGAAATCATCGTCCTTTCCTTGGCTGCTCGCCTGTGTTGCCACCTGGAT |
| | TCTGCGCGGGACGTCCTTCTGCTACGTCCCTTCGGCCCTCAATCCAGCGGACCTTCCTTCCCGCGGCCTGCTGC |
| | CGGCTCTGCGGCCTCTTCCGCGTCTTCGCCTTCGCCCTCAGACGAGTCGGATCTCCCTTTGGGCCGCCTCCCCG |
| | CTG |
| SEQ ID NO: 34 | CGACTGTGCCTTCTAGTTGCCAGCCATCTGTTGTTTGCCCCTCCCCCGTGCCTTCCTTGACCCTGGAAGGTGCC |
| bG HpA | ACTCCCACTGTCCTTTCCTAATAAAATGAGGAAATTGCATCGCATTGTCTGAGTAGGTGTCATTCTATTCTGGG |
| | GGGTGGGGTGGGGCAGGACAGCAAGGGGGAGGATTGGGAAGACAATAGCAGGCATGCTGGGGA |
| SEQ ID NO: 35 | GCCACTCGCCGGTACTACCTTGGAGCCGTGGAGCTTTCATGGGACTACATGCAGAGCGACCTGGGCGAACTCCC |
| Nucleotide | CGTGGATGCCAGATTCCCCCCCCGCGTGCCAAAGTCCTTCCCCTTTAACACCTCCGTGGTGTACAAGAAAACCC |
| sequence encoding | TCTTTGTCGAGTTCACTGACCACCTGTTCAACATCGCCAAGCCGCGCCCACCTTGGATGGGCCTCCTGGGACCG |
| BDD-co6FVIII | ACCATTCAAGCTGAAGTGTACGACACCGTGGTGATCACCCTGAAGAACATGGCGTCCCACCCCGTGTCCCTGCA |
| (V1.0) | TGCGGTCGGAGTGTCCTACTGGAAGGCCTCCGAAGGAGCTGAGTACGACGACCAGACTAGCCAGCGGGAAAAGG |
| (no XTEN) | AGGACGATAAAGTGTTCCCGGGCGGCTCGCATACTTACGTGTGGCAAGTCCTGAAGGAAAACGGACCTATGGCA |
| | TCCGATCCTCTGTGCCTGACTTACTCCTACCTTTCCCATGTGGACCTCGTGAAGGACCTGAACAGCGGGCTGAT |
| | TGGTGCACTTCTCGTGTGCCGCGAAGGTTCGCTCGCTAAGGAAAAGACCCAGACCCTCCATAAGTTCATCCTTT |
| | TGTTCGCTGTGTTCGATGAAGGAAAGTCATGGCATTCCGAAACTAAGAACTCGCTGATGCAGGACCGGGATGCC |
| | GCCTCAGCCCGCGCCTGGCCTAAAATGCATACAGTCAACGGATACGTGAATCGGTCACTGCCCGGGCTCATCGG |
| | TTGTCACAGAAAGTCCGTGTACTGGCACGTCATCGGCATGGGCACTACGCCTGAAGTGCACTCCATCTTCCTGG |
| | AAGGGCACACCTTCCTCGTGCGCAACCACCGCCAGGCCTCTCTGGAAATCTCCCCGATTACCTTTCTGACCGCC |
| | CAGACTCTGCTCATGGACCTGGGGCAGTTCCTTCTCTTCTGCCACATCTCCAGCCATCAGCACGACGGAATGGA |
| | GGCCTACGTGAAGGTGGACTCATGCCCGGAAGAACCTCAGTTGCGGATGAAGAACAACGAGGAGGCCGAGGACT |
| | ATGACGACGATTTGACTGACTCCGAGATGGACGTCGTGCGGTTCGATGACGACAACAGCCCCAGCTTCATCCAG |
| | ATTCGCAGCGTGGCCAAGAAGCACCCCAAAACCTGGGTGCACTACATCGCGGCCGAGGAAGAAGATTGGGACTA |
| | CGCCCCGTTGGTGCTGGCACCCGATGACCGGTCGTACAAGTCCCAGTATCTGAACAATGGTCCGCAGCGGATTG |
| | GCAGAAAGTACAAGAAAGTGCGGTTCATGGCGTACACTGACGAAACGTTTAAGACCCGGGAGGCCATTCAACAT |
| | GAGAGCGGCATTCTGGGACCACTGCTGTACGGAGAGGTCGGCGATACCCTGCTCATCATCTTCAAAAACCAGGC |
| | CTCCCGGCCTTACAACATCTACCCTCACGGAATCACCGACGTGCGGCCACTCTACTCGCGGCGCCTGCCGAAGG |
| | GCGTCAAGCACCTGAAAGACTTCCCTATCCTGCCGGGCGAAATCTTCAAGTATAAGTGGACCGTCACCGTGGAG |
| | GACGGGCCCACCAAGAGCGATCCTAGGTGTCTGACTCGGTACTACTCCAGCTTCGTGAACATGGAACGGGACCT |
| | GGCATCGGGACTCATTGGACCGCTGCTGATCTGCTACAAAGAGTCGGTGGATCAACGCGGCAACCAGATCATGT |
| | CCGACAAGCGCAACGTGATCCTGTTCTCCGTGTTTGATGAAAACAGATCCTGGTACCTCACTGAAAACATCCAG |
| | AGGTTCCTCCCAAACCCCGCAGGAGTGCAACTGGAGGACCCTGAGTTTCAGGCCTCGAATATCATGCACTCGAT |
| | TAACGGTTACGTGTTCGACTCGCTGCAGCTGAGCGTGTGCCTCCATGAAGTCGCTTACTGGTACATTCTGTCCA |
| | TCGGCGCCCAGACTGACTTCCTGAGCGTGTTCTTTTCCGGTTACACCTTTAAGCACAAGATGGTGTACGAAGAT |
| | ACCCTGACCCTGTTCCCTTTCTCCGGCGAAACGGTGTTCATGTCGATGGAGAACCCGGGTCTGTGGATTCTGGG |
| | ATGCCACAACAGCGACTTTCGGAACCGCGGAATGACTGCCCTGCTGAAGGTGTCCTCATGCGACAAGAACACCG |
| | GAGACTACTACGAGGACTCCTACGAGGATATCTCAGCCTACCTCCTGTCCAAGAACAACGCGATCGAGCCGCGC |
| | AGCTTCAGCCAGAACCCGCCTGTGCTGAAGAGGCACCAGCGAGAAATTACCCGGACCACCCTCCAATCGGATCA |
| | GGAGGAAATCGACTACGACGACACCATCTCGGTGGAAATGAAGAAGGAAGATTTCGATATCTACGACGAGGACG |
| | AAAATCAGTCCCCTCGCTCATTCCAAAAGAAAACTAGACACTACTTTATCGCCGCGGTGGAAAGACTGTGGGAC |
| | TATGGAATGTCATCCAGCCCTCACGTCCTTCGGAACCGGGCCCAGAGCGGATCGGTGCCTCAGTTCAAGAAAGT |
| | GGTGTTCCAGGAGTTCACCGACGGCAGCTTCACCCAGCCGCTGTACCGGGGAGAACTGAACGAACACCTGGGCC |
| | TGCTCGGTCCCTACATCCGCGCGGAAGTGGAGGATAACATCATGGTGACCTTCCGTAACCAAGCATCCAGACCT |
| | TACTCCTTCTATTCCTCCCTGATCTCATACGAGGAGGACCAGCGCCAAGGCGCCGAGCCCCGCAAGAACTTCGT |
| | CAAGCCCAACGAGACTAAGACCTACTTCTGGAAGGTCCAACACCATATGGCCCCGACCAAGGATGAGTTTGACT |
| | GCAAGGCCTGGGCCTACTTCTCCGACGTGGACCTTGAGAAGGATGTCCATTCCGGCCTGATCGGGCCGCTGCTC |
| | GTGTGTCACACCAACACCCTGAACCCAGCGCATGGACGCCAGGTCACCGTCCAGGAGTTTGCTCTGTTCTTCAC |
| | CATTTTTGACGAAACTAAGTCCTGGTACTTCACCGAGAATATGGAGCGAAACTGTAGAGCGCCCTGCAATATCC |
| | AGATGGAAGATCCGACTTTCAAGGAGAACTATAGATTCCACGCCATCAACGGGTACATCATGGATACTCTGCCG |
| | GGGCTGGTCATGGCCCAGGATCAGAGGATTCGGTGGTACTTGCTGTCAATGGGATCGAACGAAAACATTCACTC |
| | CATTCACTTCTCCGGTCACGTGTTCACTGTGCGCAAGAAGGAGGAGTACAAGATGGCGCTGTACAATCTGTACC |
| | CCGGGGTGTTCGAAACTGTGGAGATGCTGCCGTCCAAGGCCGGCATCTGGAGAGTGGAGTGCCTGATCGGAGAG |
| | CACCTCCACGCGGGGATGTCCACCCTCTTCCTGGTGTACTCGAATAAGTGCCAGACCCCGCTGGGCATGGCCTC |
| | GGGCCACATCAGAGACTTCCAGATCACAGCAAGCGGACAATACGGCCAATGGGCGCCGAAGCTGGCCCGCTTGC |
| | ACTACTCCGGATCGATCAACGCATGGTCCACCAAGGAACCGTTCTCGTGGATTAAGGTGGACCTCCTGGCCCCT |
| | ATGATTATCCACGGAATTAAGACCCAGGGCGCCAGGCAGAAGTTCTCCTCCCTGTACATCTCGCAATTCATCAT |
| | CATGTACAGCCTGGACGGGAAGAAGTGGCAGACTTACAGGGGAAACTCCACCGGCACCCTGATGGTCTTTTTCG |
| | GCAACGTGGATTCCTCCGGCATTAAGCACAACATCTTCAACCCACCGATCATAGCCAGATATATTAGGCTCCAC |
| | CCCACTCACTACTCAATCCGCTCAACTCTTCGGATGGAACTCATGGGGTGCGACCTGAACTCCTGCTCCATGCC |
| | GTTGGGGATGGAATCAAAGGCTATTAGCGACGCCCAGATCACCGCGAGCTCCTACTTCACTAACATGTTCGCCA |
| | CCTGGAGCCCCTCCAAGGCCAGGCTGCACTTGCAGGGACGGTCAAATGCCTGGCGGCCGCAAGTGAACAATCCG |
| | AAGGAATGGCTTCAAGTGGATTTCCAAAAGACCATGAAAGTGACCGGAGTCACCACCCAGGGAGTGAAGTCCCT |
| | TCTGACCTCGATGTATGTGAAGGAGTTCCTGATTAGCAGCAGCCAGGACGGGCACCAGTGGACCCTGTTCTTCC |
| | AAAACGGAAAGGTCAAGGTGTTCCAGGGGAACCAGGACTCGTTCACACCCGTGGTGAACTCCCTGGACCCCCCA |
| | CTGCTGACGCGGTACTTGAGGATTCATCCTCAGTCCTGGGTCCATCAGATTGCATTGCGAATGGAAGTCCTGGG |
| | CTGCGAGGCCCAGGACCTGTACTGA |
| SEQ ID NO: 36 | GCCACCCGCCGGTATTACTTAGGTGCTGTGGAACTGAGCTGGGACTACATGCAGTCCGACCTGGGAGAACTGCC |
| Nucleotide | GGTGGACGCGAGATTCCCACCTAGAGTCCCGAAGTCCTTCCCATTCAACACCTCCGTGGTCTACAAAAAGACCC |
| sequence encoding | TGTTCGTGGAGTTCACTGACCACCTTTTCAATATTGCCAAGCCGCGCCCCCCCTGGATGGGCCTGCTTGGTCCT |
| coBDDFVIII | ACGATCCAAGCAGAGGTCTACGACACCGTGGTCATCACACTGAAGAACATGGCCTCACACCCCGTGTCGCTGCA |
| (V2.0) | TGCTGTGGGAGTGTCCTACTGGAAGGCCTCAGAGGGTGCCGAATATGATGACCAGACCAGCCAGAGGGAAAAGG |
| (no XTEN) | AGGATGACAAAGTGTTCCCGGGTGGCAGCCACACTTACGTGTGGCAAGTGCTGAAGGAAAACGGGCCTATGGCG |
| | TCGGACCCCCTATGCCTGACCTACTCCTACCTGTCCCATGTGGACCTTGTGAAGGATCTCAACTCGGGACTGAT |
| | CGGCGCCCTCTTGGTGTGCAGAGAAGGCAGCCTGGCGAAGGAAAAGACTCAGACCCTGCACAAGTTCATTCTGT |
| | TGTTTGCTGTGTTCGATGAAGGAAAGTCCTGGCACTCAGAAACCAAGAACTCGCTGATGCAGGATAGAGATGCG |
| | GCCTCGGCCAGAGCCTGGCCTAAAATGCACACCGTCAACGGATATGTGAACAGGTCGCTCCCTGGCCTCATCGG |
| | CTGCCACAGAAAGTCCGTGTATTGGCATGTGATCGGCATGGGTACTACTCCGGAAGTGCATAGTATCTTTCTGG |
| | AGGGCCATACCTTCTTGGTGCGCAACCACAGACAGGCCTCGCTGGAAATCTCGCCTATCACTTTCTTGACTGCG |
| | CAGACCCTCCTTATGGACCTTGGACAGTTCCTGCTGTTCTGTCACATCAGCTCCCATCAGCATGATGGGATGGA |
| | GGCCTATGTCAAAGTGGACTCCTGCCCTGAGGAGCCACAGCTCCGGATGAAGAACAATGAGGAAGCGGAGGATT |
| | ACGACGACGACCTGACTGACAGCGAAATGGACGTCGTGCGATTCGATGACGACAACAGCCCGTCCTTCATCCAA |
| | ATTAGATCAGTGGCGAAGAAGCACCCCAAGACCTGGGTGCACTACATTGCCGCCGAGGAAGAGGACTGGGACTA |
| | CGCGCCGCTGGTGCTGGCGCCAGACGACAGGAGCTACAAGTCCCAGTACCTCAACAACGGGCCGCAGCGCATTG |
| | GCAGGAAGTACAAGAAAGTCCGCTTCATGGCCTACACTGATGAAACCTTCAAGACGAGGGAAGCCATCCAGCAC |
| | GAGTCAGGCATCCTGGGACCGCTCCTTTACGGCGAAGTCGGGGATACCCTGCTCATCATTTTCAAGAACCAGGC |
| | ATCGCGGCCCTACAACATCTACCCTCACGGGATCACAGACGTGCGCCCGCTCTACTCCCGCCGGCTGCCCAAGG |
| | GAGTGAAGCACCTGAAGGATTTTCCCATCCTGCCGGGAGAAATCTTCAAGTACAAGTGGACCGTGACTGTGGAA |
| | GATGGCCCTACCAAGTCGGACCCTCGCTGTCTGACCCGGTACTATTCCTCGTTTGTGAACATGGAGCGCGACCT |
| | GGCCTCGGGGCTGATTGGTCCGCTGCTGATCTGCTACAAGGAGTCCGTGGACCAGCGCGGGAACCAGATCATGT |
| | CCGACAAGCGCAACGTGATCCTGTTCTCTGTCTTTGATGAAAACAGATCGTGGTACTTGACTGAGAATATCCAG |
| | CGGTTCCTGCCCAACCCAGCGGGAGTGCAACTGGAGGACCCGGAGTTCCAGGCCTCAAACATTATGCACTCTAT |
| | CAACGGCTATGTGTTCGACTCGCTCCAACTGAGCGTGTGCCTGCATGAAGTGGCATACTGGTACATTCTGTCCA |
| | TCGGAGCCCAGACCGACTTCCTGTCCGTGTTCTTCTCCGGATACACCTTCAAGCATAAGATGGTGTACGAGGAC |
| | ACTCTGACCCTCTTCCCATTTTCCGGAGAAACTGTGTTCATGTCAATGGAAAACCCGGGCTTGTGGATTCTGGG |
| | TTGCCATAACTCGGACTTCCGGAATAGAGGGATGACCGCCCTGCTGAAAGTGTCCAGCTGTGACAAGAATACCG |
| | GCGATTACTACGAGGACAGCTATGAGGACATCTCCGCTTATCTGCTGTCCAAGAACAACGCCATTGAACCCAGG |
| | TCCTTCTCCCAAAACGGTGCACCGGCCTCATCCCCCCCCGTGCTGAAGCGGCATCAAAGAGAGATCACCAGGAC |
| | CACTCTCCAGTCCGATCAGGAAGAAATTGACTACGACGATACTATCAGCGTGGAGATGAAGAAGGAGGACTTCG |
| | ACATCTACGATGAGGATGAGAACCAGTCCCCTCGGAGCTTTCAGAAGAAAACCCGCCACTACTTCATCGCTGCC |
| | GTGGAGCGGCTGTGGGATTACGGGATGTCCAGCTCACCGCATGTGCTGCGGAATAGAGCGCAGTCAGGATCGGT |
| | GCCCCAGTTCAAGAAGGTCGTGTTCCAAGAGTTCACCGACGGGTCCTTCACTCAACCCCTGTACCGGGGCGAAC |
| | TCAACGAACACCTGGGACTGCTTGGGCCGTATATCAGGGCAGAAGTGGAAGATAACATCATGGTCACCTTCCGC |
| | AACCAGGCCTCCCGGCCGTACAGCTTCTACTCTTCACTGATCTCCTACGAGGAAGATCAGCGGCAGGGAGCCGA |
| | GCCCCGGAAGAACTTCGTCAAGCCTAACGAAACTAAGACCTACTTTTGGAAGGTCCAGCATCACATGGCCCCGA |
| | CCAAAGACGAGTTCGACTGTAAAGCCTGGGCCTACTTCTCCGATGTGGACCTGGAGAAGGACGTGCACTCGGGA |
| | CTCATTGGCCCGCTCCTTGTGTGCCATACTAATACCCTGAACCCTGCTCACGGTCGCCAAGTCACAGTGCAGGA |
| | GTTCGCCCTCTTCTTCACCATCTTCGATGAAACAAAGTCCTGGTACTTTACTGAGAACATGGAACGCAATTGCA |
| | GGGCACCCTGCAACATCCAGATGGAAGATCCCACCTTCAAGGAAAACTACCGGTTTCATGCCATTAACGGCTAC |
| | ATAATGGACACGTTGCCAGGACTGGTCATGGCCCAGGACCAGAGAATCCGGTGGTATCTGCTCTCCATGGGCTC |
| | CAACGAAAACATTCACAGCATTCATTTTTCCGGCCATGTGTTCACCGTCCGGAAGAAGGAAGAGTACAAGATGG |
| | CTCTGTACAACCTCTACCCTGGAGTGTTCGAGACTGTGGAAATGCTGCCTAGCAAGGCCGGCATTTGGAGAGTG |
| | GAATGCCTGATCGGAGAGCATTTGCACGCCGGAATGTCCACCCTGTTTCTTGTGTACTCCAACAAGTGCCAGAC |
| | CCCGCTGGGAATGGCCTCAGGTCATATTAGGGATTTCCAGATCACTGCTTCGGGGCAGTACGGGCAGTGGGCAC |
| | CTAAGTTGGCCCGGCTGCACTACTCTGGCTCCATCAATGCCTGGTCCACCAAGGAACCCTTCTCCTGGATTAAG |
| | GTGGACCTCCTGGCCCCAATGATTATTCACGGTATTAAGACCCAGGGTGCCCGACAGAAGTTCTCCTCACTCTA |
| | CATCTCGCAATTCATCATAATGTACAGCCTGGATGGGAAGAAGTGGCAGACCTACCGGGGAAACTCCACTGGAA |
| | CGCTCATGGTGTTTTTCGGCAACGTGGACTCCTCCGGCATTAAGCACAACATCTTCAACCCTCCGATCATTGCT |
| | CGGTACATCCGGCTGCACCCAACTCACTACAGCATCCGGTCCACCCTGCGGATGGAACTGATGGGTTGTGACCT |
| | GAACTCCTGCTCCATGCCCCTTGGGATGGAATCCAAGGCCATTAGCGATGCACAGATCACCGCCTCTTCATACT |
| | TCACCAACATGTTCGCGACCTGGTCCCCGTCGAAGGCCCGCCTGCACCTCCAAGGTCGCTCCAATGCGTGGCGG |
| | CCTCAAGTGAACAACCCCAAGGAGTGGCTCCAGGTCGACTTCCAAAAGACCATGAAGGTCACCGGAGTGACCAC |
| | CCAGGGCGTGAAGTCCCTGCTGACCTCTATGTACGTTAAGGAGTTCCTCATCTCCTCAAGCCAAGACGGACATC |
| | AGTGGACCCTGTTCTTCCAAAACGGAAAAGTCAAAGTATTCCAGGGCAACCAGGACTCCTTCACCCCTGTGGTC |
| | AACAGCCTGGACCCCCCATTGCTGACCCGCTACCTCCGCATCCACCCCCAAAGCTGGGTCCACCAGATCGCACT |
| | GCGCATGGAGGTCCTTGGATGCGAAGCCCAAGATCTGTACTAA |
| SEQ ID NO: 37 | ATGCAGATTGAGCTGTCCACTTGTTTCTTCCTGTGCCTCCTGCGCTTCTGTTTCTCCGCCACTCGCCGGTACTA |
| V1.0 | CCTTGGAGCCGTGGAGCTTTCATGGGACTACATGCAGAGCGACCTGGGCGAACTCCCCGTGGATGCCAGATTCC |
| Expression | CCCCCCGCGTGCCAAAGTCCTTCCCCTTTAACACCTCCGTGGTGTACAAGAAAACCCTCTTTGTCGAGTTCACT |
| cassette | GACCACCTGTTCAACATCGCCAAGCCGCGCCCACCTTGGATGGGCCTCCTGGGACCGACCATTCAAGCTGAAGT |
| TTP-Intron- | GTACGACACCGTGGTGATCACCCTGAAGAACATGGCGTCCCACCCCGTGTCCCTGCATGCGGTCGGAGTGTCCT |
| BDDFVIIIco6XTEN | ACTGGAAGGCCTCCGAAGGAGCTGAGTACGACGACCAGACTAGCCAGCGGGAAAAGGAGGACGATAAAGTGTTC |
| (V1.0)-WPRE- | CCGGGCGGCTCGCATACTTACGTGTGGCAAGTCCTGAAGGAAAACGGACCTATGGCATCCGATCCTCTGTGCCT |
| bG HPolyA | GACTTACTCCTACCTTTCCCATGTGGACCTCGTGAAGGACCTGAACAGCGGGCTGATTGGTGCACTTCTCGTGT |
| expression | GCCGCGAAGGTTCGCTCGCTAAGGAAAAGACCCAGACCCTCCATAAGTTCATCCTTTTGTTCGCTGTGTTCGAT |
| cassette | GAAGGAAAGTCATGGCATTCCGAAACTAAGAACTCGCTGATGCAGGACCGGGATGCCGCCTCAGCCCGCGCCTG |
| | GCCTAAAATGCATACAGTCAACGGATACGTGAATCGGTCACTGCCCGGGCTCATCGGTTGTCACAGAAAGTCCG |
| | TGTACTGGCACGTCATCGGCATGGGCACTACGCCTGAAGTGCACTCCATCTTCCTGGAAGGGCACACCTTCCTC |
| | GTGCGCAACCACCGCCAGGCCTCTCTGGAAATCTCCCCGATTACCTTTCTGACCGCCCAGACTCTGCTCATGGA |
| | CCTGGGGCAGTTCCTTCTCTTCTGCCACATCTCCAGCCATCAGCACGACGGAATGGAGGCCTACGTGAAGGTGG |
| | ACTCATGCCCGGAAGAACCTCAGTTGCGGATGAAGAACAACGAGGAGGCCGAGGACTATGACGACGATTTGACT |
| | GACTCCGAGATGGACGTCGTGCGGTTCGATGACGACAACAGCCCCAGCTTCATCCAGATTCGCAGCGTGGCCAA |
| | GAAGCACCCCAAAACCTGGGTGCACTACATCGCGGCCGAGGAAGAAGATTGGGACTACGCCCCGTTGGTGCTGG |
| | CACCCGATGACCGGTCGTACAAGTCCCAGTATCTGAACAATGGTCCGCAGCGGATTGGCAGAAAGTACAAGAAA |
| | GTGCGGTTCATGGCGTACACTGACGAAACGTTTAAGACCCGGGAGGCCATTCAACATGAGAGCGGCATTCTGGG |
| | ACCACTGCTGTACGGAGAGGTCGGCGATACCCTGCTCATCATCTTCAAAAACCAGGCCTCCCGGCCTTACAACA |
| | TCTACCCTCACGGAATCACCGACGTGCGGCCACTCTACTCGCGGCGCCTGCCGAAGGGCGTCAAGCACCTGAAA |
| | GACTTCCCTATCCTGCCGGGCGAAATCTTCAAGTATAAGTGGACCGTCACCGTGGAGGACGGGCCCACCAAGAG |
| | CGATCCTAGGTGTCTGACTCGGTACTACTCCAGCTTCGTGAACATGGAACGGGACCTGGCATCGGGACTCATTG |
| | GACCGCTGCTGATCTGCTACAAAGAGTCGGTGGATCAACGCGGCAACCAGATCATGTCCGACAAGCGCAACGTG |
| | ATCCTGTTCTCCGTGTTTGATGAAAACAGATCCTGGTACCTCACTGAAAACATCCAGAGGTTCCTCCCAAACCC |
| | CGCAGGAGTGCAACTGGAGGACCCTGAGTTTCAGGCCTCGAATATCATGCACTCGATTAACGGTTACGTGTTCG |
| | ACTCGCTGCAACTGAGCGTGTGCCTCCATGAAGTCGCTTACTGGTACATTCTGTCCATCGGCGCCCAGACTGAC |
| | TTCCTGAGCGTGTTCTTTTCCGGTTACACCTTTAAGCACAAGATGGTGTACGAAGATACCCTGACCCTGTTCCC |
| | TTTCTCCGGCGAAACGGTGTTCATGTCGATGGAGAACCCGGGTCTGTGGATTCTGGGATGCCACAACAGCGACT |
| | TTCGGAACCGCGGAATGACTGCCCTGCTGAAGGTGTCCTCATGCGACAAGAACACCGGAGACTACTACGAGGAC |
| | TCCTACGAGGATATCTCAGCCTACCTCCTGTCCAAGAACAACGCGATCGAGCCGCGCAGCTTCAGCCAGAACGG |
| | CGCGCCAACATCAGAGAGCGCCACCCCTGAAAGTGGTCCCGGGAGCGAGCCAGCCACATCTGGGTCGGAAACGC |
| | CAGGCACAAGTGAGTCTGCAACTCCCGAGTCCGGACCTGGCTCCGAGCCTGCCACTAGCGGCTCCGAGACTCCG |
| | GGAACTTCCGAGAGCGCTACACCAGAAAGCGGACCCGGAACCAGTACCGAACCTAGCGAGGGCTCTGCTCCGGG |
| | CAGCCCAGCCGGCTCTCCTACATCCACGGAGGAGGGCACTTCCGAATCCGCCACCCCGGAGTCAGGGCCAGGAT |
| | CTGAACCCGCTACCTCAGGCAGTGAGACGCCAGGAACGAGCGAGTCCGCTACACCGGAGAGTGGGCCAGGGAGC |
| | CCTGCTGGATCTCCTACGTCCACTGAGGAAGGGTCACCAGCGGGCTCGCCCACCAGCACTGAAGAAGGTGCCTC |
| | GAGCCCGCCTGTGCTGAAGAGGCACCAGCGAGAAATTACCCGGACCACCCTCCAATCGGATCAGGAGGAAATCG |
| | ACTACGACGACACCATCTCGGTGGAAATGAAGAAGGAAGATTTCGATATCTACGACGAGGACGAAAATCAGTCC |
| | CCTCGCTCATTCCAAAAGAAAACTAGACACTACTTTATCGCCGCGGTGGAAAGACTGTGGGACTATGGAATGTC |
| | ATCCAGCCCTCACGTCCTTCGGAACCGGGCCCAGAGCGGATCGGTGCCTCAGTTCAAGAAAGTGGTGTTCCAGG |
| | AGTTCACCGACGGCAGCTTCACCCAGCCGCTGTACCGGGGAGAACTGAACGAACACCTGGGCCTGCTCGGTCCC |
| | TACATCCGCGCGGAAGTGGAGGATAACATCATGGTGACCTTCCGTAACCAAGCATCCAGACCTTACTCCTTCTA |
| | TTCCTCCCTGATCTCATACGAGGAGGACCAGCGCCAAGGCGCCGAGCCCCGCAAGAACTTCGTCAAGCCCAACG |
| | AGACTAAGACCTACTTCTGGAAGGTCCAACACCATATGGCCCCGACCAAGGATGAGTTTGACTGCAAGGCCTGG |
| | GCCTACTTCTCCGACGTGGACCTTGAGAAGGATGTCCATTCCGGCCTGATCGGGCCGCTGCTCGTGTGTCACAC |
| | CAACACCCTGAACCCAGCGCATGGACGCCAGGTCACCGTCCAGGAGTTTGCTCTGTTCTTCACCATTTTTGACG |
| | AAACTAAGTCCTGGTACTTCACCGAGAATATGGAGCGAAACTGTAGAGCGCCCTGCAATATCCAGATGGAAGAT |
| | CCGACTTTCAAGGAGAACTATAGATTCCACGCCATCAACGGGTACATCATGGATACTCTGCCGGGGCTGGTCAT |
| | GGCCCAGGATCAGAGGATTCGGTGGTACTTGCTGTCAATGGGATCGAACGAAAACATTCACTCCATTCACTTCT |
| | CCGGTCACGTGTTCACTGTGCGCAAGAAGGAGGAGTACAAGATGGCGCTGTACAATCTGTACCCCGGGGTGTTC |
| | GAAACTGTGGAGATGCTGCCGTCCAAGGCCGGCATCTGGAGAGTGGAGTGCCTGATCGGAGAGCACCTCCACGC |
| | GGGGATGTCCACCCTCTTCCTGGTGTACTCGAATAAGTGCCAGACCCCGCTGGGCATGGCCTCGGGCCACATCA |
| | GAGACTTCCAGATCACAGCAAGCGGACAATACGGCCAATGGGCGCCGAAGCTGGCCCGCTTGCACTACTCCGGA |
| | TCGATCAACGCATGGTCCACCAAGGAACCGTTCTCGTGGATTAAGGTGGACCTCCTGGCCCCTATGATTATCCA |
| | CGGAATTAAGACCCAGGGCGCCAGGCAGAAGTTCTCCTCCCTGTACATCTCGCAATTCATCATCATGTACAGCC |
| | TGGACGGGAAGAAGTGGCAGACTTACAGGGGAAACTCCACCGGCACCCTGATGGTCTTTTTCGGCAACGTGGAT |
| | TCCTCCGGCATTAAGCACAACATCTTCAACCCACCGATCATAGCCAGATATATTAGGCTCCACCCCACTCACTA |
| | CTCAATCCGCTCAACTCTTCGGATGGAACTCATGGGGTGCGACCTGAACTCCTGCTCCATGCCGTTGGGGATGG |
| | AATCAAAGGCTATTAGCGACGCCCAGATCACCGCGAGCTCCTACTTCACTAACATGTTCGCCACCTGGAGCCCC |
| | TCCAAGGCCAGGCTGCACTTGCAGGGACGGTCAAATGCCTGGCGGCCGCAAGTGAACAATCCGAAGGAATGGCT |
| | TCAAGTGGATTTCCAAAAGACCATGAAAGTGACCGGAGTCACCACCCAGGGAGTGAAGTCCCTTCTGACCTCGA |
| | TGTATGTGAAGGAGTTCCTGATTAGCAGCAGCCAGGACGGGCACCAGTGGACCCTGTTCTTCCAAAACGGAAAG |
| | GTCAAGGTGTTCCAGGGGAACCAGGACTCGTTCACACCCGTGGTGAACTCCCTGGACCCCCCACTGCTGACGCG |
| | GTACTTGAGGATTCATCCTCAGTCCTGGGTCCATCAGATTGCATTGCGAATGGAAGTCCTGGGCTGCGAGGCCC |
| | AGGACCTGTACTGA |

1. A nucleic acid molecule comprising a first inverted terminal repeat (ITR) and a second ITR flanking a genetic cassette comprising a heterologous polynucleotide sequence, wherein:
the first ITR comprises a polynucleotide sequence at least about 75% identical to:
nucleotides 1-49, 50-58, and 59-125 of SEQ ID NO:1,
nucleotides 1-27 and 50-114 of SEQ ID NO:15, or
SEQ ID NO: 25; and
the second ITR comprises a polynucleotide sequence at least about 75% identical to:
nucleotides 1-67, 68-76, and 77-125 of SEQ ID NO:2,
nucleotides 1-65 and 88-114 of SEQ ID NO:16, or
SEQ ID NO: 26.

2. The nucleic acid molecule of claim 1, wherein the first ITR comprises a polynucleotide sequence at least about 80%, at least about 85%, at least about 90%, at least about 95%, at least about 96%, at least about 97%, at least about 98%, at least about 99%, or 100% identical to:
nucleotides 1-49, 50-58, and 59-125 of SEQ ID NO:1,
nucleotides 1-27 and 50-114 of SEQ ID NO:15, or
SEQ ID NO: 25.

3. (canceled)

4. The nucleic acid molecule of claim 1, wherein the first ITR comprises the polynucleotide sequence set forth in SEQ ID NO:1, SEQ ID NO:3, SEQ ID NO: 5, SEQ ID NO: 9, SEQ ID NO: 13, SEQ ID NO: 15, SEQ ID NO: 17, SEQ ID NO: 19, SEQ ID NO: 21, or SEQ ID NO: 25.

5-13. (canceled)

14. The nucleic acid molecule of claim 1, wherein the second ITR comprises a polynucleotide sequence at least about 80%, at least about 85%, at least about 90%, at least about 95%, at least about 96%, at least about 97%, at least about 98%, at least about 99%, or 100% identical to:
nucleotides 1-67, 68-76, and 77-125 of SEQ ID NO:2,
nucleotides 1-65 and 88-114 of SEQ ID NO:16, or
SEQ ID NO: 26.

15. (canceled)

16. The nucleic acid molecule of claim 1, wherein the second ITR comprises the polynucleotide sequence set forth in SEQ ID NO:2, SEQ ID NO: 4, SEQ ID NO: 6, SEQ ID NO: 10, SEQ ID NO: 14, SEQ ID NO: 16, SEQ ID NO: 18, SEQ ID NO: 20, SEQ ID NO: 22, SEQ ID NO: 26.

17-25. (canceled)

26. A nucleic acid molecule comprising a first inverted terminal repeat (ITR) and a second ITR flanking a genetic cassette comprising a heterologous polynucleotide sequence,
wherein the first ITR comprises SEQ ID NOs:1, 3, 5, 9, 13, 15, 17, 19, 21, 23, or 25 and the second ITR comprises SEQ ID NOs:2, 4, 6, 10, 14, 16, 18, 20, 22, 24, or 26.

27. The nucleic acid molecule of claim 26, wherein the genetic cassette further comprises one or more of: a promoter, an enhancer, an intronic sequence, a post-transcriptional regulatory element, and/or a 3′UTR poly(A) tail sequence.

28-43. (canceled)

44. The nucleic acid molecule of claim 26, wherein the nucleic acid molecule comprises from 5′ to 3′: the first ITR, the genetic cassette, and the second ITR, wherein the genetic cassette comprises a tissue-specific promoter sequence, an intronic sequence, the heterologous polynucleotide sequence, a post-transcriptional regulatory element, and a 3′UTR poly(A) tail sequence.

45. (canceled)

46. The nucleic acid molecule of claim 26, wherein the genetic cassette is a single stranded nucleic acid or is a double stranded nucleic acid.

47-56. (canceled)

57. The nucleic acid molecule of claim 26, wherein the heterologous polynucleotide sequence encodes a clotting factor, wherein the clotting factor comprises factor I (FI), factor II (FII), factor III (FIII), factor IV (FIV), factor V (FV), factor VI (FVI), factor VII (FVII), factor VIII (FVIII), factor IX (FIX), factor X (FX), factor XI (FXI), factor XII (FXII), factor XIII (FXIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI-2), or any combination thereof.

58-66. (canceled)

67. A vector comprising the nucleic acid molecule of claim 26.

68. A host cell comprising the nucleic acid molecule of claim 26.

69. A pharmaceutical composition comprising the nucleic acid of claim 26.

70-72. (canceled)

73. A baculovirus system for production of the nucleic acid molecule of claim 26.

74-75. (canceled)

76. A method of expressing a heterologous polynucleotide sequence in a subject in need thereof, comprising administering to the subject the nucleic acid molecule of claim 26.

77. A method of treating a disease or disorder in a subject in need thereof, comprising administering to the subject the nucleic acid molecule of claim 26.

78-83. (canceled)

84. The nucleic acid molecule of claim 44, wherein the genetic cassette comprises the nucleotide sequence of SEQ ID NO: 27.

85-87. (canceled)


SEQUENCE LISTING (37 sequences)

SEQ ID NO: 1 - DNA, 125 bp, Artificial Sequence
  Synthetic polynucleotide sequence.
ccaaatcagatgccgccggtcgccgccggtaggcgggacttccggtacagcgcgccgctgtaccggaagtcccgcctaccggcggcgaccggcggcatctgatttggtgtcttcttttaaatttt

SEQ ID NO: 2 - DNA, 125 bp, Artificial Sequence
  Synthetic polynucleotide sequence.
aaaatttaaaagaagacaccaaatcagatgccgccggtcgccgccggtaggcgggacttccggtacagcggcgcgctgtaccggaagtcccgcctaccggcggcgaccggcggcatctgatttgg

SEQ ID NO: 3 - DNA, 160 bp, Artificial Sequence
  Synthetic polynucleotide sequence.
ccaaatcagatgccgccggtcgccgccggtaggcgggacttccggtacaagatggcggacaattagcggcgcgccgctaattgtccgccatcttgtaccggaagtcccgcctaccggcggcgaccggcggcatctgatttggtgtcttcttttaaatttt

SEQ ID NO: 4 - DNA, 160 bp, Artificial Sequence
  Synthetic polynucleotide sequence.
aaaatttaaaagaagacaccaaatcagatgccgccggtcgccgccggtaggcgggacttccggtacaagatggcggacaattagcggcgcgccgctaattgtccgccatcttgtaccggaagtcccgcctaccggcggcgaccggcggcatctgatttgg

SEQ ID NO: 5 - DNA, 232 bp, Artificial Sequence
  Synthetic polynucleotide sequence.
ccaaatcagatgccgccggtcgccgccggtaggcgggacttccggtacaagatggcggacaattacgtcatttcctgtgacgtcatttcctgtgacgtcacgcggcgcgccgcgtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 6 - DNA, 232 bp, Artificial Sequence
  Synthetic polynucleotide sequence.
aaaatttaaaagaagacaccaaatcagatgccgccggtcgccgccggtaggcgggacttccggtacaagatggcggacaattacgtcatttcctgtgacgtcatttcctgtgacgtcacgcggcgcgccgcgtgacgtcacaggaaatgacgtcacaggaaatgacgtaattgtccgccatcttgtaccggaagtcccgcctaccggcggcgaccggcggcatctgatttgg

SEQ ID NO: 7 - DNA, 248 bp, Artificial Sequence
  Synthetic polynucleotide sequence.
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 8 - DNA, 248 bp, Artificial Sequence
  Synthetic polynucleotide sequence.
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagag

SEQ ID NO: 9 - DNA, 252 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttcgcatgcgaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 10 - DNA, 252 bp, Artificial Sequence
  Synthetic polynucleotide sequence.
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttcgcatgcgaactacgtcaccggatgtgcgtcaccggaagcatgtgaccggaacttgcgtcacttccccctcccctgattggctggttcgaacgaacgaaccctccaatgag

SEQ ID NO: 11 - DNA, 282 bp, Artificial Sequence
  Synthetic polynucleotide sequence.
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 12 - DNA, 282 bp, Artificial Sequence
  Synthetic polynucleotide sequence.
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccg

SEQ ID NO: 13 - DNA, 324 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcgcatgctcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 14 - DNA, 324 bp, Artificial Sequence
  Synthetic polynucleotide sequence.
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgagcatgcgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgag

SEQ ID NO: 15 - DNA, 114 bp, Artificial Sequence
  Synthetic polynucleotide sequence.
ctcattggagggttcgttcgttcgaacgttcgttcgcatgcgaacgaacgttcgaacgaacgaaccctccaatgagactcaaggacaagaggatattttgcgcgccaggaagtg

SEQ ID NO: 16 - DNA, 114 bp, Artificial Sequence
  Synthetic polynucleotide sequence.
cacttcctggcgcgcaaaatatcctcttgtccttgagtctcattggagggttcgttcgttcgaacgttcgttcgcatgcgaacgaacgttcgaacgaacgaaccctccaatgag

SEQ ID NO: 17 - DNA, 347 bp, Artificial Sequence
  Synthetic polynucleotide sequence.
ccaaatcagatgccgccggtcgccgccggtaggcgggacttccggtacaagatggcggacaattacgtcatttcctgtgacgtcatttcctgtgacgtcacgcggcgcgcctattgcttggcagcgcctatcgccaggtattactccaatcccgaatatccgagatcgggatcacccgagagaagttcaacctacatcctcaatcccgatctatccgaggcgcgccgcgtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 18 - DNA, 341 bp, Artificial Sequence
  Synthetic polynucleotide sequence.
aaaatttaaaagaagacaccaaatcagatgccgccggtcgccgccggtaggcgggacttccggtacaagatggcggacaattacgtcatttcctgtgacgtcatttcctgtgacgtcacgcggcgcgccagcgcctatcgccaggtattactccaatcccgaatatccgagatcgggatcacccgagagaagttcaacctacatcctcaatcccgatctatccgagatccggcgcgccgcgtgacgtcacaggaaatgacgtcacaggaaatgacgtaattgtccgccatcttgtaccggaagtcccgcctaccggcggcgaccggcggcatctgatttgg

SEQ ID NO: 19 - DNA, 408 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgcgaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 20 - DNA, 408 bp, Artificial Sequence
  Synthetic polynucleotide sequence.
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgcgaactacgtcaccggatgtgcgtcaccggaagcatgtgaccggaacttgcgtcacttccccctcccctgattggctggttcgaacgaacgaaccctccaatgag

SEQ ID NO: 21 - DNA, 480 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcgcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacgcatgctcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 22 - DNA, 481 bp, Artificial Sequence
  Synthetic polynucleotide sequence.
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgagcatgcctgtctatcgcctacccatccctgtctgagatcaagggcgtgatcgtgcacagactggagagcgtgtcctataatatcggctctcaggagtggagcaccacagtgcccagatacgtggccacccagggctatctgatctccaacttcgacggcatgcgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgag

SEQ ID NO: 23 - DNA, 383 bp, Artificial Sequence
  B9 WT 5′
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 24 - DNA, 383 bp, Artificial Sequence
  B19 WT 3′
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 25 - DNA, 153 bp, Artificial Sequence
  HBoV1 WT ITR 5′
ttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgc

SEQ ID NO: 26 - DNA, 150 bp, Artificial Sequence
  HBoV1 WT ITR 3′
tatatgtgacgtggttgtacagacgccatcttggaatccaatatgtctgccggcgattagatcatgcgcgcgcgcagcgcgctgcgcgcagcgcaggcatgactgagccggcagacatattggattccaagatggcgtctgtacaaccac

SEQ ID NO: 27 - DNA, 7891 bp, Artificial Sequence
  V2.0 Human codon optimized mTTR-Intron-BDD- FVIIIXTEN-WPRE-bGHPoly expression cassette
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaag

SEQ ID NO: 28 - DNA, 4824 bp, Artificial Sequence
  Nucleotide sequence encoding BDDcoFVIII fused with 144 amino acid XTEN
atgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 29 - DNA, 8257 bp, Artificial Sequence
  Human codon optimized HBoV1-5′ITR-mTTR-Intron-BDD-FVIIIXTEN-WPRE-bGHPoly-HBoV1-3′ITR expression cassette
gtatacctgcaggctagccacgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcaagcttggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaagtatatgtgacgtggttgtacagacgccatcttggaatccaatatgtctgccggcgattagatcatgcgcgcgcgcagcgcgctgcgcgcagcgcaggcatgactgagccggcagacatattggattccaagatggcgtctgtacaaccacgtgcttaagctgcagactagtgagctcgttaac

SEQ ID NO: 30 - DNA, 330 bp, Artificial Sequence
  A1MB2 ENHANCER
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccacc

SEQ ID NO: 31 - DNA, 345 bp, Artificial Sequence
  mTTR promoter
gatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctag

SEQ ID NO: 32 - DNA, 1489 bp, Artificial Sequence
  CHIMERIC INTRON
tcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaatta

SEQ ID NO: 33 - DNA, 595 bp, Artificial Sequence
  WPRE
tcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctg

SEQ ID NO: 34 - DNA, 211 bp, Artificial Sequence
  bGHpA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 35 - DNA, 4317 bp, Artificial Sequence
  Nucleotide sequence of coFVIII-6
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 36 - DNA, 4335 bp, Artificial Sequence
  Nucleotide sequence encoding BDDcoFVIII_noXTEN
gccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccggcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 37 - DNA, 4824 bp, Artificial Sequence
  V1.0 Human codon optimized TTP-Intron-BDD-FVIIIco6XTEN-WPRE-bGHPoly expression cassette
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga